(19) United States
(12) Patent Application Publication
(10) Pub. No.: US 2024/0353322 A1
(43) Pub. Date: Oct. 24, 2024

(54) COMBINED DETERMINATION OF THE CONCENTRATION AND ENANTIOMERIC COMPOSITION OF CHIRAL COMPOUNDS USING SINGLE CHIROPTICAL ASSAY

(71) Applicant: GEORGETOWN UNIVERSITY, Washington, DC (US)

(72) Inventors: Christian WOLF, Arlington, VA (US); Archita SRIPADA, Arlington, VA (US); Fathima Yushra THANZEEL, Jersey City, NJ (US)

(21) Appl. No.: 18/682,596

(22) PCT Filed: Jun. 10, 2022
(86) PCT No.: PCT/US2022/032977

§ 371 Date: Feb. 9, 2024

Related U.S. Application Data
(60) Provisional application No. 63/231,835, filed on Aug. 11, 2021.

(51) Int. Cl. G01N 21/31; C09B 57/00

(52) U.S. Cl. CPC G01N 21/31; C09B 57/00

(57) ABSTRACT

The present invention relates to the analytical methods for determining the concentration of the analyte in the sample and one or both of (i) the absolute configuration of the analyte in the sample, and (ii) the enantiomeric and/or the diastereomeric composition of the analyte in the sample and the kit for carrying out the analytical method. The present invention also relates to the use of a chromophore probe in such analytical methods.

This application claims the priority benefit of U.S. Provisional Patent Application Ser. No. 63/231,835, filed Aug. 11, 2021, which is hereby incorporated by reference in its entirety.

This invention was made with government support under grant number CHE-1764135 awarded by the National Science Foundation. The government has certain rights in the invention.

### FIELD OF THE INVENTION

The present application relates to an analytical method using a single chiroptical assay format for the determination of the concentration of a chiral analyte in a sample and one or both of (i) the absolute configuration of the analyte in the sample, and (ii) the enantiomeric and/or the diastereomeric composition of the analyte in the sample.

### BACKGROUND OF THE INVENTION

Chirality plays an essential role in nature and throughout the chemical sciences. Enantioselective synthesis and analysis of chiral compounds have become central aspects of drug discovery, material sciences, and other rapidly expanding research areas. The importance of chiral compounds in the pharmaceutical industry and other fields has stimulated the development of numerous asymmetric catalysts and reaction strategies (Gawley & Aubé, “Principles of Asymmetric Synthesis,” in Tetrahedron Organic Chemistry Series, J. E. Baldwin & P. D. Magnus eds., Elsevier Press (1996); Wolf C., “Dynamic Stereochemistry of Chiral Compounds,” The Royal Society of Chemistry 180-398 (2008)). Optimization efforts typically entail elaborate chiral ligand modifications to fine-tune the catalyst in addition to conventional screening of a wide range of reaction parameters.

The advance of combinatorial methods and automated synthesis allows the production of large numbers of chiral samples literally overnight. The steadily increasing efficiency in asymmetric synthesis has shifted focus toward the development of time efficient optical techniques with potential for high-throughput screening (Leung et al., Chem. Soc. Rev. 41:448 (2012)). In contrast to the advance of asymmetric synthesis, the analysis of the enantiomeric composition of chiral products is typically time-consuming and delays the discovery progress (Leung et al., Chem. Soc. Rev. 41:448-79 (2012)). Several groups have begun to address this bottleneck with the development of optical methods based on fluorescence (Lee & Lin, J. Am. Chem. Soc. 124:4554-55 (2002); Lin et al., J. Am. Chem. Soc. 124:2088-89 (2002); Mei & Wolf, Chem. Commun. 2078-79 (2004); Zhao et al., Angew. Chem. Int. Ed. 43:3461-64 (2004); Mei & Wolf, J. Am. Chem. Soc. 126:14736-37 (2004); Li et al., Angew. Chem. Int. Ed. 44:1690-93 (2005); Tumambac & Wolf, Org. Lett. 7:4045-48 (2005); Mei et al., J. Org. Chem. 71:2854-61 (2006); Mei & Wolf, Tetrahedron Lett. 47:7901-04 (2006); Wolf et al., Chem. Commun. 40:4242-44 (2006); Liu et al., J. Org. Chem. 73:4267-70 (2008); Yu & Pu, J. Am. Chem. Soc. 132:17698-700 (2010); Wu et al., Chem. Eur. J. 17:7632-44 (2011); Yang et al., Org. Lett. 13:3510-13 (2011); He et al., Chem. Commun. 47:11641-43 (2011); Wanderley et al., J. Am. Chem. Soc. 134:9050-53 (2012); Pu, Review, Chem. Rev. 104:1687-716 (2004)), UV absorbance (Zhu & Anslyn, J. Am. Chem. Soc. 126:3676-77 (2004); Mei & Wolf, J. Am. Chem. Soc. 128:13326-27 (2006); Leung et al., J. Am. Chem. Soc. 130:12318-27 (2008); Leung & Anslyn, J. Am. Chem. Soc. 130:12328-33 (2008); Iwaniuk et al., J. Org. Chem. 77:5203-08 (2012)), and circular dichroism (Superchi et al., Angew. Chem. Int. Ed 40:451-54 (2001); Kurtan et al., J. Am. Chem. Soc. 123:5974-82 (2001); Huang et al., J. Am. Chem. Soc. 124:10320-35 (2002); Mazaleyrat et al., J. Am. Chem. Soc. 126:12874-79 (2004); Superchi et al., J. Am. Chem. Soc. 128:6893-902 (2006); Holmes et al., J. Am. Chem. Soc. 129:1506-07 (2007); Dutot et al., J. Am. Chem. Soc. 130:5986-92 (2008); Kim et al., Angew. Chem. Int. Ed. 47:8657-60 (2008); Waki et al., Angew. Chem. Int. Ed. 46:3059-61 (2007); Katoono et al., J. Am. Chem. Soc. 131:16896-904 (2009); Ghosn & Wolf, J. Am. Chem. Soc. 131:16360-61 (2009); Ghosn & Wolf, Tetrahedron 66:3989-94 (2010); Ghosn & Wolf, J. Org. Chem. 76:3888-97 (2011); Ghosn & 20 Wolf, Tetrahedron 67:6799-803 (2011); Joyce et al., J. Am. Chem. Soc. 133:13746-52 (2011); You et al., J. Am. Chem. Soc. 134:7117-25 (2012); Wezenberg et al., Angew. Chem. Int. Ed. 50:713-16 (2011); Iwaniuk & Wolf, J. Am. Chem. Soc. 133:2414-17 (2011); Iwaniuk & Wolf, Org. Lett. 13:2602-05 (2011); Iwaniuk et al., Chirality 24:584-89 (2012); Li et al., J. Am. Chem. Soc. 134:9026-29 (2012); Iwaniuk & Wolf, Chem. Commun. 48:11226-28 (2012)).

Circular dichroism (CD) spectroscopy is one of the most powerful techniques commonly used for elucidation of the three-dimensional structure, molecular recognition events, and stereodynamic processes of chiral compounds (Gawroniski & Grajewski, Org. Lett. 5:3301-03 (2003); Allenmark, Chirality 15:409-22 (2003); Berova et al., Chem. Soc. Rev. 36:914-31 (2007)). The potential of chiroptical CD and circular polarized luminescence (CPL) assays with carefully designed probes that produce a circular dichroism signal upon recognition of a chiral substrate has received increasing attention in recent years, and bears considerable promise with regard to high-throughput enantiomeric excess (ee) screening (Nieto et al., J. Am. Chem. 130:9232-33 (2008); Leung et al., Chem. Soc. Rev. 41:448-79 (2012); Song et al., Chem. Commun. 49:5772-74 (2013)).

Many examples of chirality chemosensing with stereodynamic molecular receptors or supramolecular arrangements that generate a characteristic CD signal upon covalent or non-covalent binding of a target compound have been reported (e.g., Bentley & Wolf, J. Am. Chem. Soc. 135:12200 (2013); Ghosn & Wolf, J. Am. Chem. Soc. 131:16360 (2009); Ghosn & Wolf, J. Org. Chem. 76:3888 (2011); Ghosn & Wolf, Tetrahedron 67:6799 (2011); Hembury et al., Review, Chem. Rev. 108:1-73 (2008) (supramolecular sensors); Holmes et al., Chirality 14:471 (2002); Iwaniuk & Wolf, Chem. Commun. 48:11226 (2012); Iwaniuk & Wolf, J. Am. Chem. Soc. 133:2414 (2011); Iwaniuk & Wolf, Org. Lett. 13:2602 (2011); Iwaniuk et al., Chirality 24:584 (2012); Katoono et al., Tetrahedron Lett. 47:1513-18 (2006); Kawai et al., Chem. Eur. J. 11:815-24 (2005); Kohmoto et al., Tetrahedron Lett. 49:1223-27 (2008); Leung & Anslyn, Org. Lett. 13:2298 (2011); Matile et al., J. Am. Chem. Soc. 33:2072 (1993); Nieto et al., J. Am. Chem. Soc. 130:9232 (2008); Tartaglia et al., J. Org. Chem. 73:4865 (2008); Tartaglia et al., Org. Lett. 10:3421-24 (2008); Tumambac et al., Eur. J. Org. Chem. 3850-56 (2004); Wolf & Bentley, Review, Chem. Soc. Rev. 42:5408 (2013); Zhang & Wolf, Chem. Comm. 49:7010 (2013)). This includes biphenyl-derived probes that populate a thermodynamically favored chiral conformation upon reaction with one enantiomer of an amino acid, carboxylic acid, amine, or alcohol. This chiral induction process yields a Cotton effect that can be correlated to the absolute configuration of the covalently-bound substrate (Superchi et al., Angew. Chem. Int. Ed. 40:451-54 (2001); Hosoi et al., Tetrahedron Lett. 42:6315-17 (2001); Mazaleyrat et al., J. Am. Chem. Soc. 126:12874-79 (2004); Mazaleyrat et al., Chem. Eur. J. 11:6921-29 (2005); Superchi et al., J. Am. Chem. Soc. 128:6893-902 (2006); Dutot et al., J. Am. Chem. Soc. 130:5986-92 (2008); Kuwahara et al., Org. Lett. 15:5738-41 (2013)). Essentially the same concept has been exploited for chirality chemosensing by using molecular bevel gears (Sciebura et al., Angew. Chem. Int. Ed. 48:7069-72 (2009); Sciebura & Gawronski, Chem. Eur. J. 17:13138-41 (2011)), propellers (Katoono et al., J. Am. Chem. Soc. 131:16896-904 (2009)), or other probes that can afford a CD-active helical arrangement (Waki et al., Angew. Chem. Int. Ed. 46:3059-61 (2007); Tartaglia et al., Org. Lett. 10:3421-24 (2008); Kim et al., Angew. Chem. Int. Ed. 47:8657-60 (2008)). Similarly, a variety of intriguing stereodynamic chemosensors that generate strong CD signals in the presence of a chiral bias have been developed (Balaz et al., Angew. Chem. Int. Ed 44:4006-09 (2005); Berova et al., Chem. Commun. 5958-80 (2009); Borovkov et al., J. Am. Chem. Soc. 123:2979-89 (2001); Canary et al., Chem. Commun. 46:5850-60 (2010); Holmes et al., J. Am. Chem. Soc. 129:1506-07 (2007); Huang et al., J. Am. Chem. Soc. 124:10320-35 (2002); Ishii et al., Chirality 17:305-15 (2005); Joyce et al., Chem. Eur. J. 18:8064-69 (2012); Joyce et al., J. Am. Chem. Soc. 133:13746-52 (2011); Katoono et al., Tetrahedron Lett. 47:1513-18 (2006); Kikuchi et al., J. Am. Chem. Soc. 114:1351-58 (1992); Kim et al., Chem. Commun. 49:11412-14 (2013); Kurtan et al., J. Am. Chem. Soc. 123:5962-73 (2001); Kurtan et al., J. Am. Chem. Soc. 123:5974-82 (2001); Li et al., J. Am. Chem. Soc. 130:1885-93 (2008); Li & Borhan, J. Am. Chem. Soc. 130:16126-27 (2008); Li et al., J. Am. Chem. Soc. 134:9026-29 (2012); Nieto et al., Chem. Eur. J. 16:227-32 (2010); Proni et al., Chem. Commun. 1590-91 (2002); Proni et al., J. Am. Chem. Soc. 125:12914-27 (2003); Tamiaki et al., Tetrahedron 59:10477-83 (2003); Tsukube et al., J. Chem. Soc. Dalton Trans. 1:11-12 (1999); Waki et al., Angew. Chem. Int. Ed. 46:3059-61 (2007); Wezenberg et al., Angew. Chem. Int. Ed. 50:713-16 (2011); Yang et al., Org. Lett. 4:3423-26 (2002); You et al., J. Am. Chem. Soc. 134:7117-25 (2012); You et al., J. Am. Chem. Soc. 134:7126-34 (2012); You et al., Nat. Chem. 3:943-48 (2011); Zhang et al., Chirality 15:180-89 (2003)). In many cases, the CD output of the chemosensor allows determination of the absolute configuration and the enantiomeric composition of the chiral analyte (Wolf & Bentley, Chem. Soc. Rev. 42:5408-24 (2013)).

Despite these advances, the analysis of the concentration and the enantiomeric composition of chiral substrates by a single optical chemosensor is a difficult task, and a practical method that is applicable to many chiral compounds and avoids time consuming derivatization and purification steps is very desirable (Nieto et al., Org. Lett. 10:5167-70 (2008); Nieto et al., Chem. Eur. J. 16:227-32 (2010); Yu et al., J. Am. Chem. Soc. 134:20282-85 (2012)).

The present invention is directed to overcoming these and other deficiencies in the art.

### SUMMARY OF THE INVENTION

A first aspect of the present application relates to an analytical method that includes the steps of: providing a sample potentially containing a chiral analyte that can exist in stereoisomeric forms; contacting the sample with a chromophore probe, wherein said contacting is carried out under conditions to permit binding of the chromophore probe to the chiral analyte, if present in the sample, to form a probe-labeled analyte; and detecting the probe-labeled analyte in the sample using a single chiroptical assay format, and determining the concentration of the analyte in the sample and one or both of (i) the absolute configuration of the analyte in the sample, and (ii) the enantiomeric and/or the diastereomeric composition of the analyte in the sample.

A second aspect of the present application relates to a kit for carrying out the analytical method described herein. The kit may include an aqueous or non-aqueous solution comprising a chromophore probe and, optionally, one or more of (i) sample tubes suitable for use with a spectrophotometer; (ii) an optically pure reference sample of an analyte, (iii) directions for using a spectrophotometer for carrying out circular dichroism (CD), vibrational CD (VCD), electronic CD, optical rotatory dispersion (ORD), or polarimetry analyses to measure the concentration of an analyte in a sample and one or both of the absolute configuration of the analyte in the sample, and the enantiomeric and/or the diastereomeric composition of the analyte in the sample, and (iv) a recordable medium comprising a template for analyzing data obtained from the spectrophotometer and determining the concentration of an analyte in a sample and one or both of the absolute configuration of the analyte in the sample, and the enantiomeric and/or the diastereomeric composition of the analyte in the sample.

A third aspect of the present application relates to the use of a chromophore probe as defined below in an analytical method of measuring the concentration of an analyte in a sample and one or both of the absolute configuration of the analyte in the sample, and the enantiomeric and/or the diastereomeric composition of the analyte in the sample.

Using achiral arylsulfonyl chloride probes in a proof of concept, the accompanying Examples demonstrate quantitative chirality sensing of more than fifty amines, amino alcohols, and all standard chiral amino acids. Importantly, the sensing method employs a strategy that allows simultaneous concentration and er analysis based on the exclusive use of a single chiroptical assay format (e.g., CD measurements). Using these exemplary probes, the demonstrated chiroptical sensing is based on fast sulfonamide bond formation with stoichiometric probe amounts and can be performed in typical organic solvents or in aqueous solution by a simple mix-and-measure protocol and without the need to exclude air and moisture or other precautions, 2-Nitrobenzenesulfonyl chloride gave strong Cotton effects at long wavelengths with both aliphatic and aromatic substrates. The outstanding chiroptical properties, the general usefulness demonstrated with a very large group of chiral analytes, and the operational simplicity of chiral compound sensing with this sensor are highly advantageous features and pose an attractive alternative to traditional NMR spectroscopy or chiral chromatography methods. This conceptually new approach toward determination of both concentration and er values using a single chiroptical assay format offers significant speed, labor, and cost advantages at reduced chemical waste production compared to traditional techniques. The exemplary unified CD sensing is, of course, not limited to the use of arylsulfonyl chloride probes, but it is expected to become broadly useful with a variety of probes, and can be easily adapted by many laboratories.

### BRIEF DESCRIPTION OF THE DRAWINGS

FIG. 1 is a graph showing circular dichroism (CD) sensing of (R)-1-phenylethylamine with arylsulfonyl chlorides 1-4 (structures shown in Example 1). CD measurements were taken at 0.40 mM in ACN.

FIG. 2 is a graph showing CD sensing of (S)-3,3-diethyl-2-butylamine with arylsulfonyl chlorides 1, 2, and 4. CD measurements were taken at 0.40 mM in ACN.

FIG. 3 is a graph showing CD spectra obtained from probe 5 with (R)-1-phenylethylamine and (S)-1-phenylethylamine. A solution of probe 5 (25.0 mM), 1-phenylethylamine (20.0 mM), and Et₃N (40.0 mM) in 1.0 mL of acetonitrile was stirred for 2 hours and subjected to CD analysis. CD measurements were taken at 0.20 mM in ACN. The structure of probe 5 is shown in Example 1.

FIG. 4 is a graph showing CD spectra obtained from probe 6, 9, and 10 with (R)-1-phenylethylamine. CD measurements were taken at 0.40 mM in ACN. The structures of probe 6, 9, and 10 are shown in Example 1.

FIG. 5 shows NMR analysis of the reaction between probe 1 and 1-phenylpropan-1-amine, 15.

FIG. 6 shows NMR analysis of the reaction between probe 1 and 1-(pyridin-2-yl)ethan-1-amine, 16.

FIG. 7 shows NMR analysis of the reaction between probe 1 and 3,3-dimethylbutan-2-amine, 22.

FIG. 8 shows NMR analysis of the reaction between probe 1 and alanine.

FIG. 9 shows NMR analysis of the reaction between probe 1 and proline.

FIG. 10 shows CD analysis of the reaction between (R)-serine and probe 1. CD measurements were taken at 0.17 mM in ACN.

FIG. 11 shows ESI-MS spectrum of the reaction between (R,R)-diaminocyclohexane and probe 1 (negative ion mode).

FIG. 12 shows CD spectra obtained from probe 1 with (R)-11 and (S)-11. CD measurements were taken at 0.34 mM in ACN.

FIG. 13 shows CD spectra obtained from probe 1 with (R)-12 and (S)-12. CD measurements were taken at 0.34 mM in ACN.

FIG. 14 shows CD spectra obtained from probe 1 with (R)-13 and (S)-13. CD measurements were taken at 0.45 mM in ACN.

FIG. 15 shows CD spectra obtained from probe 1 with (R)-14 and (S)-14. CD measurements were taken at 0.91 mM in ACN.

FIG. 16 shows CD spectra obtained from probe 1 with (R)-15 and (S)-15. CD measurements were taken at 0.66 mM in ACN.

FIG. 17 shows CD spectra obtained from probe 1 with (R)-16 and (S)-16. CD measurements were taken at 0.45 mM in ACN.

FIG. 18 shows CD spectra obtained from probe 1 with (R)-17. CD measurements were taken at 0.45 mM in ACN.

FIG. 19 shows CD spectra obtained from probe 1 with (R)-18 and (S)-18. CD measurements were taken at 1.1 mM in ACN.

FIG. 20 shows CD spectra obtained from probe 1 with (R)-19. CD measurements were taken at 0.45 mM in ACN.

FIG. 21 shows CD spectra obtained from probe 1 with (R)-20 and (S)-20. CD measurements were taken at 0.91 mM in ACN.

FIG. 22 shows CD spectra obtained from probe 1 with (R)-21 and (S)-21. CD measurements were taken at 0.45 mM in ACN.

FIG. 23 shows CD spectra obtained from probe 1 with (R)-22 and (S)-22. CD measurements were taken at 0.45 mM in ACN.

FIG. 24 shows CD spectra obtained from probe 1 with (S)-23. CD measurements were taken at 0.45 mM in ACN.

FIG. 25 shows CD spectra obtained from probe 1 with (1R,2R,3R,5S)-24 (blue). CD measurements were taken at 0.45 mM in ACN.

FIG. 26 shows CD spectra obtained from probe 1 with (R)-25 and (S)-25. CD measurements were taken at 0.45 mM in ACN.

FIG. 27 shows CD spectra obtained from probe 1 with (R)-26 and (S)-26. CD measurements were taken at 0.45 mM in ACN.

FIG. 28 shows CD spectra obtained from probe 1 with (R)-27 and (S)-27. CD measurements were taken at 2.3 mM in ACN.

FIG. 29 shows CD spectra obtained from probe 1 with (R)-28 and (S)-28. CD measurements were taken at 0.45 mM in ACN.

FIG. 30 shows CD spectra obtained from probe 1 with (1R,2R)-29 and (1S,2S)-29. A solution prepared from probe 1 (100.0 mM in ACN, 480.0 μL), chiral diamine 29 (50.0 mM in ACN, 400.0 μL), and Et₃N (2.4 equivalents) was stirred for 30 minutes. CD analysis was performed after dilution with ACN as indicated below. CD measurements were taken at 0.34 mM in ACN.

FIG. 31 shows CD spectra obtained from probe 1 with (1R,2R)-30 and (1S,2S)-30. CD measurements were taken at 0.45 mM in ACN.

FIG. 32 shows CD spectra obtained from probe 1 with (1R,2S)-31 and (1S,2R)-31. CD measurements were taken at 0.45 mM in ACN.

FIG. 33 shows CD spectra obtained from probe 1 with (1R,2S)-32 and (1S,2R)-32. CD measurements were taken at 0.45 mM in ACN.

FIG. 34 shows CD spectra obtained from probe 1 with (R)-33 and (S)-33. CD measurements were taken at 0.45 mM in ACN.

FIG. 35 shows CD spectra obtained from probe 1 with (R)-34 and (S)-34. CD measurements were taken at 0.45 mM in ACN.

FIG. 36 shows CD spectra obtained from probe 1 with (R)-35 and (S)-35. CD measurements were taken at 0.45 mM in ACN.

FIG. 37 shows CD spectra obtained from probe 1 with (R)-36 and (S)-36. CD measurements were taken at 0.45 mM in ACN.

FIG. 38 shows CD spectra obtained from probe 1 with (R)-37 and (S)-37. CD measurements were taken at 0.45 mM in ACN.

FIG. 39 shows CD spectra obtained from probe 1 with (R)-38 and (S)-38. CD measurements were taken at 0.45 mM in ACN.

FIG. 40 shows CD spectra obtained from probe 1 with (R)-39 and (S)-39. CD measurements were taken at 0.45 mM in ACN.

FIG. 41 shows CD spectra obtained from probe 1 with (2R,3R)-40 and (2S,3S)-40. CD measurements were taken at 0.45 mM in ACN.

FIG. 42 shows CD spectra obtained from probe 1 with (S)-41. CD measurements were taken at 0.45 mM in ACN.

FIG. 43 shows CD spectra obtained from probe 1 with (R)-42 and (S)-42. CD measurements were taken at 0.45 mM in ACN.

FIG. 44 shows CD spectra obtained from probe 1 with (R)-43 and (S)-43. CD measurements were taken at 0.45 mM in ACN.

FIG. 45 shows CD spectra obtained from probe 1 with (R)-44 and (S)-44. CD measurements were taken at 0.45 mM in ACN.

FIG. 46 shows CD spectra obtained from probe 1 with (S)-Ala and (R)-Ala. The CD measurements were taken at 0.54 mM analyte concentration.

FIG. 47 shows CD spectra obtained from probe 1 with (S)-Val and (R)-Val. The CD measurements were taken at 0.54 mM analyte concentration.

FIG. 48 shows CD spectra obtained from probe 1 with (S)-Leu and (R)-Leu. The CD measurements were taken at 0.54 mM analyte concentration.

FIG. 49 shows CD spectra obtained from probe 1 with (S)-Ile and (R)-Ile. The CD measurements were taken at 0.54 mM analyte concentration.

FIG. 50 shows CD spectra obtained from probe 1 with (S)-Phe and (R)-Phe. The CD measurements were taken at 0.54 mM analyte concentration.

FIG. 51 shows CD spectra obtained from probe 1 with (S)-Pro and (R)-Pro. The CD measurements were taken at 0.54 mM analyte concentration.

FIG. 52 shows CD spectra obtained from probe 1 with (S)-Ser and (R)-Ser. The CD measurements were taken at 0.54 mM analyte concentration.

FIG. 53 shows CD spectra obtained from probe 1 with (S)-Thr and (R)-Thr. The CD measurements were taken at 0.54 mM analyte concentration.

FIG. 54 shows comparison of the CD intensity in the presence of 1.0 and 2.0 equivalents of probe. Solutions of probe 1 (25.0 mM in acetonitrile, 480.0 μL) and tyrosine with 4.0 equivalents of K₂CO₃ (25.0 mM in water, 400.0 μL) were combined and acetonitrile was used to dilute the total volume to 2.0 mL. The reaction mixture was stirred for 90 minutes and CD measurements were taken by diluting 120.0 μL of this mixture with 2.0 mL of acetonitrile. The above procedure was repeated with 2.0 equivalents of the probe. The CD measurements were taken at 0.27 mM analyte concentration.

FIG. 55 shows CD spectra obtained from probe 1 with (S)-Cys and (R)-Cys. The CD measurements were taken at 0.54 mM analyte concentration.

FIG. 56 shows comparison of the CD intensity using 1.0 and 2.0 equivalents of probe 1 for Cys sensing. The CD measurements were taken at 0.27 mM analyte concentration.

FIG. 57 shows CD spectra obtained from probe 1 with (S)-Met and (R)-Met. The CD measurements were taken at 0.54 mM analyte concentration.

FIG. 58 shows CD spectra obtained from probe 1 with (S)-Asn and (R)-Asn. The CD measurements were taken at 0.54 mM analyte concentration.

FIG. 59 shows CD spectra obtained from probe 1 with (S)-Gln and (R)-Gln. The CD measurements were taken at 0.54 mM analyte concentration.

FIG. 60 shows CD spectra obtained from probe 1 with (S)-Trp and (R)-Trp. The CD measurements were taken at 0.54 mM analyte concentration.

FIG. 61 shows CD spectra obtained from probe 1 with (S)-Asp and (R)-Asp. The CD measurements were taken at 0.54 mM analyte concentration.

FIG. 62 shows CD spectra obtained from probe 1 with (S)-Glu and (R)-Glu. The CD measurements were taken at 0.54 mM analyte concentration.

FIG. 63 shows comparison of the CD intensity in the presence of 1.0 and 2.0 equivalents of probe 1. Solutions of probe 1 (25.0 mM in acetonitrile, 480.0 μL) and lysine monohydrochloride together with 4 equivalents of K₂CO₃ (25.0 mM in water, 400.0 μL) were combined and acetonitrile was added to dilute the total volume to 2.0 mL. The reaction mixture was stirred for 90 minutes and CD measurements were taken by diluting 120.0 μL of this mixture with 2.0 mL of acetonitrile. The above procedure was repeated with 2.0 equivalents of the probe. The CD measurements were taken at 0.27 mM analyte concentration.

FIG. 64 shows CD spectra obtained from probe 1 with (S)-Lys and (R)-Lys with 1.0 equivalent of the probe. The CD measurements were taken at 0.54 mM analyte concentration.

FIG. 65 shows CD spectra obtained from probe 1 with (S)-Arg and (R)-Arg. The CD measurements were taken at 0.54 mM analyte concentration.

FIG. 66 shows CD spectra obtained from probe 1 with (S)-His and (R)-His. The CD measurements were taken at 0.54 mM analyte concentration.

FIG. 67 shows chiroptical response of probe 1 to scalemic mixtures of 3,3-dimethylbutan-2-amine.

FIG. 68 shows plot of the CD amplitudes at 325.5 nm and 254.0 nm versus sample ee for 3,3-dimethylbutan-2-amine.

FIG. 69 shows plot of CD signals obtained by addition of varying amounts of the sensor 1 to a nonracemic (R)-3,3-dimethylbutan-2-amine sample (12.0 mM, 92.0:8.0 er).

FIG. 70 shows plot of CD amplitudes at 324.0 nm obtained with varying amounts of the sensor and a nonracemic (R)-3,3-dimethylbutan-2-amine sample (12.0 mM, 92.0:8.0 er).

FIG. 71 shows plot of CD signals obtained by addition of varying amounts of the sensor 1 to a nonracemic (R)-3,3-dimethylbutan-2-amine sample (14.0 mM, 65.0:35.0 er).

FIG. 72 shows plot of CD amplitudes at 324.0 nm obtained with varying amounts of the sensor and a nonracemic (R)-3,3-dimethylbutan-2-amine sample (14.0 mM, 65.0:35.0 er).

FIG. 73 shows plot of CD signals obtained by addition of varying amounts of the sensor 1 to a nonracemic (R)-3,3-dimethylbutan-2-amine sample (14.0 mM, 96.0:4.0 er).

FIG. 74 shows plot of CD amplitudes at 324.0 nm obtained with varying amounts of the sensor and a nonracemic (R)-3,3-dimethylbutan-2-amine sample (14.0 mM, 96.0:4.0 er).

FIG. 75 shows plot of CD signals obtained by addition of varying amounts of the sensor 1 to a nonracemic (R)-3,3-dimethylbutan-2-amine sample (8.0 mM, 81.5:18.5 er).

FIG. 76 shows plot of CD amplitudes at 324.0 nm obtained with varying amounts of the sensor and a nonracemic (R)-3,3-dimethylbutan-2-amine sample (8.0 mM, 81.5:18.5er).

FIG. 77 shows plot of CD signals obtained by addition of varying amounts of the sensor 1 to a nonracemic (R)-3,3-dimethylbutan-2-amine sample (6.0 mM, 55.0:45.0 er).

FIG. 78 shows plot of CD amplitudes at 324.0 nm obtained with varying amounts of the sensor and a nonracemic (R)-3,3-dimethylbutan-2-amine sample (6.0 mM, 55.0:45.0 er).

FIG. 79 shows plot of CD signals obtained by addition of varying amounts of the sensor 1 to a nonracemic (S)-3,3-dimethylbutan-2-amine (5.0 mM, 55.0:45.0 er).

FIG. 80 shows plot of CD amplitudes at 324.0 nm obtained with varying amounts of the sensor and a nonracemic (S)-3,3-dimethylbutan-2-amine sample (5.0 mM, 55.0:45.0 er).

FIG. 81 shows plot of CD signals obtained by addition of varying amounts of the sensor 1 to a nonracemic (S)-3,3-dimethylbutan-2-amine sample (7.0 mM, 68.5:31.5 er).

FIG. 82 shows plot of CD amplitudes at 324.0 nm obtained with varying amounts of the sensor and a nonracemic (S)-3,3-dimethylbutan-2-amine sample (7.0 mM, 68.5:31.5er).

FIG. 83 shows plot of CD signals obtained by addition of varying amounts of the sensor 1 to a nonracemic (S)-3,3-dimethylbutan-2-amine sample (10.0 mM, 85.0:15.0 er).

FIG. 84 shows plot of CD amplitudes at 324.0 nm obtained with varying amounts of the sensor and a nonracemic (S)-3,3-dimethylbutan-2-amine sample (10.0 mM, 85.0:15.0 er).

FIG. 85 shows plot of CD signals obtained by addition of varying amounts of the sensor 1 to a nonracemic (S)-3,3-dimethylbutan-2-amine sample (13.0 mM, 52.5:47.5 er).

FIG. 86 shows plot of CD amplitudes at 324.0 nm obtained with varying amounts of the sensor and a nonracemic (S)-3,3-dimethylbutan-2-amine sample (13.0 mM, 52.5:47.5 er).

FIG. 87 shows plot of CD signals obtained by addition of varying amounts of the sensor 1 to a nonracemic (S)-3,3-dimethylbutan-2-amine sample (9.0 mM, 99.0:1.0 er).

FIG. 88 shows plot of CD amplitudes at 324.0 nm obtained with varying amounts of the sensor and a nonracemic (S)-3,3-dimethylbutan-2-amine sample (9.0 mM, 99.0:1.0 er).

FIG. 89 shows plot of CD signals obtained by addition of varying amounts of the sensor 1 to a nonracemic L-alanine sample (20.0 mM, 55:45 er).

FIG. 90 shows plot of CD amplitudes at 327.0 nm obtained with varying amounts of the sensor and a nonracemic L-alanine sample (20.0 mM, 55:45 er).

FIG. 91 shows plot of CD signals obtained by addition of varying amounts of the sensor 1 to a nonracemic D-alanine sample (19.0 mM, 97.5:2.5 er).

FIG. 92 shows plot of CD amplitudes at 327.0 nm obtained with varying amounts of the sensor and a nonracemic D-alanine sample (19.0 mM, 97.5:2.5 er).

FIG. 93 shows plot of CD signals obtained by addition of varying amounts of the sensor 1 to a nonracemic D-alanine sample (17.0 mM, 88.0:12.0 er).

FIG. 94 shows plot of CD amplitudes at 327.0 nm obtained with varying amounts of the sensor and a nonracemic D-alanine sample (17.0 mM, 88.0:12.0 er).

FIG. 95 shows plot of CD signals obtained by addition of varying amounts of the sensor 1 to a nonracemic D-alanine sample (15.0 mM, 76.0:24.0 er).

FIG. 96 shows plot of CD amplitudes at 327.0 nm obtained with varying amounts of the sensor and a nonracemic D-alanine sample (15.0 mM, 76.0:24.0 er).

FIG. 97 shows plot of CD signals obtained by addition of varying amounts of the sensor 1 to a nonracemic D-alanine sample (13.0 mM, 66.0:34.0 er).

FIG. 98 shows plot of CD amplitudes at 327.0 nm obtained with varying amounts of the sensor and a nonracemic D-alanine sample (13.0 mM, 66.0:34.0 er).

FIG. 99 shows plot of CD signals obtained by addition of varying amounts of the sensor 1 to a nonracemic D-alanine sample (11.0 mM, 55.0:45.0 er).

FIG. 100 shows plot of CD amplitudes at 327.0 nm obtained with varying amounts of the sensor and a nonracemic D-alanine sample (11.0 mM, 55.0:45.0 er).

FIG. 101 shows plot of CD signals obtained by addition of varying amounts of the sensor 1 to a nonracemic L-alanine sample (14.0 mM, 80.5:19.5 er).

FIG. 102 shows plot of CD amplitudes at 327.0 nm obtained with varying amounts of the sensor and a nonracemic L-alanine sample (14.0 mM, 80.5:19.5 er).

FIG. 103 shows plot of CD signals obtained by addition of varying amounts of the sensor 1 to a nonracemic L-alanine sample (10.0 mM, 91.0:9.0 er).

FIG. 104 shows plot of CD amplitudes at 327.0 nm obtained with varying amounts of the sensor and a nonracemic L-alanine sample (10.0 mM, 91.0:9.0 er).

FIG. 105 shows plot of CD signals obtained by addition of varying amounts of the sensor 1 to a nonracemic L-alanine sample (12.0 mM, 85.0:15.0 er).

FIG. 106 shows plot of CD amplitudes at 327.0 nm obtained with varying amounts of the sensor and a nonracemic L-alanine sample (12.0 mM, 85.0:15.0 er).

FIG. 107 shows plot of CD signals obtained by addition of varying amounts of the sensor 1 to a nonracemic L-alanine sample (18.0 mM, 70.0:30.0 er).

FIG. 108 shows plot of CD amplitudes at 327.0 nm obtained with varying amounts of the sensor and a nonracemic L-alanine sample (18.0 mM, 70.0:30.0 er).

FIG. 109 shows plot of CD signals obtained by addition of varying amounts of the sensor 1 to a 33.0 mM KR mixture, which was diluted for the CD analysis.

FIG. 110 shows plot of CD amplitudes at 324.0 nm obtained with varying amounts of the sensor and a 33.0 mM KR mixture, which was diluted for the CD analysis. The concentration determination of 9.41 mM corresponds to 31.4 mM of the product in the original KR mixture.

FIG. 111 shows NMR reaction analysis.

FIG. 112 shows chiral HPLC separation of the enantiomers of N-(1-phenylethyl)benzamide.

FIG. 113 shows chiral HPLC reaction analysis of the crude reaction mixture derivatized with benzoyl chloride.

FIG. 114 shows crystallographic analysis of (S)—N-(3,3-dimethylbutan-2-yl)-2-nitrobenzenesulfonamide.

FIGS. 115A-B show chirality sensing concept and probe structures (FIG. 115A) and also initial CD screening results and general chiroptical sensing features (FIG. 115B). CD measurements were taken in ACN (1, 2, and 5), THE (3), and CHCl₃ (4) at 0.40 mM. The structures of probes 1-5 are shown in Example 1.

FIGS. 116A-J show structures of amines (FIG. 116A) and amino alcohols (FIG. 116F) tested (only one enantiomer is shown) and selected CD responses of the 2-nitrobenzenesulfonyl tag (FIGS. 116B-E and 116G-J). CD measurements were taken in ACN at 0.34-1.1 mM concentrations. Representative chirality sensing examples of amines are shown in FIGS. 116B-E: primary α-arylamine (FIG. 116B), α-amino amide (FIG. 116C), aliphatic amine (FIG. 116D), and secondary amine (FIG. 116E). Representative sensing examples of amino alcohols are shown in FIGS. 2G-J: α,β-diarylamino alcohol (FIG. 116G), aliphatic amino alcohol (FIG. 116H), secondary α-aryl amino alcohol (FIG. 116I), and aliphatic secondary amino alcohol (FIG. 116J).

FIGS. 117A-B show sulfonylation of 22 (FIG. 117A) and X-ray structure of 45 (FIG. 117B). FIG. 117C shows CD responses for (R)-22 and (S)-22. FIG. 117D is a graph showing a linear correlation of the enantiomeric composition of 22 and the CD signal intensity generated upon formation of 45. CD measurements were taken in ACN at 0.45 mM.

FIGS. 118A-G show amino acid chirality sensing using probe 1 in aqueous solution. FIG. 118A shows the structures of the 19 chiral standard amino acids. FIGS. 118B-G show representative examples for amino acid chirality sensing using 1: aliphatic (FIG. 118B), aromatic (FIG. 118C), cyclic (FIG. 118D), hydrophilic (FIG. 118E), acidic (FIG. 118F), and basic sidechains (FIG. 118G). CD measurements were taken at 0.54 mM in ACN/borate buffer solutions.

FIGS. 119A-B show visualization of the unified CD sensing concept with six theoretical samples covering wide concentration and er ranges of a chiral target compound. FIG. 119A shows graphical analysis of a theoretical CD induction of three 0.84 mM samples with varying er (CD sensing of three 0.84 mM samples of A with 56.5:43.5, 81.85:18.15, and 94.55:5.45 er). FIG. 119B shows analysis of three samples with the same enantiomeric composition but different concentrations (CD sensing of three 81.05:18.95 er samples of A at 0.27, 0.43, and 0.93 mM).

FIGS. 120A-B show experimental determination of the absolute configuration, er, and concentration of selected amine 22 (FIG. 120A) and alanine (FIG. 120B) samples using the unified CD sensing concept. FIG. 120A shows graphical analysis of the sensing of samples containing amine 22 in various concentrations and enantiomeric compositions. The sample numbers 2, 5, 6, and 10 correspond to those shown in Table 4. FIG. 120B shows CD sensing results of four representative alanine samples. The sample numbers 1, 4, 7, and 10 correspond to those shown in Table 5. In both graphs, solid arrows indicate the x-axis intercepts providing the total concentration [(R)-22+(S)-22] or [D-Ala+L-Ala], hollow arrows show the y-axis intercept used to determine the er values. All sensing samples with excess of the R-amine (or D-Ala) appear above the y-axis and samples with excess of the S-enantiomer (or L-Ala) appear below the y-axis.

### DETAILED DESCRIPTION OF THE INVENTION

One aspect of the present application relates to an analytical method that includes the steps of: providing a sample potentially containing a chiral analyte that can exist in stereoisomeric forms; contacting the sample with a chromophore probe, wherein said contacting is carried out under conditions to permit binding of the chromophore probe to the chiral analyte, if present in the sample, to form a probe-labeled analyte; and detecting the probe-labeled analyte in the sample using a single chiroptical assay format, and determining the concentration of the analyte in the sample and one or both of (i) the absolute configuration of the analyte in the sample, and (ii) the enantiomeric and/or the diastereomeric composition of the analyte in the sample.

Depending on the probe design, both covalently binding probes and non-covalently binding probes can be used in the analytical method.

Another aspect of the present application relates to a kit for carrying out the analytical method described herein. The kit may include an aqueous or non-aqueous solution comprising a chromophore probe and, optionally, one or more of (i) sample tubes suitable for use with a spectrophotometer; (ii) an optically pure reference sample of an analyte, (iii) directions for using a spectrophotometer for carrying out circular dichroism (CD), vibrational CD (VCD), electronic CD, optical rotatory dispersion (ORD), or polarimetry analyses to measure the concentration of an analyte in a sample and one or both of the absolute configuration of the analyte in the sample, and the enantiomeric and/or the diastereomeric composition of the analyte in the sample, and (iv) a recordable medium comprising a template for analyzing data obtained from the spectrophotometer and determining the concentration of an analyte in a sample and one or both of the absolute configuration of the analyte in the sample, and the enantiomeric and/or the diastereomeric composition of the analyte in the sample.

A further aspect of the present application relates to the use of a chromophore probe as defined below in an analytical method of measuring the concentration of an analyte in a sample and one or both of the absolute configuration of the analyte in the sample, and the enantiomeric and/or the diastereomeric composition of the analyte in the sample.

### Probes

The probes of the present application include metal salts, quinones and analogs thereof, (hetero)aryl isocyanates and analogs thereof, (hetero)aryl isothiocyanates and analogs thereof, phenyl-naphthalene compounds and analogs thereof, aryl halophosphites and analogs thereof, aryl halodiazaphosphites and analogs thereof, coumarin-derived Michael acceptors and analogs thereof, dinitrofluoroarenes and analogs thereof, arylchlorophosphines and analogs thereof, metal complexed ligands, and various (hetero)arenesulfonyl compounds including arylsulfonyl halides.

Suitable metal salts are preferably those salts formed using type II transition metals and lanthanide metals that afford chiroptical signals at a high wavelength and/or at a high intensity, such as cobalt salts, palladium salts, copper salts, iron salts, manganese salts, cerium salts, and rhodium salts. A number of exemplary metal salts are disclosed in PCT Application Publ. No. WO 2020/056012, which is hereby incorporated by reference in its entirety.

Quinones are a class of organic compounds which possess a fully conjugated cyclic dione structure. A suitable class of quinone probes is disclosed in co-pending U.S. Provisional Patent Application Ser. No. 63/173,071, which is hereby incorporated by reference in its entirety. Exemplary quinones include, but are not limited to, 1,2-benzoquinone, 1,4-benzoquinone, 1,4-napthoquinone and 9,10-anthraquinone. An analog of a quinone is a quinone in which at least one of the hydrogen atoms has been replaced with a substituent including, but not limited to, a leaving group, a halogen, nitro, cyano, aryl, perfluoroaryl, heteroaryl, cycloalkyl, heterocycloalkyl, alkyl, or perfluoroalkyl.

A (hetero)aryl isocyanate is a (hetero)aryl that possesses at least one isocyanate (—N═C═O) bonded to the (hetero)aromatic ring. A suitable class of (hetero)aryl isocyanate probes is disclosed in co-pending U.S. Provisional Patent Application Ser. No. 63/173,071, which is hereby incorporated by reference in its entirety. An analog of a (hetero)aryl isocyanate is a (hetero)aryl isocyanate in which at least one of the hydrogens on the (hetero)aromatic ring has been replaced with a substituent including, but not limited to halogen, nitro, cyano, aryl, perfluoroaryl, heteroaryl, cycloalkyl, heterocycloalkyl, alkyl, or perfluoroalkyl.

A (hetero)aryl isothiocyanate is an is a (hetero)aryl that possesses at least one isothiocyanate (—N═C═S) bonded to the (hetero)aromatic ring. A suitable class of (hetero)aryl isothiocyanate probes is disclosed in U.S. Provisional Patent Application Ser. No. 63/173,071, which is hereby incorporated by reference in its entirety. An analog of a (hetero)aryl isothiocyanate is a (hetero)aryl isothocyanate in which at least one of the hydrogens on the (hetero)aromatic ring has been replaced with a substituent including, but not limited to, halogen, nitro, cyano, aryl, perfluoroaryl, heteroaryl, cycloalkyl, heterocycloalkyl, alkyl, or perfluoroalkyl.

A phenyl-naphthalene compound is an aryl compound that possesses a naphthalene core bearing a phenyl ring as a group on one of the naphthalene rings. A suitable class of phenyl-naphthalene probes is disclosed in U.S. Pat. No. 9,815,746, which is hereby incorporated by reference in its entirety. An analog of these phenyl-naphthalene compounds is a phenyl-naphthalene compound in which at least one of the hydrogens on the phenyl ring or the naphthalene core has been replaced with a substituent including, but not limited to, halogen, nitro, cyano, substituted or unsubstituted aryl, perfluoroaryl, substituted or unsubstituted heteroaryl, cycloalkyl, heterocycloalkyl, alkyl, or perfluoroalkyl.

An aryl halophosphite possesses one or more aryl rings coupled to a halophosphite group, preferably where the phosphite is integrated into a fused ring. A suitable class of aryl halophosphites is disclosed in PCT Application Publ. No. WO 2020/028396, which is hereby incorporated by reference in its entirety. An analog of these aryl halophosphites has at least one of the hydrogens on the aryl ring(s) replaced with a substituent including, but not limited to, halogen, nitro, cyano, aryl, perfluoroaryl, heteroaryl, cycloalkyl, heterocycloalkyl, alkyl, or perfluoroalkyl.

An aryl halodiazaphosphite possesses one or more aryl rings coupled to a halodiazaphosphite group, preferably where the diazaphosphite is formed into a fused ring. A suitable class of aryl halodiazaphosphites is disclosed in PCT Application Publ. No. WO 2020/028396, which is hereby incorporated by reference in its entirety. An analog of these aryl halodiazaphosphites has at least one of the hydrogens on the aryl ring(s) replaced with a substituent including, but not limited to, halogen, nitro, cyano, aryl, perfluoroaryl, heteroaryl, cycloalkyl, heterocycloalkyl, alkyl, or perfluoroalkyl.

A coumarin-derived Michael acceptor possesses a coumarin core having an electron withdrawing group. A suitable class of coumarin-derived Michael acceptors is disclosed in PCT Application Publ. No. WO 2020/028396, which is hereby incorporated by reference in its entirety. An analog of these coumarin-derived Michael acceptors has at least one of the hydrogens on the coumarin core replaced with a substituent including, but not limited to, halogen, nitro, cyano, aryl, perfluoroaryl, heteroaryl, cycloalkyl, heterocycloalkyl, alkyl, or perfluoroalkyl.

A dinitrofluoroarenes possess a phenyl ring bearing a fluoro group and a pair of nitro groups. A suitable class of dinitrofluoroarenes is disclosed in PCT Application Publ. No. W) 2020/028396, which is hereby incorporated by reference in its entirety. An analog of these dinitrofluoroarenes has at least one of the hydrogens on the phenyl ring replaced with a substituent including, but not limited to, halogen, nitro, cyano, aryl, perfluoroaryl, heteroaryl, cycloalkyl, heterocycloalkyl, alkyl, or perfluoroalkyl.

An arylchlorophosphine includes a chlorophosphine moiety spanning between two aryl groups. A suitable class of arylchlorophosphines is disclosed in PCT Application Publ. No. WO 2020/028396, which is hereby incorporated by reference in its entirety. An analog of these arylchlorophosphine has at least one of the hydrogens on the aryl rings replaced with a substituent including, but not limited to, halogen, nitro, cyano, aryl, perfluoroaryl, heteroaryl, cycloalkyl, heterocycloalkyl, alkyl, or perfluoroalkyl.

Metal complexed ligands include a metal and two or more ligands that include one or more aryl rings. A suitable class of metal complexed ligands is disclosed in U.S. Pat. No. 10,012,627 to Wolf et al., which is hereby incorporated by reference in its entirety. Analogs of these metal complexed ligands include those having at least one of the hydrogens on the aryl rings replaced with a substituent including, but not limited to, halogen, nitro, cyano, aryl, perfluoroaryl, heteroaryl, cycloalkyl, heterocycloalkyl, alkyl, or perfluoroalkyl.

Arenesulfonyl compounds and heteroarenesulfonyl compounds that are useful in practicing the disclosed method include achiral (hetero)arenesulfonyl compounds having the structure according to formula (I):

Ar—SO₂—Z (I),

wherein Ar is a substituted or unsubstituted aromatic or heteroaromatic chromophore, and Z is a leaving group. In certain embodiments, the leaving group is a halide (preferably chloride, bromide, or iodide), a phenolate, —O-aryl, —O-perfluoroaryl, —O-heteroaryl, —O-cycloalkyl, —O— heterocycloalkyl, —O-alkyl, or —O-perfluoroalkyl. Other suitable leaving groups can be selected by persons of skill in the art.

According to one embodiment, the probe is an achiral (hetero)arenesulfonyl compound of Formula Ia:

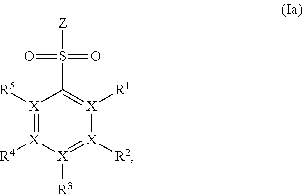

wherein.
each X is independently C or N, except that no more than three ring nitrogens are present in the (hetero)arenesulfonyl compound, and
R¹, R², R³, R⁴, and R⁵ are independently selected from the group consisting of a lone pair (when X is N), —H, —CN, —NO₂, halogen, —C₁-C₆ alkyl, —C₁-C₆ alkoxy, —N-(alkyl)₂, —C₁-C₆ alkenyl, —C₁-C₆ alkynyl, —C₁-C₆ perfluoroalkyl, -aryl, -perfluoroaryl, -aryloxy, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, —N-(heterocycloalkyl)₂, —C(O)Rₐ, —SO₂Rₐ, and —OC(O)Rₐ,;
each Rₐ is independently selected from the group consisting of -alkyl, —O-alkyl, —N-(alkyl)₂, -alkenyl, -alkynyl, -aryl, —O-aryl, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, and —N-(heterocycloalkyl)₂; and
wherein, optionally, R¹ and R², R² and R³, R³ and R⁴, and/or R⁴ and R⁵ are alternatively taken together with the carbon or nitrogen atoms to which they are attached to form a fused monocyclic or bicyclic ring system selected from the group consisting of cycloalkyl, heterocycloalkyl, aryl, and heteroaryl, wherein the fused ring system is optionally substituted with one or more groups selected from -alkyl, —O-alkyl, —N-(alkyl)₂, -alkenyl, -alkynyl, —O-aryl, —O-heteroaryl, —N-(aryl)₂, —N-(heteroaryl)₂, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

In certain embodiments, the probe according to formula Ia has one or two of R¹, R², R³, R⁴, and R⁵ that is —NO₂, —OMe, —NMe₂, or —OMe.

According to another embodiment, the probe is an achiral (hetero)arenesulfonyl compound according to Formula Ib:

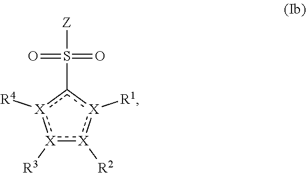

wherein:
each X is independently C or N; and
R¹, R², R³, and R⁴ are independently selected from the group consisting of —NCO, —NCS, a lone pair (when X is N), —H, —CN, —NO₂, halogen, —C₁-C₆ alkyl, —C₁-C₆ alkoxy, —N-(alkyl)₂, —C₁-C₆ alkenyl, —C₁-C₆ alkynyl, —C₁-C₆ perfluoroalkyl, -aryl, -perfluoroaryl, -aryloxy, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, —N-(heterocycloalkyl)₂, —OH, —C(O)Rₐ, —SO₂Rₐ, and —OC(O)Rₐ;
wherein each Rₐ is independently selected from the group consisting of —H, -alkyl, —O-alkyl, —N-(alkyl)₂, -alkenyl, -alkynyl, -aryl, —O-aryl, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, and —N-(heterocycloalkyl)₂; and
wherein, optionally, R¹ and R², R² and R³, and/or R³ and R⁴ is alternatively taken together with the carbon atoms to which they are attached to form a fused monocyclic or bicyclic ring system selected from the group consisting of cycloalkyl, heterocycloalkyl, aryl, and heteroaryl, wherein the fused ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, -—NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

In certain embodiments, the probe according to formula Ib has one or two of R¹, R², R³, and R⁴ that is —NO₂, —OMe, —NMe₂, or —OMe.

Exemplary achiral (hetero)arenesulfonyl probes include, without limitation:

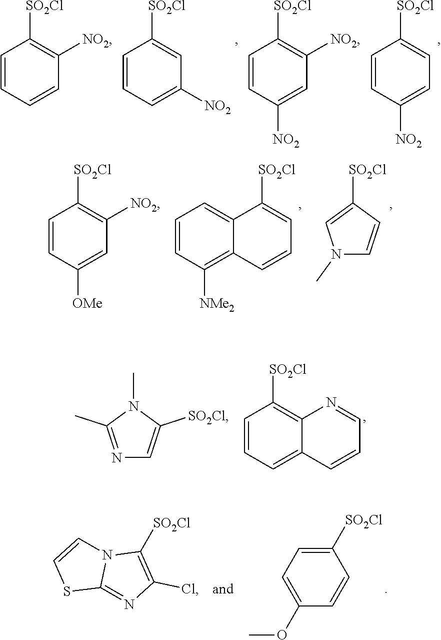

Additional arylsulfonyl chloride probes that useful in practicing the disclosed analytical methods include those described in PCT Application Publ. No. WO 2020/028396, which is hereby incorporated by reference in its entirety.

As used herein, the term “alkyl” refers to a straight or branched, saturated aliphatic radical containing one to about twenty (e.g., 1, 2, 3, 4, 5, 6, 7, 8, 9, 10, 11, 12, 13, 14, 15, 16, 17, 18, 19, 20, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 14-18, 14-19, 14-20, 15-16, 15-17, 15-18, 15-19, 15-20, 16-17, 16-18, 16-19, 16-20, 17-18, 17-19, 17-20, 18-19, 18-20, 19-20) carbon atoms and, unless otherwise indicated, may be optionally substituted. In at least one embodiment, the alkyl is a C₁-C₁₀ alkyl. In at least one embodiment, the alkyl is a C₁-C₆ alkyl. Suitable examples include, without limitation, methyl, ethyl, propyl, isopropyl, butyl, sec-butyl, isobutyl, tert-butyl, 3-pentyl, and the like.

As used herein, the term “alkenyl” refers to a straight or branched aliphatic unsaturated hydrocarbon of formula CₙH₂ₙ having from two to about twenty (e.g., 2, 3, 4, 5, 6, 7, 8, 9, 10, 11, 12, 13, 14, 15, 16, 17, 18, 19, 20, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 14-18, 14-19, 14-20, 15-16, 15-17, 15-18, 15-19, 15-20, 16-17, 16-18, 16-19, 16-20, 17-18, 17-19, 17-20, 18-19, 18-20, 19-20) carbon atoms in the chain and, unless otherwise indicated, may be optionally substituted. Exemplary alkenyls include, without limitation, ethylenyl, propylenyl, n-butylenyl, and i-butylenyl.

As used herein, the term “alkynyl” refers to a straight or branched aliphatic unsaturated hydrocarbon of formula CₙH₂ₙ₋₂ having from two to about twenty (e.g., 2, 3, 4, 5, 6, 7, 8, 9, 10, 11, 12, 13, 14, 15, 16, 17, 18, 19, 20, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 14-18, 14-19, 14-20, 15-16, 15-17, 15-18, 15-19, 15-20, 16-17, 16-18, 16-19, 16-20, 17-18, 17-19, 17-20, 18-19, 18-20, 19-20) carbon atoms in the chain and, unless otherwise indicated, may be optionally substituted. Exemplary alkynyls include acetylenyl, propynyl, butynyl, 2-butynyl, 3-methylbutynyl, and pentynyl.

As used herein, the term “cycloalkyl” refers to a non-aromatic saturated or unsaturated monocyclic or polycyclic (e.g., bicyclyic, tricyclic, tetracyclic) ring system which may contain 3 to 24 (3, 4, 5, 6, 7, 8, 9, 10, 11, 12, 13, 14, 15, 16, 17, 18, 19, 20, 21, 22, 23, 24, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 7-8, 7-9, 7-10, 7-11, 7-12, 7-13, 7-14, 7-15, 7-16, 7-17, 7-18, 7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 22-23, 22-24, 23-24) carbon atoms, which may include at least one double bond and, unless otherwise indicated, the ring system may be optionally substituted. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, and syn-bicyclopropane.

As used herein, the term “heterocycloalkyl” refers to a cycloalkyl group as defined above having at least one O, S, and/or N interrupting the carbocyclic ring structure. Examples of heterocycloalkyls include, without limitation, piperidine, piperazine, morpholine, thiomorpholine, pyrrolidine, tetrahydrofuran, pyran, tetrahydropyran, and oxetane. Unless otherwise indicated, the heterocycloalkyl ring system may be optionally substituted.

As used herein, the term “aryl” refers to an aromatic monocyclic or polycyclic (e.g., bicyclyic, tricyclic, tetracyclic) ring system from 6 to 24 (6, 7, 8, 9, 10, 11, 12, 13, 14, 15, 16, 17, 18, 19, 20, 21, 22, 23, 24, 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 7-8, 7-9, 7-10, 7-11, 7-12, 7-13, 7-14, 7-15, 7-16, 7-17, 7-18, 7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 22-23, 22-24, 23-24) carbon atoms and, unless otherwise indicated, the ring system may be optionally substituted. Aryl groups of the present technology include, but are not limited to, groups such as phenyl, naphthyl, azulenyl, phenanthrenyl, anthracenyl, fluorenyl, pyrenyl, triphenylenyl, chrysenyl, naphthacenyl, biphenyl, triphenyl, and tetraphenyl. In at least one embodiment, an aryl within the context of the present technology is a 6 or 10 membered ring. In at least one embodiment, each aryl is phenyl or naphthyl.

As used herein, the term “heteroaryl” refers to an aryl group as defined above having at least one O, S, and/or N interrupting the carbocyclic ring structure. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl. Additional heteroaryls are described in COMPREHENSIVE HETEROCYCLIC CHEMISTRY: THE STRUCTURE, REACTIONS, SYNTHESIS AND USE OF HETEROCYCLIC COMPOUNDS (Katritzky et al. eds., 1984), which is hereby incorporated by reference in its entirety. Unless otherwise indicated, the heteroaryl ring system may be optionally substituted.

As used herein, the terms “alkoxy” refers to a groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

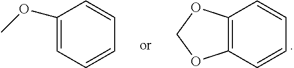

As used herein, the term “aryloxy” refers to OR, where R is an aryl group.

As used herein, the terms “perfluoroalkyl”, “perfluoroalkenyl”, “perfluoroalkynyl”, and “perfluoroaryl” refer to an alkyl, alkenyl, alkynyl, or aryl group as defined above in which the hydrogen atoms on at least one of the carbon atoms have all been replaced with fluorine atoms.

The term “monocyclic” as used herein indicates a molecular structure having one ring.

The term “polycyclic” as used herein indicates a molecular structure having two or more rings, including, but not limited to, fused, bridged, spiro, or covalently bound rings. In at least one embodiment, the polycyclic ring system is a bicyclic, tricyclic, or tetracyclic ring system. In at least one embodiment, the polycyclic ring system is fused. In at least one embodiment, the polycyclic ring system is a bicyclic ring system such as naphthyl or biphenyl.

As used herein, the term “optionally substituted” indicates that a group may have a substituent at each substitutable atom of the group (including more than one substituent on a single atom), provided that the designated atom's normal valency is not exceeded and the identity of each substituent is independent of the others. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” is meant a compound that is sufficiently robust to survive isolation to a useful degree of purity from a reaction mixture, and formulation into an efficacious agent.

As used herein, the term “unsubstituted” means that atoms bear all of the hydrogen atoms dictated by their valency.

As used herein, the term “halogen” includes fluorine, bromine, chlorine, and iodine.

As used herein, “leaving groups” are substituents that are present on the compound that can be displaced. Although several preferred leaving groups are identified above, a number of other suitable leaving groups will be apparent to a skilled artisan.

As used herein, an “aromatic or heteroaromatic chromophore” refers to an aromatic or heteroaromatic group that produces a signal that can be used for chiroptical detection through various approaches including, without limitation, circular dichroism (CD), vibrational CD (VCD), electronic CD, optical rotatory dispersion (ORD), or polarimetry.

A UV chromophore shows a good absorption behavior in the spectral range of the UV rays or preferably an absorption maximum above 250 nm. The chromophore absorbs the energy of the ultraviolet light and preferably does not change chemically as a result. The energy can be released as heat or phosphorescence/fluorescence. Visible chromophores include compounds with absorption from about 380 nm to 740 nm, which absorb light in the visible spectrum. UV/Vis chromophores have a conjugated pi system, such as those found in aromatic compounds.

### Analytes

The analytical methods described herein may be used to evaluate a wide range of chiral analytes. The analyte is one that can exist in stereoisomeric forms. This includes enantiomers, diastereomers, and a combination thereof.

Exemplary chiral analytes and the classes of probes that can discriminate those analytes using the chiroptical analytical methods as disclosed herein are shown in the Table 1 below.

TABLE 1
List of Probes and Chiral Analytes

| Probe | Chiral Analyte |
|---|---|
| Metal salts | amino acids, a-hydroxy acids, amino |
| | phosphonic acids, amino alcohols, and amines; |
| Substituted phenyl- | monoamines, amino alcohols, amino acids, and |
| naphthalene | combinations thereof; |
| aryl halophosphite | primary amines, secondary amines, amino |
| | alcohols, alcohols, hydroxy acids, amino acids, |
| | and combinations thereof; |
| halodiazaphosphite | primary amines, secondary amines, amino |
| | alcohols, alcohols, hydroxy acids, amino acids, |
| | and combinations thereof; |
| coumarin-derived | primary amines, secondary amines, amino |
| Michael acceptor | alcohols, alcohols, hydroxy acids, amino acids, |
| | and combinations thereof; |
| dinitrofluoroarene | primary amines, secondary amines, amino |
| | alcohols, alcohols, hydroxy acids, amino acids, |
| | and combinations thereof; |
| arylchlorophosphine | primary amines, secondary amines, amino |
| | alcohols, alcohols, hydroxy acids, amino acids, |
| | and combinations thereof; |
| an achiral quinone | amines, amino acids, amino alcohols, and |
| | combinations thereof; |
| (hetero)aryl isocyanate | amines, amino acids, amino alcohols, diols, |
| | alcohols, and combinations thereof; |
| (hetero)aryl | amines, amino acids, amino alcohols, diols, |
| isothiocyanate | alcohols, and combinations thereof; |
| (hetero)arenesulfonyl | primary amines, secondary amines, amino |
| compound | alcohols, amino acids, and combinations thereof; |
| Metal complexed | amines, diamines, a-hydroxy acids, amino acids, |
| ligands | and amino alcohols. |

### Reaction Conditions

In at least one embodiment of any analytical method herein, contacting is carried out for about 1 to about 300 minutes (e.g., carried out for a duration range having an upper limit of about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, or about 300 minutes, and a lower limit of about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, or about 290 minutes, or any combination thereof). In all embodiments, contacting is carried out for a time that is sufficient for the probe to bind to any analyte present in the sample. As will be apparent to the skilled chemist, the speed at which binding takes place will depend on various factors, including the particular probe selected and the analyte, whether a catalyst is present, concentrations, and the temperature.

As will be apparent to the skilled chemist, the analytical methods may be carried out at room temperature, at high temperatures (e.g., about 50° C. to about 100° C., e.g., a temperature range with an upper limit of about 55° C., about 60° C., about 65° C., about 70° C., about 75° C., about 80° C., about 85° C., about 90° C., about 95° C., or about 100° C., and a lower limit of about 50° C., about 55° C., about 60° C., about 65° C., about 70° C., about 75° C., about 80° C., about 85° C., about 90° C., or about 95° C., or any combination thereof), or at low temperatures (e.g., below about 25° C., e.g., below about 25° C., below about 20° C., below about 15° C., below about 10° C., below about 5° C., below about 0° C., below about −5° C., below about −10° C., below about −15° C., below about −20° C., below about −25° C., below about −30° C., below about −35° C., below about −40° C., below about −45° C., below about −50° C., below about −55° C., below about −60° C., below about −65° C., below about −70° C., or below about −75° C., preferably no lower than about −78° C.; e.g., a temperature range with an upper limit of about 25° C., about 20° C., about 15° C., about 10° C., about 5° C., about 0° C., about −5° C., about −10° C., about −15° C., about −20° C., about −25° C., about −30° C., about −35° C., about −40° C., about −45° C., about −50° C., about −55° C., about −60° C., about −65° C., about −70° C., or about −75° C., and a lower limit of about 20° C., about 15° C., about 10° C., about 5° C., about 0° C., about −5° C., about −10° C., about −15° C., about −20° C., about −25° C., about −30° C., about −35° C., about −40° C., about −45° C., about −50° C., about −55° C., about −60° C., about −65° C., about −70° C., about −75° C., or about −78° C., or any combination thereof). Furthermore, the analytical methods may be carried out under ambient conditions (e.g., 23±3° C. and 38±5% relative humidity).

For example, the temperature could be increased to speed up the binding reaction. Some analyte-probe combinations may have side reactions at certain temperatures; the temperature could be decreased to prevent such side reactions.

The analytical methods could also optionally be carried out in the presence of a base. The use of a base may be helpful when the analyte is an acid (e.g., a carboxylic acid) or when an acid may be generated in situ. Adding an equivalent of base could also be helpful to avoid side reactions. Suitable bases include both organic and inorganic bases (or mixtures thereof). Exemplary bases include, but are not limited to: alkoxides such as sodium tert-butoxide; alkali metal amides such as sodium amide, lithium diisopropylamide, and alkali metal bis(trialkylsilyl)amide, e.g., such as lithium bis(trimethylsilyl)amide (LiHMDS) or sodium bis(trimethylsilyl)amide (NaHMDS); tertiary amines (e.g. triethylamine, trimethylamine, 4-(dimethylamino)pyridine (DMAP), 1,5-diazabicycl[4.3.0]non-5-ene (DBN), 1,5-diazabicyclo[5.4.0]undec-5-ene (DBU); alkali or alkaline earth carbonate, bicarbonate or hydroxide (e.g. sodium, magnesium, calcium, barium, potassium carbonate, phosphate, hydroxide and bicarbonate); and ammonium hydroxides, e.g. tetrabutylammonium hydroxide (TBAOH).

The analytical methods could also optionally be carried out in the presence of a buffer. Exemplary buffers include, but are not limited to, borate, phosphate, carbonate, Trizma, and Hepes buffers between pH 2-12.

In certain embodiments, the contacting step is carried out in a solvent selected from aqueous solvents, protic solvents, aprotic solvents, and any combination thereof. Exemplary solvents include, but are not limited to, chloroform, dichloromethane, acetonitrile, toluene, tetrahydrofuran, methanol, ethanol, isopropanol, water, dimethyl sulfoxide (DMSO), dimethylformamide (DMF), pentane, pentane isomers, hexane, hexane isomers, ether, dichloroethane, acetone, ethyl acetate, butanone, and mixtures of any combination thereof.

In certain embodiments, the analytical methods are carried out under aerobic conditions (e.g., under air or in an aqueous environment).

### Analysis

In the analytical methods described herein, the probe is reacted with the analyte to form probe-analyte complexes through either a covalent bond or non-covalent bond between the probe and the analyte. The probe-analyte complexes generate a chiroptical signal that can be used to determine the concentration of the analyte in the sample and one or both of (i) the absolute configuration of the analyte in the sample, and (ii) the enantiomeric and/or the diastereomeric composition of the analyte in the sample.

Importantly, and in contrast with prior approaches for the assessment of the concentration of the analyte in the sample, the absolute configuration of the analyte in the sample, and the enantiomeric and/or the diastereomeric composition of the analyte in the sample—which utilized multiple chiroptical detection procedures and instruments—in accordance with the present invention only a single chiroptical assay format is performed. The chiroptical signal of the probe-analyte complexes can be measured using standard techniques, which will be apparent to the skilled artisan. Such techniques include circular dichroism spectroscopy as well as the related vibrational circular dichroism and electronic circular dichroism spectroscopy formats (e.g., STEREOCHEMISTRY OF ORGANIC COMPOUNDS 1003-07 (E. L. Eliel & S. H. Wilen eds., 1994); DYNAMIC STEREOCHEMISTRY OF CHIRAL COMPOUNDS 140-43 (Christian Wolf ed., 2008), each of which is hereby incorporated by reference in its entirety), optical rotatory dispersion (e.g., STEREOCHEMISTRY OF ORGANIC COMPOUNDS 999-1003 (E. L. Eliel & S. H. Wilen eds., 1994), which is hereby incorporated by reference in its entirety), and polarimetry (e.g., STEREOCHEMISTRY OF ORGANIC COMPOUNDS 217-21, 1071-80 (E. L. Eliel & S. H. Wilen eds., 1994); DYNAMIC STEREOCHEMISTRY OF CHIRAL COMPOUNDS 140-43 (Christian Wolf ed., 2008), each of which is hereby incorporated by reference in its entirety). The absolute configuration of the analyte can also be assigned from the chiroptical signal of the probe-analyte complexes that form. The configuration assignment can be based on the sense of chirality induction with a reference or by analogy.

The term “enantiomeric composition” refers to the enantiomeric ratio and/or enantiomeric excess of an analyte. The enantiomeric ratio (er) is the ratio of the percentage of one analyte enantiomer in a mixture to that of the other enantiomer. The enantiomeric excess (ee) is the difference between the percentage of one analyte enantiomer and the percentage of the other analyte enantiomer. For example, a sample which contains 75% L-analyte and 25% D-analyte will have an enantiomeric excess of 50% of L-analyte and an enantiomeric ratio (D:L) of 25:75.

The term “diastereomeric composition” refers to the diastereomeric ratio and/or diastereomeric excess of an analyte. The diastereomeric ratio is the ratio of the percentage of one analyte diastereomer in a mixture to that of the other diastereomer. The diastereomeric excess is the difference between the percentage of one analyte diastereomer and the percentage of the other analyte diastereomer. For example, a sample which contains 75% R,S-analyte and 25% S,S-analyte will have a diastereomeric excess of 50% of R,S-analyte and a diastereomeric ratio (S,S:R,S) of 25:75.

In carrying out the analytical method, the step of contacting the sample with a chromophore probe to form a probe-labeled analyte is carried out on at least three measurements to which different known concentrations of the chromophore probe are introduced. One of the at least three measurements comprises an excess concentration of the chromophore probe, i.e., a saturating concentration that represents a maximum signal that can be detected in the chiroptical assay format used for detection. In certain embodiment, the at least three measurements can be carried out using four or more measurements, five or more measurements, six or more measurements, seven or more measurements, eight or more measurements, nine or more measurements, or ten or more measurements. In general, the greater the number of measurements, then the more accurate the assessment of the enantiomeric and/or the diastereomeric composition of the analyte in the sample.

In determining the concentration of the analyte in the sample and one or both of (i) the absolute configuration of the analyte in the sample, and (ii) the enantiomeric and/or the diastereomeric composition of the analyte in the sample, the intensity measurements obtained from the chiroptical assay (y-axis) are plotted against the chromophore probe concentration (x-axis) for the at least three measurements, and the plotted data are then analyzed using a linear regression analysis.

As demonstrated in the accompanying examples, the concentration of the analyte in the sample corresponds to the x-axis value at the intersection of the plotted regression line and a horizontal line (slope=0) representing the maximum intensity from the measurement comprising the excess concentration of the chromophore probe.

As demonstrated in the accompanying examples, the enantiomeric and/or the diastereomeric composition of the analyte in the sample is calculated by comparing the y-axis value at the intersection of the regression line and the horizontal line (slope=0) representing the maximum intensity with a value one would get for an enantiopure reference at the same concentration. Based on these results, it becomes possible to calculate one or both of an enantiomeric ratio (er) for the analyte and an enantiomeric excess (ee) for a major enantiomer of the analyte. For example:

$$\% ee = \frac{y - axis value [ mdeg ] \times 100}{mdeg value of enantiopure {reference}^{*}}$$

$${}^{*} enantiopure reference at same concentration and same solvent$$

The relationship between the er and ee is discussed above, and based upon the determination of the ee for the major enantiomer it is possible to determine the concentration of both enantiomers and, thus, the er.

The analytical methods of the present application provide, among other things, rapid and convenient tools for simultaneously determining the concentration as well as the enantiomeric composition and/or the diastereomeric composition and/or absolute configuration of chiral analytes while using a single assay format. These analytical methods may be particularly useful, for example, for evaluating high-throughput reactions whose desired product is chiral. For example, the present methods can be used to determine the enantiomeric/diastereomeric composition of the desired product, thus indicating the stereoselectivity of the reaction. Similarly, the present methods can be used to determine the concentration of the total product and/or the desired isomer, thus indicating the overall or individual yield of the reaction. Importantly, this can be carried out without the need to perform separate chiroptical assay formats—one for assessing concentration and another for assessing the enantiomeric/diastereomeric composition.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The present technology may be further illustrated by reference to the following examples.

### Examples

The examples below are intended to exemplify the practice of embodiments of the disclosure but are by no means intended to limit the scope thereof.

### Materials and Methods

All reagents and solvents were commercially available and used without further purification. Reactions were carried out at room temperature under anhydrous conditions. Flash chromatography was performed on silica gel, particle size 40-63 m. ¹H NMR spectra were obtained at 400.0 MHz using deuterated acetonitrile or chloroform as solvents. Chemical shifts were reported in ppm relative to TMS or to the solvent peak.

### Example 1—Probe Development and Optimization Studies

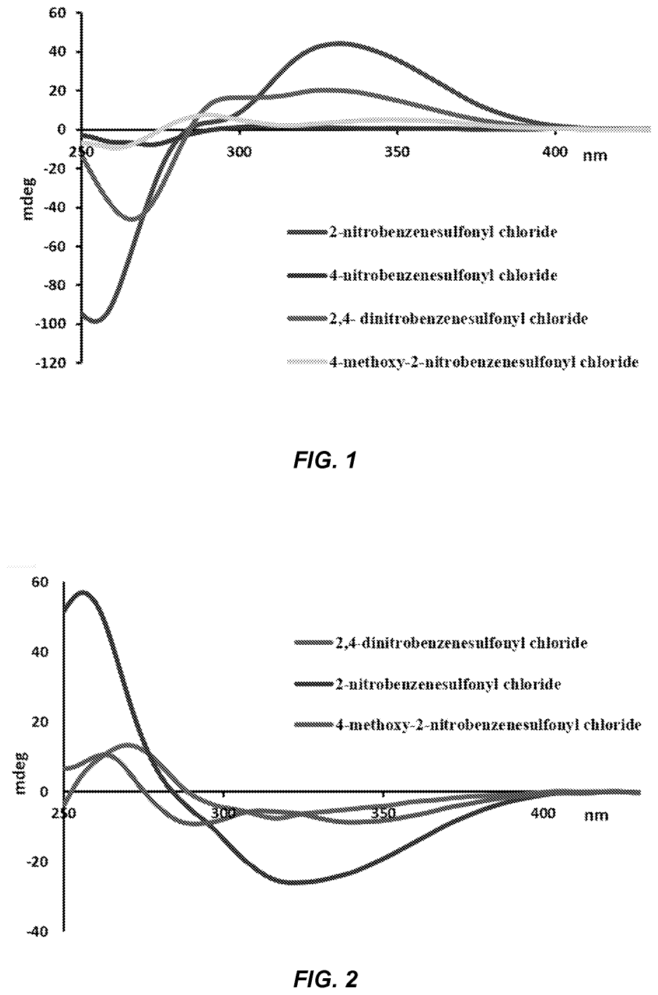

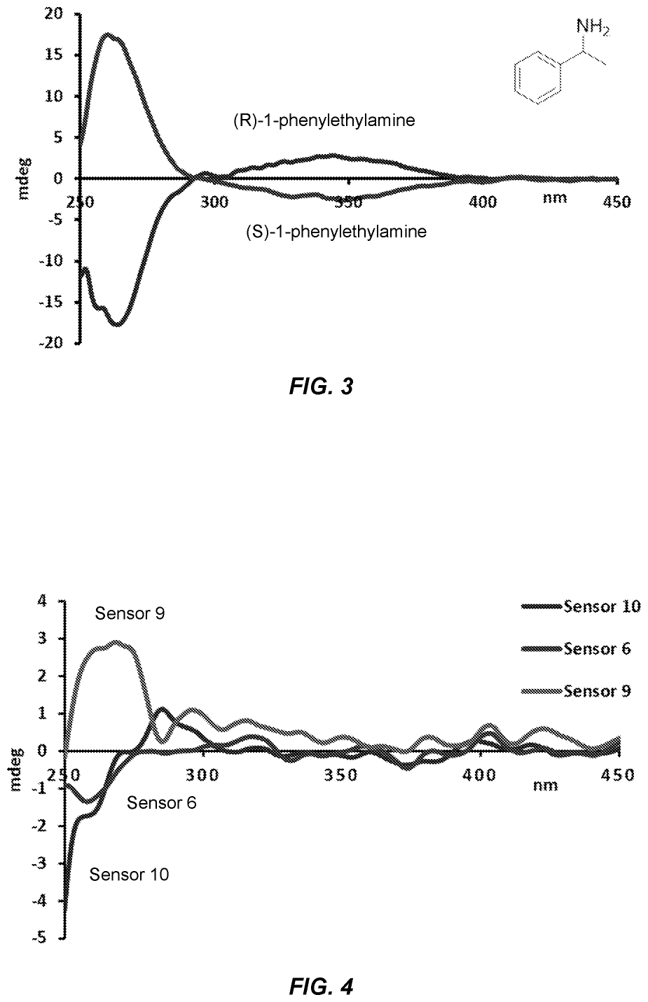

A solution containing probe 1 (50.0 mM in ACN, 480.0 μL), a chiral amine (50.0 mM in ACN, 400.0 μL), and Et₃N (1.2 eq) was stirred for 1 hour. CD measurements were taken by diluting 35.0 μL of this mixture with 2.0 mL of acetonitrile. Control experiments with (R)-1-phenylethylamine in the absence of the probe did not show any CD signal at the wavelengths of interest. The analysis was repeated with probes 2, 5, 6, 9, and 10 in ACN, probe 3 in THF, and probe 4 in chloroform (FIGS. 1-4) (Scheme 1). Probes 7 and 8 were insufficiently soluble in DMSO, acetonitrile, and chloroform. Among the ten arylsulfonyl chloride sensors tested, 2-nitrobenzenesulfonyl chloride 1 generated the largest CD amplitudes.

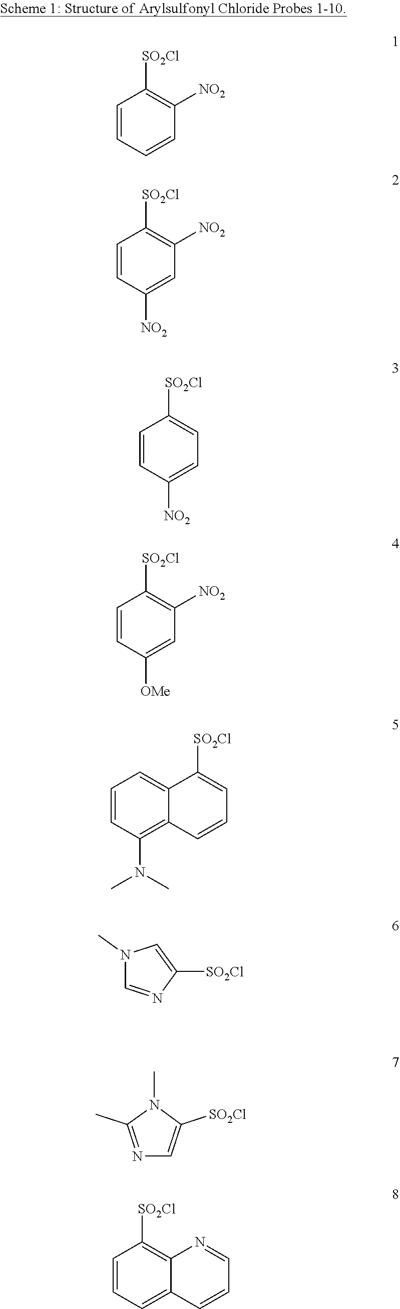

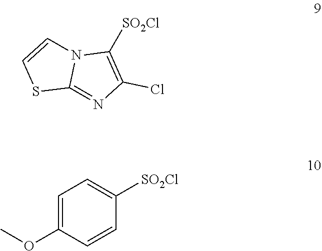

### Example 2—Mechanistic Studies of Probe 1 Reaction with Amines & Amino Acids by NMR Analysis, CD Analysis, and MS Analysis

### NMR Analysis

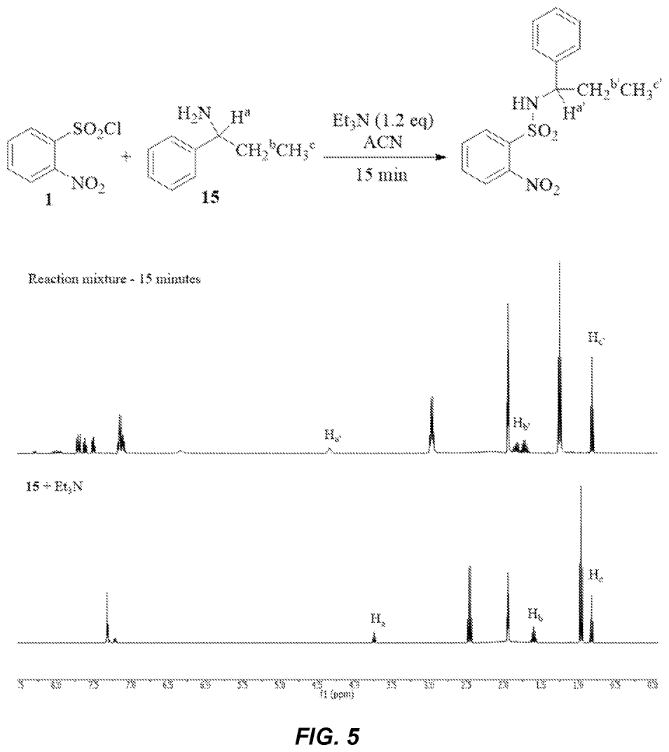

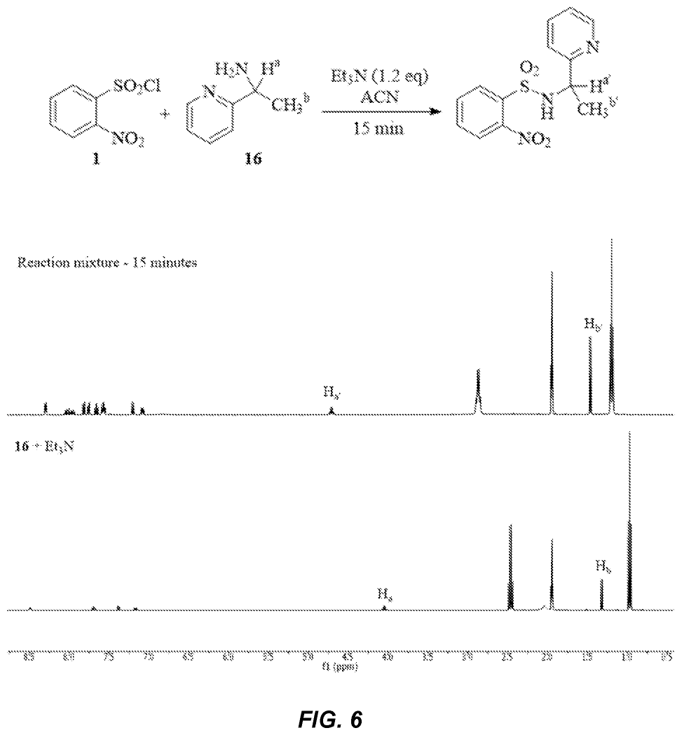

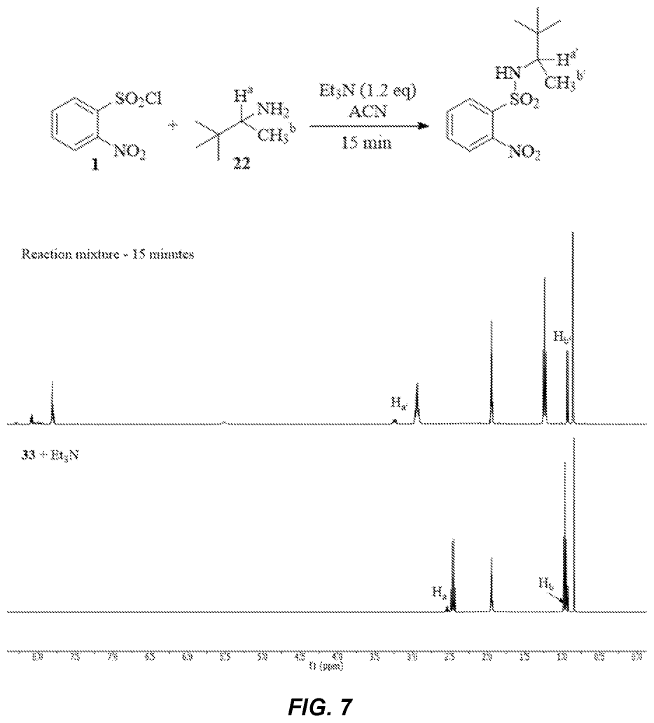

A solution prepared from probe 1 (0.1 M in d-ACN, 100.0 μL), amine 15 (0.1 M in d-ACN, 80.0 μL), and Et₃N (1.2 equiv.) was diluted to 1.0 mL using d₃-ACN. The reaction mixture was stirred for 15 minutes and subjected to ¹H NMR analysis (FIG. 5). The same procedure was performed with amines 16 and 33 (FIGS. 6-7). Upon sulfonamide formation with probe 1, the methine proton Ha in 15, 16, and 33 shifted from 3.74 to 4.34, 4.05 to 4.71, and 2.54 to 3.24 ppm, respectively. The conversion was quantitative and there was no sign of a side reaction.

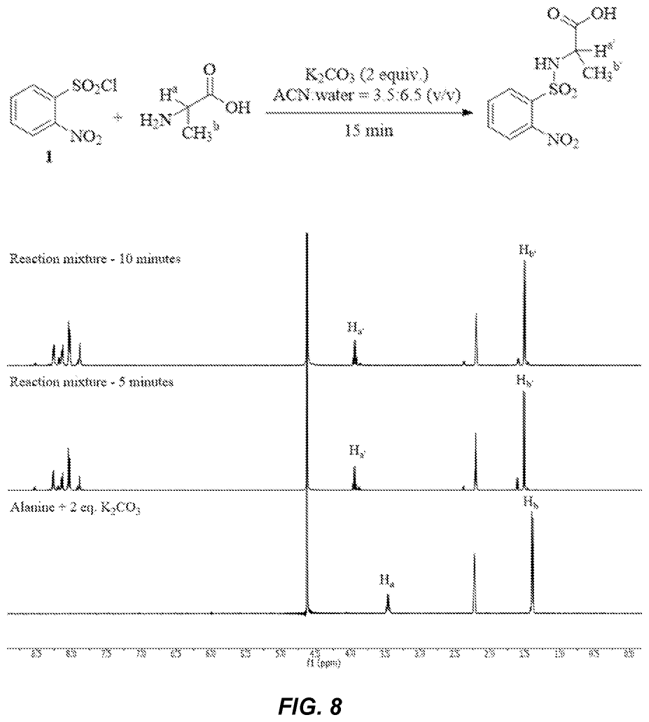

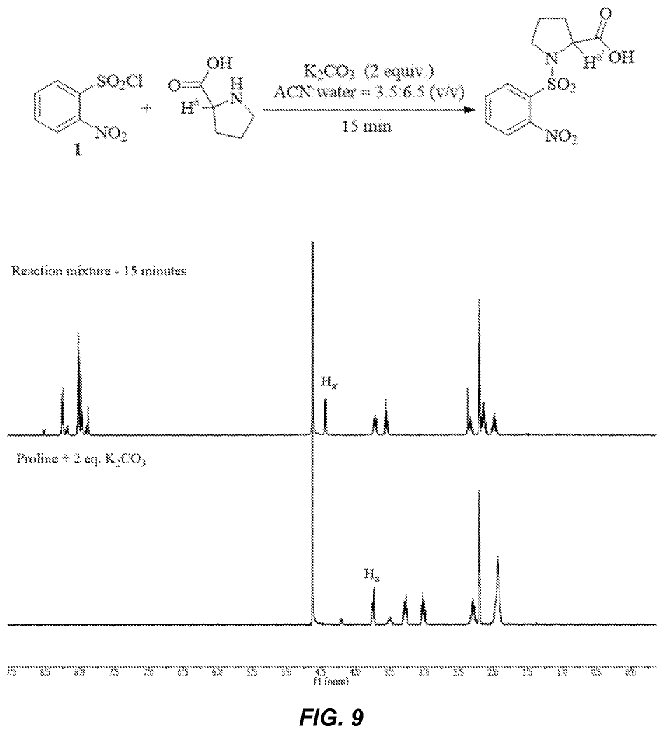

A solution prepared from probe 1 (0.1 M in d-ACN, 100.0 μL), alanine (0.1 M in D₂O, 80.0 μL), and K₂CO₃ (2.0 equiv.) was diluted to 1.0 mL using D₂O:d₃-ACN (6.5:3.5 v v). The reaction mixture was stirred for 15 minutes and subjected to ¹H NMR analysis (FIG. 8). The same procedure was performed with proline (FIG. 9). Upon sulfonamide formation with probe 1, the methine proton Ha in alanine and proline shifted from 3.47 to 3.95 and 3.75 to 4.44 ppm, respectively. The conversion was quantitative and there was no sign of a side reaction.

### CD Analysis

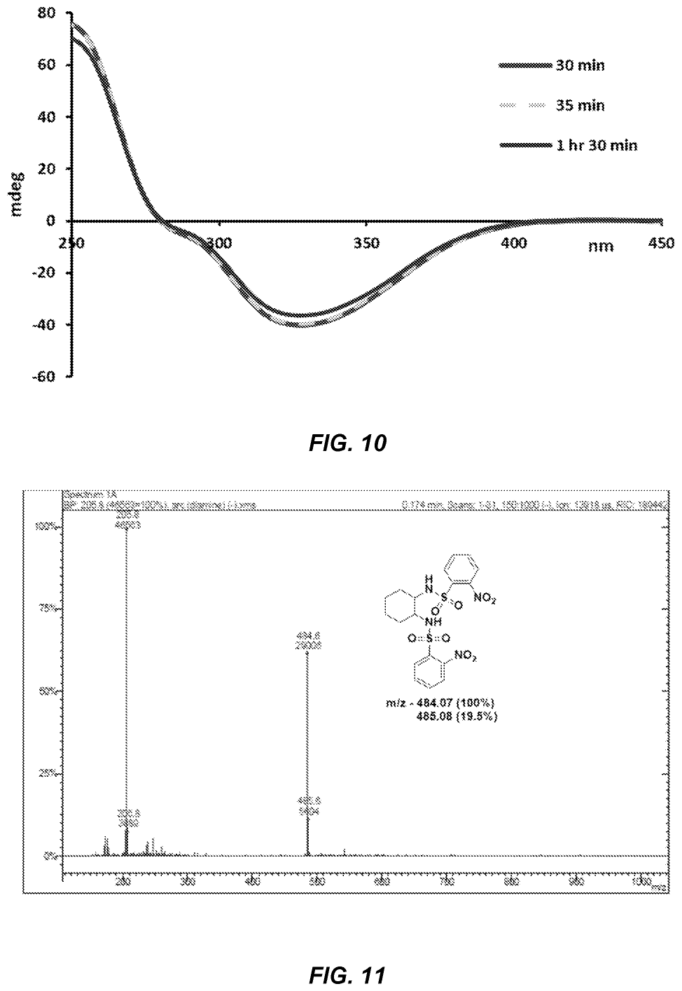

Solutions of (R)-serine (25.0 mM in pH 8.5 sodium borate buffer (250.0 mM), 400.0 μL) and probe 1 (25.0 mM in ACN, 480.0 μL) were combined and the reaction was monitored using CD spectroscopy. CD spectra were taken after diluting 30.0 μL of the mixture with 2.0 mL of CAN (FIG. 10). The reaction was complete within 30 minutes under these conditions.

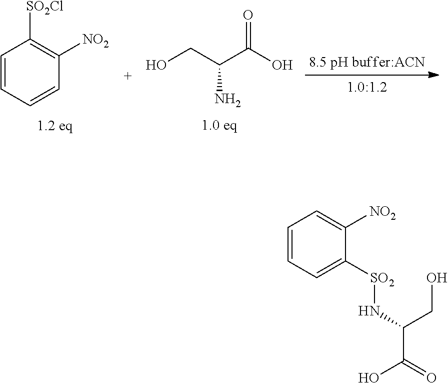 [SMILES: O=[N+]([O-])c1ccccc1S(=O)(=O)Cl ; N[C@H](CO)C(=O)O ; O=C(O)[C@@H](CO)NS(=O)(=O)c1ccccc1[N+](=O)[O-]]

### MS Analysis

ESI-MS analysis of the reaction between (R,R)-diaminocyclohexane (50.0 mM in ACN, 400.0 μL) and probe 1 (100.0 mM in ACN, 480.0 μL) in the presence of 2.4 equivalents of Et₃N was performed. After 30 minutes, an 8.0 μL aliquot of the reaction mixture was diluted to 2.0 mL with ACN for ESI-MS analysis (FIG. 11).

### Example 3—Sensing of Amines and Amino Alcohols by CD Analysis

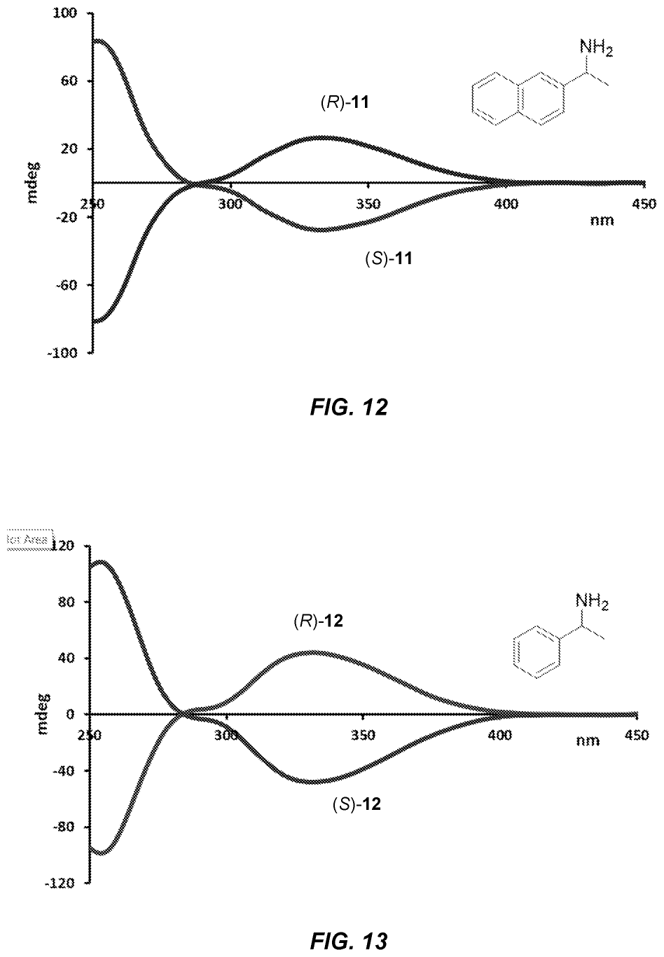

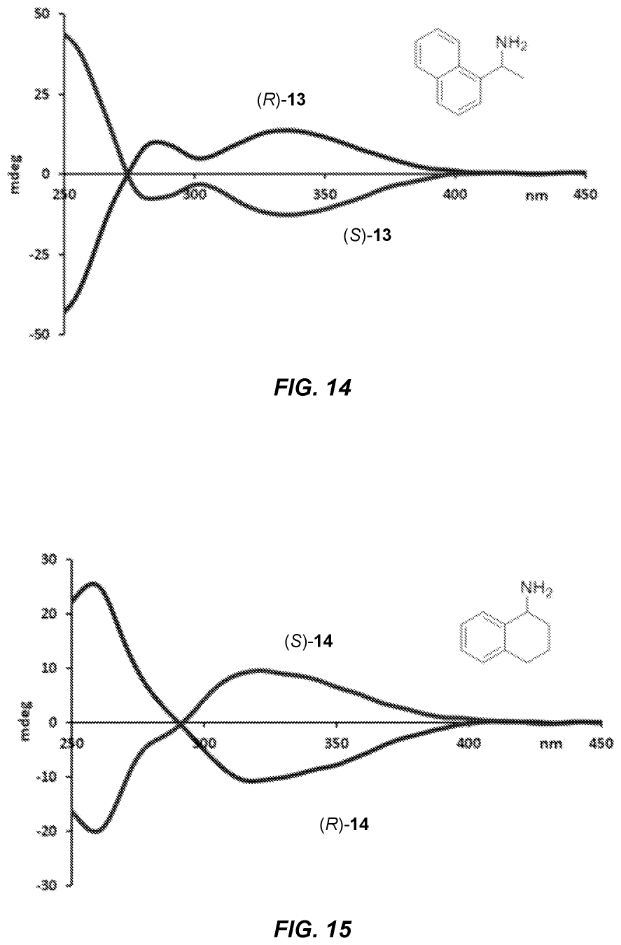

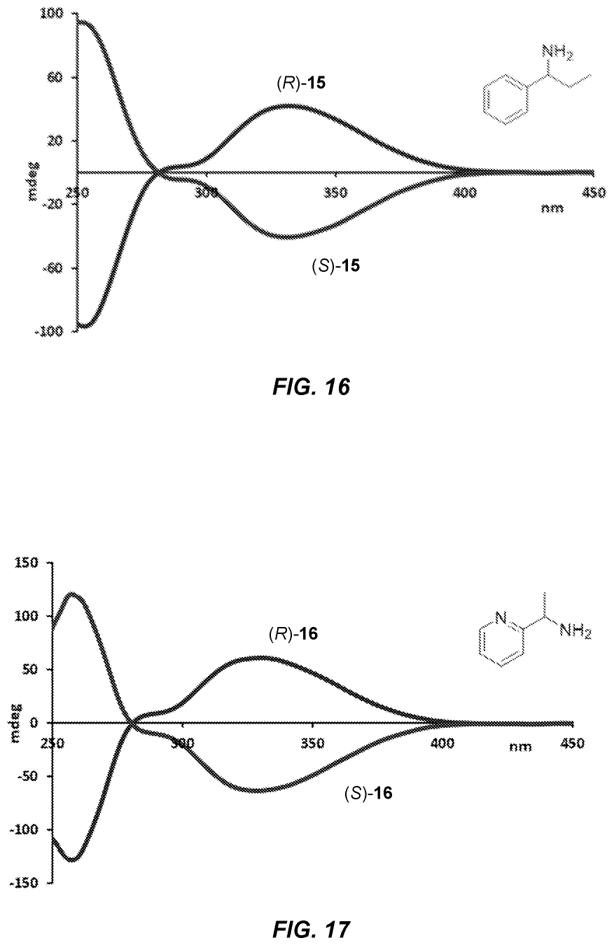

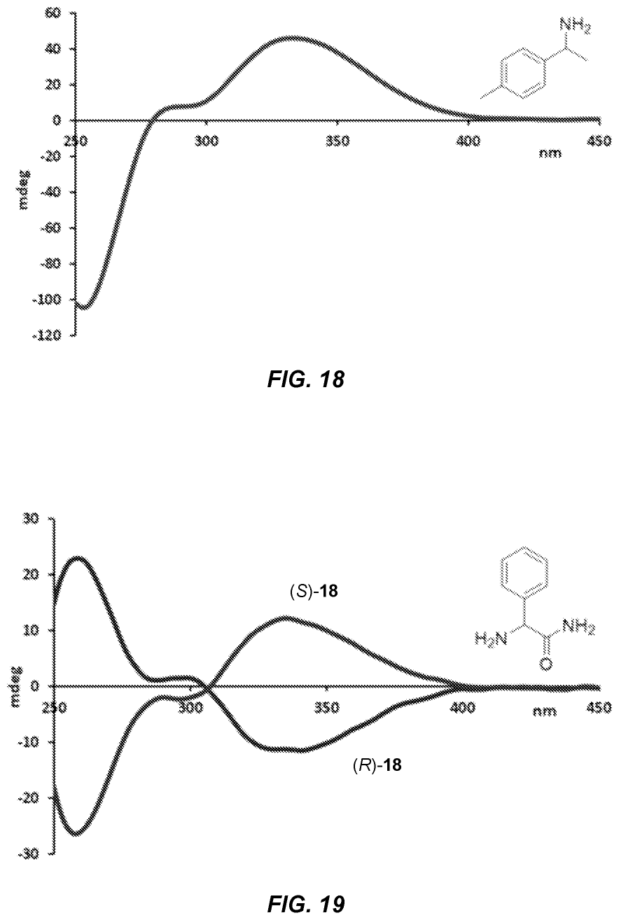

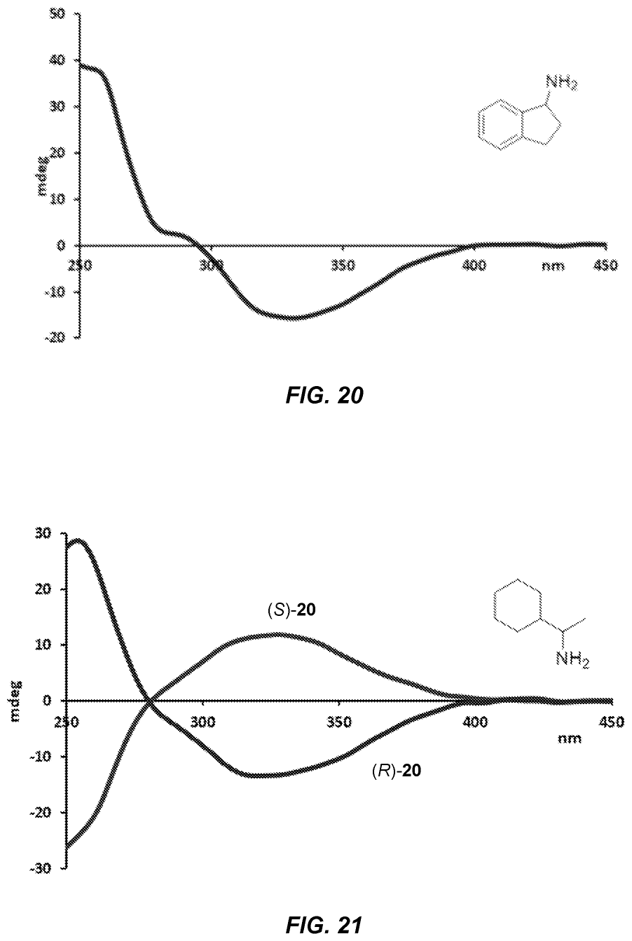

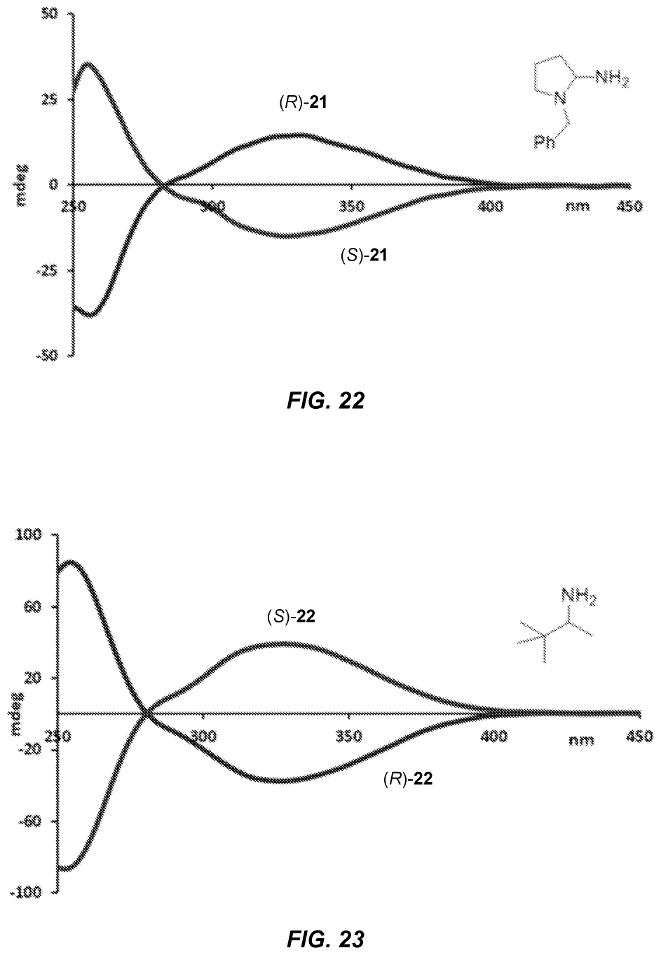

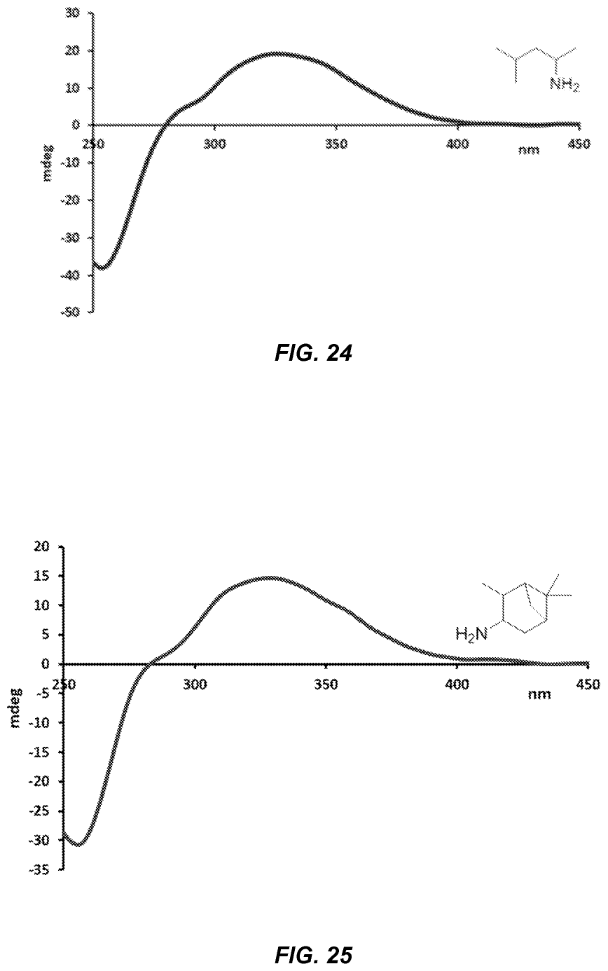

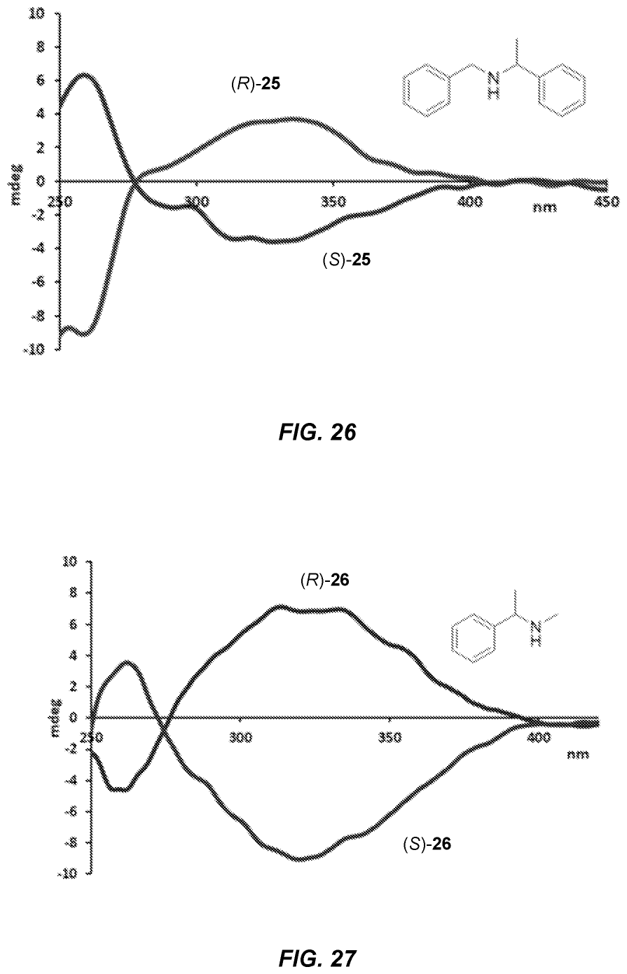

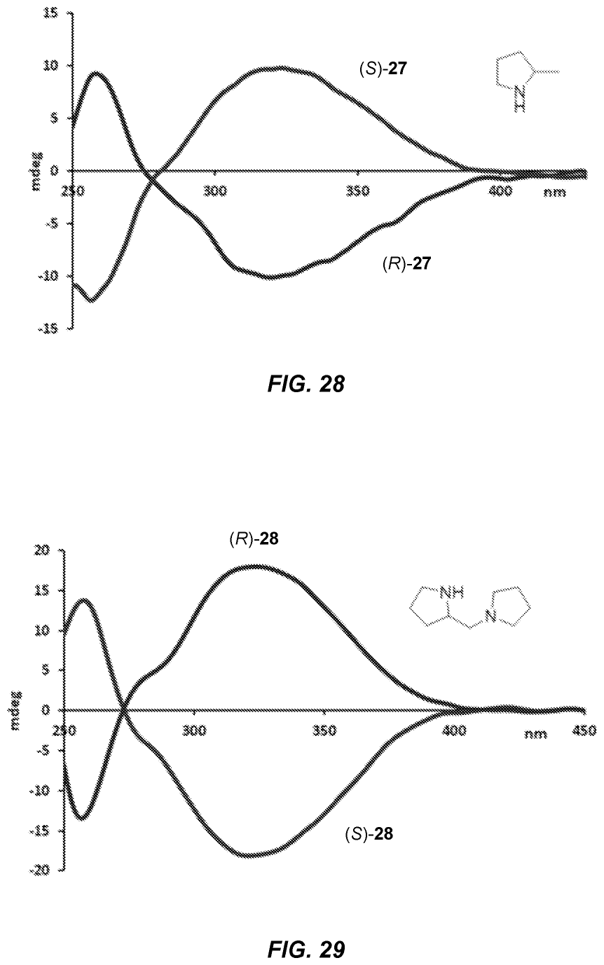

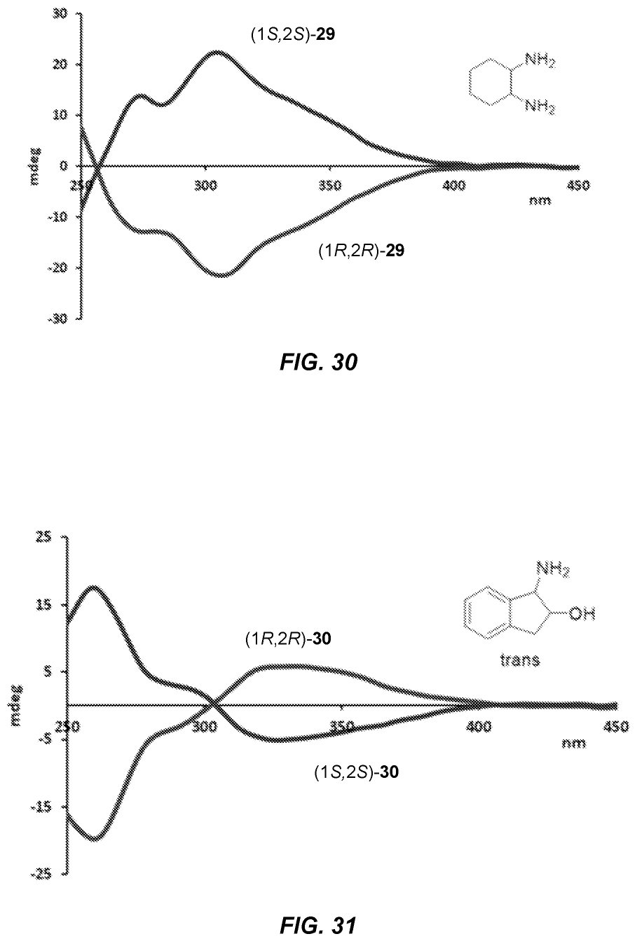

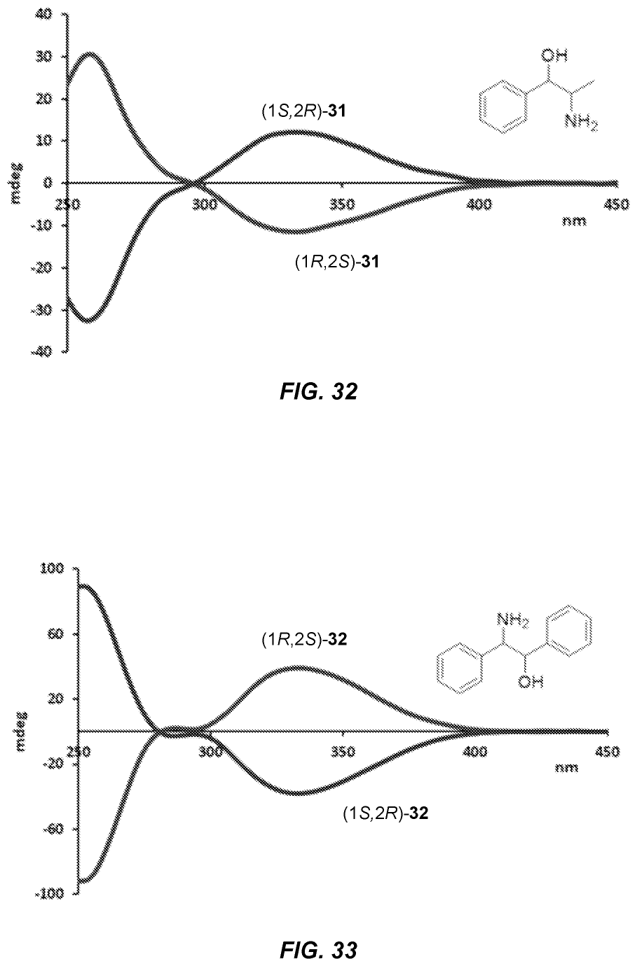

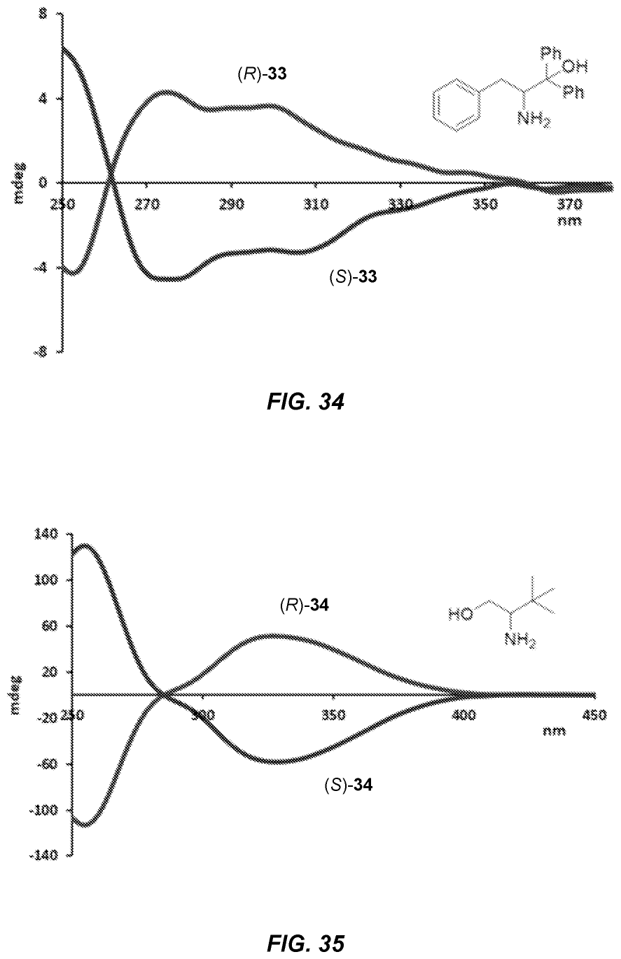

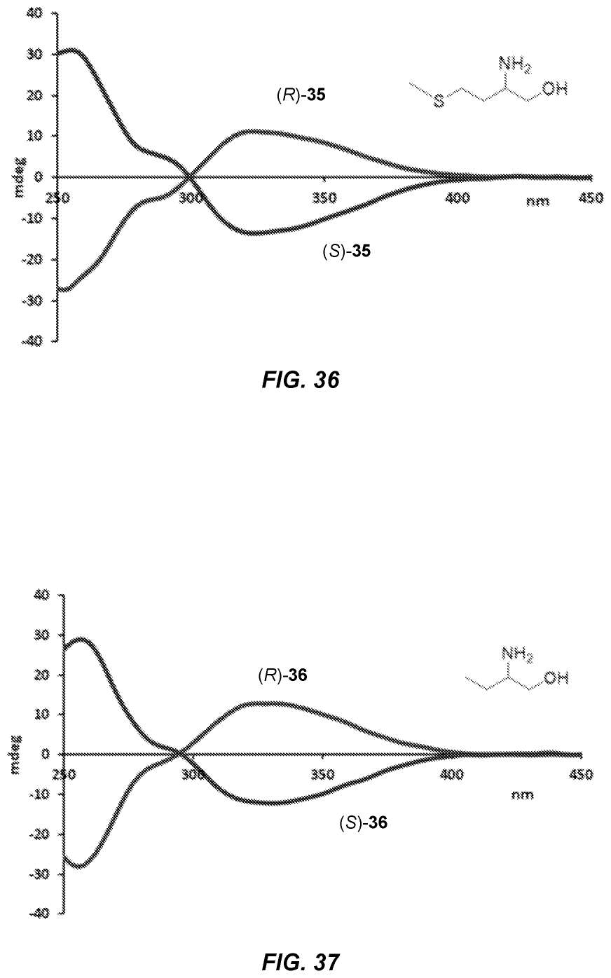

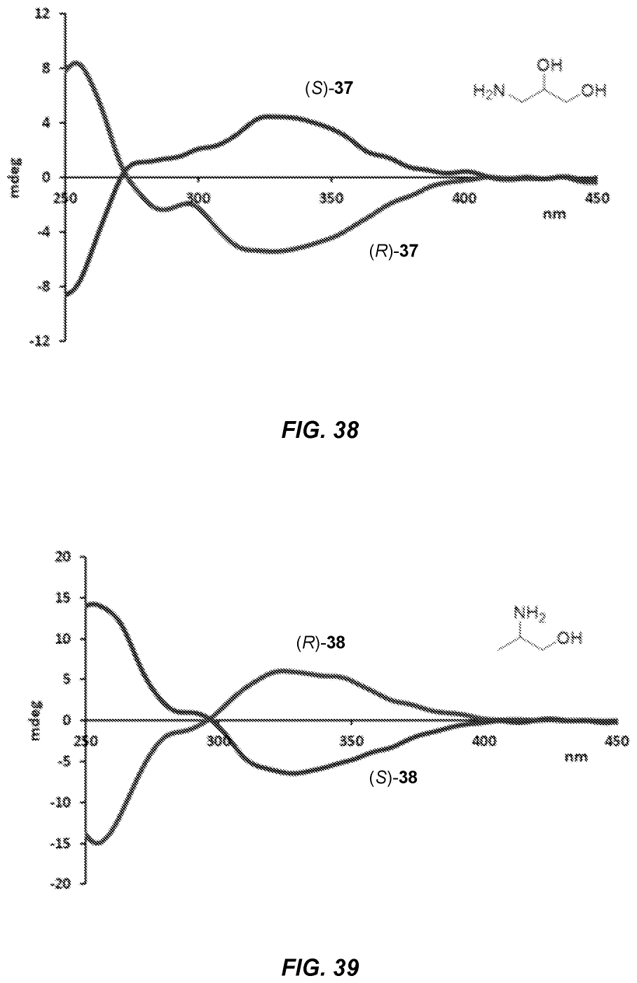

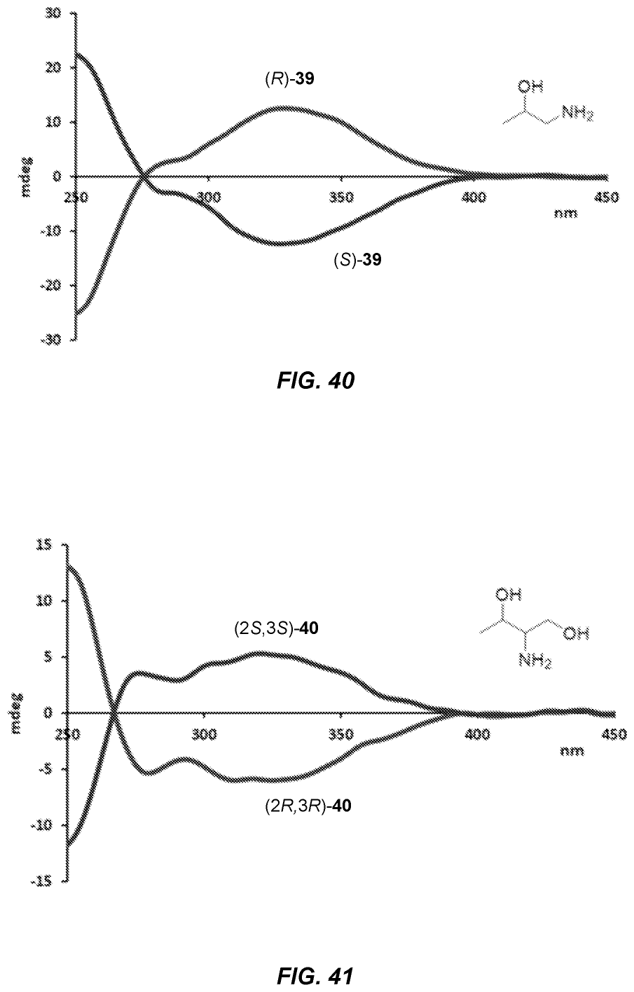

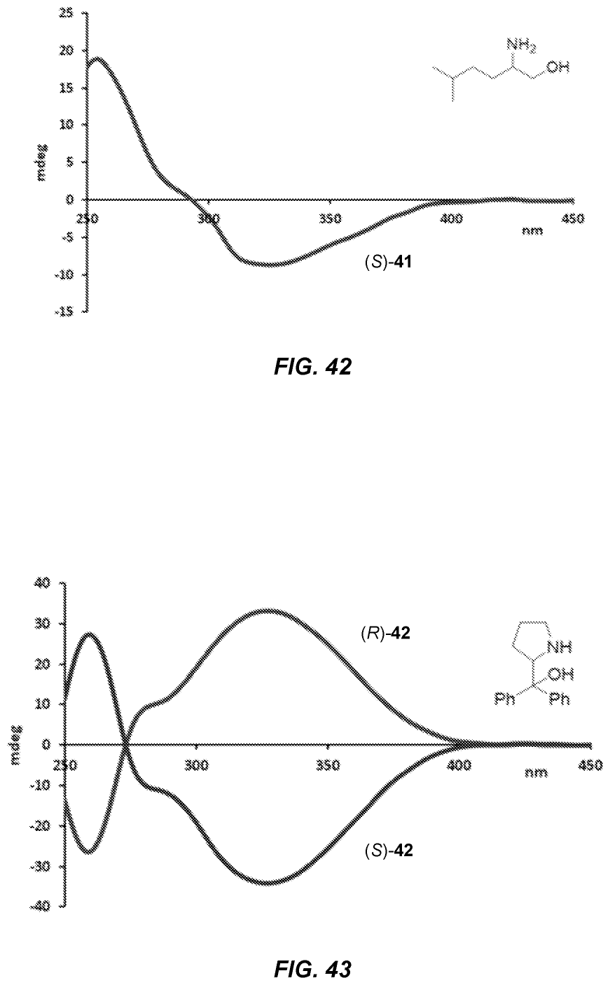

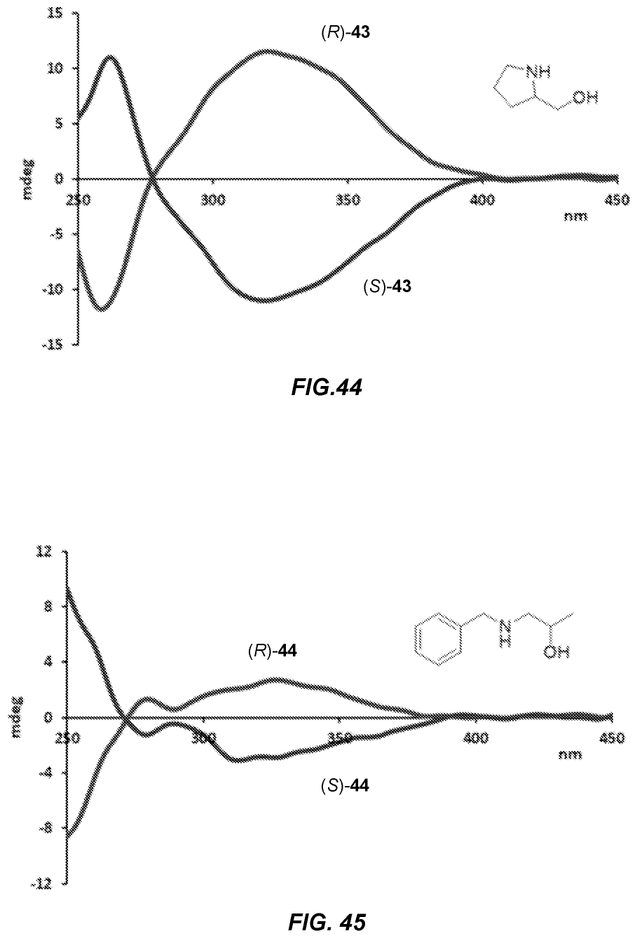

A solution of probe 1 (50.0 mM in ACN, 480.0 μL), chiral amines or amino alcohols 11-43 (50.0 mM in ACN, 400.0 μL), and Et₃N (1.2 equivalents) was stirred for 30 minutes. CD analysis was performed after dilution with ACN to the final concentration indicated below (FIGS. 12-45)(Scheme 2). The CD spectra were collected with a standard sensitivity of 100.0 mdeg, a data pitch of 0.5 nm, a bandwidth of 1 nm, in a continuous scanning mode with a scanning speed of 50.00 nm/min and a response of 1 s, using a quartz cuvette (1 cm path length). The data were baseline corrected and smoothed using a binomial equation.

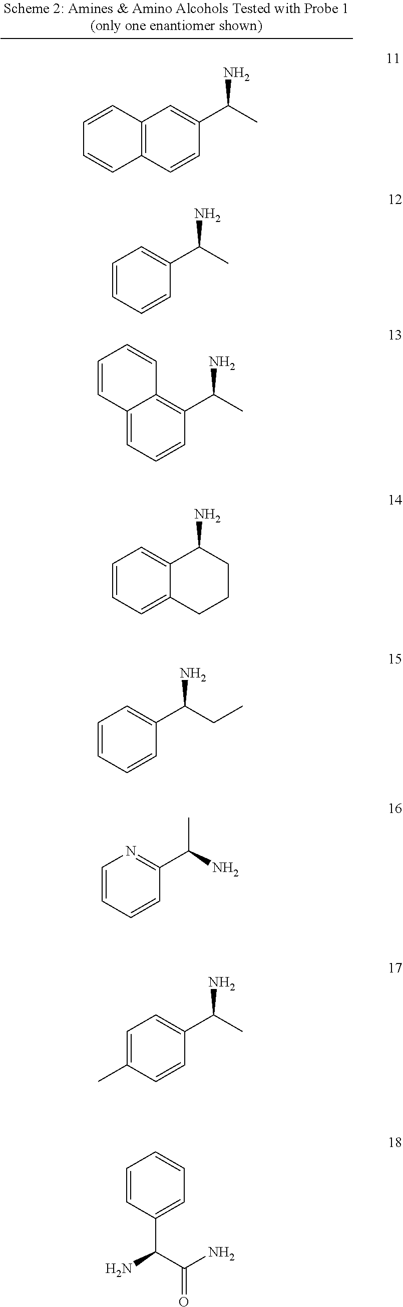

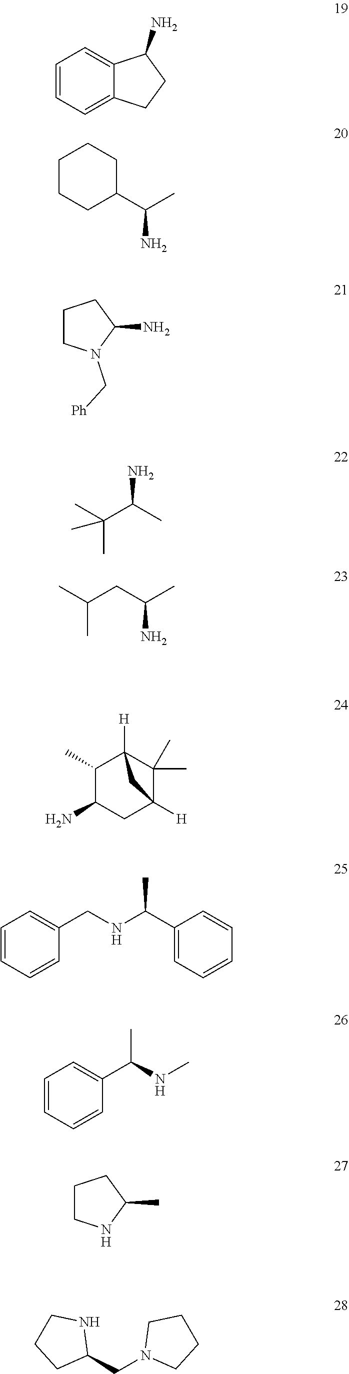

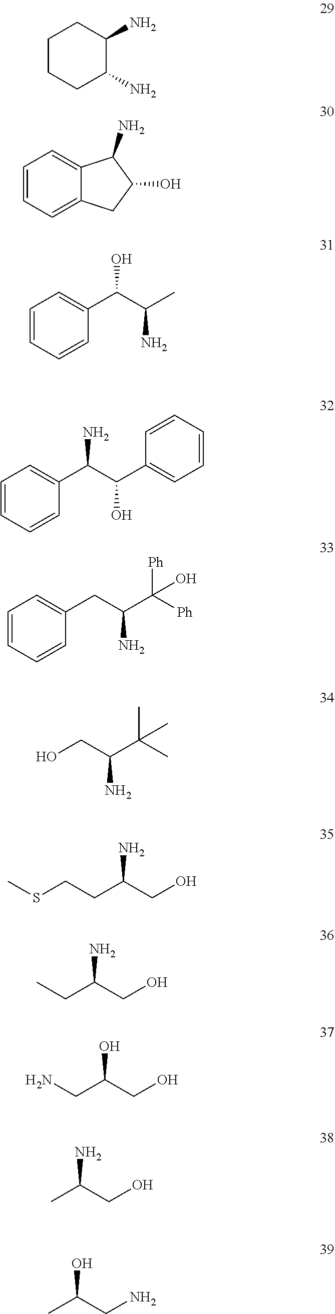

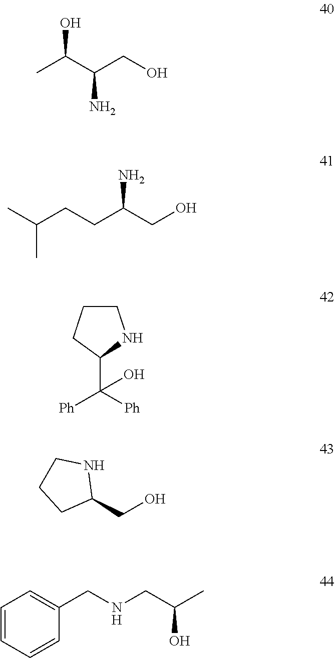

### Example 4—Sensing of Amino Acids by CD Analysis

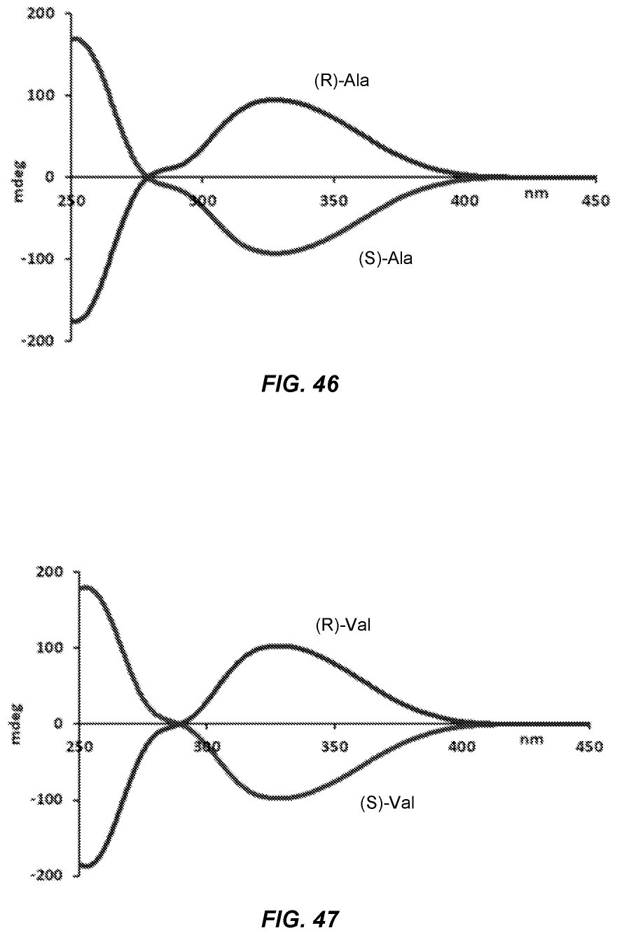

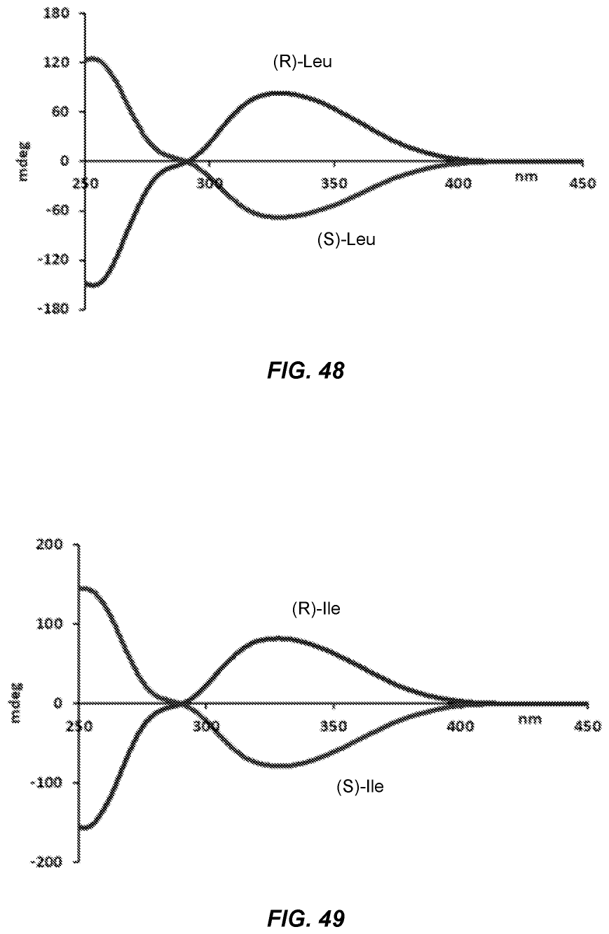

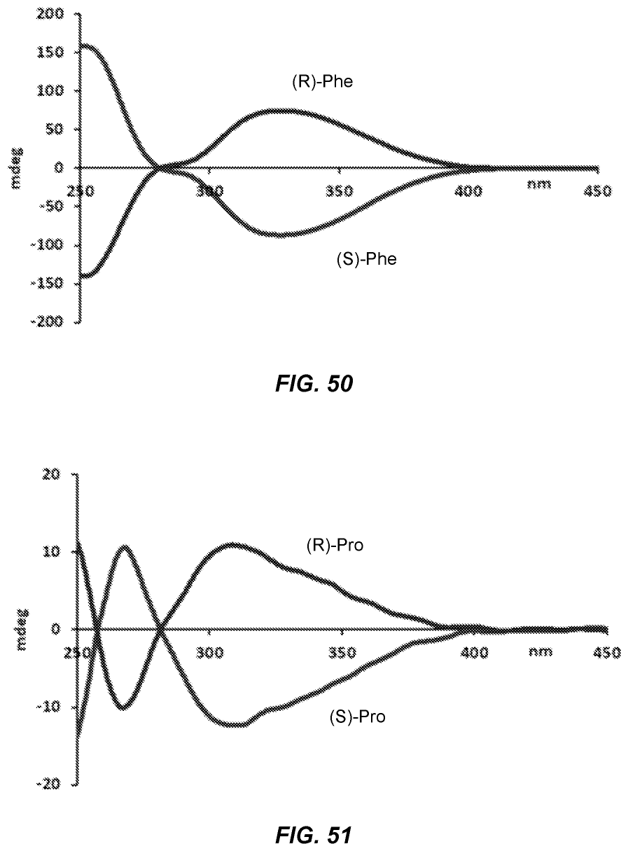

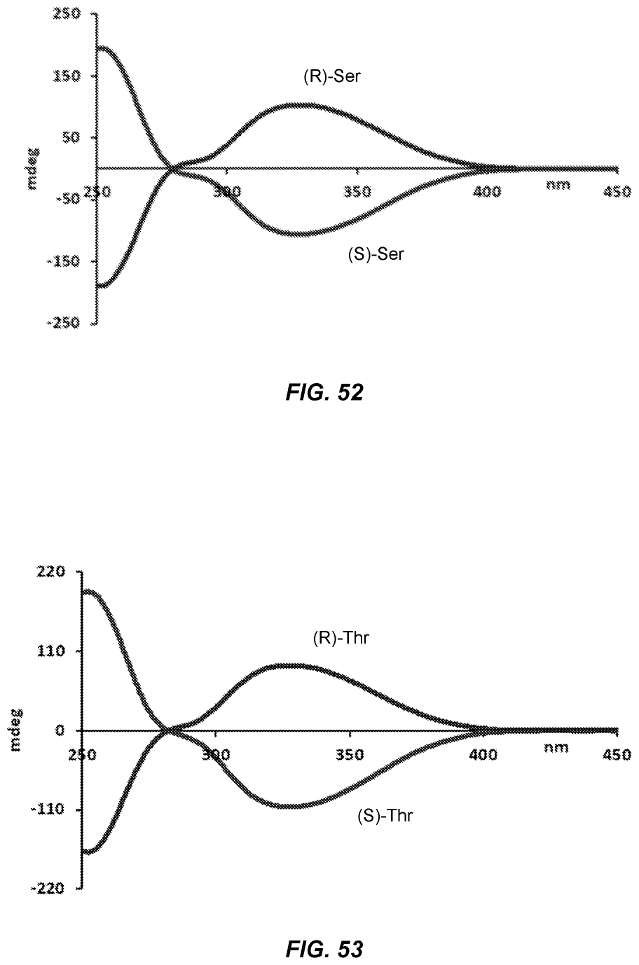

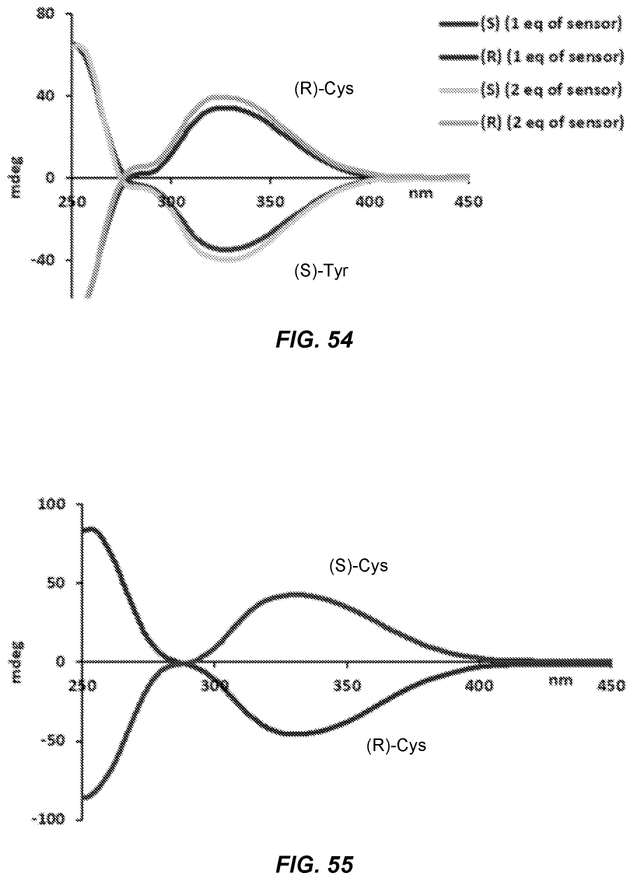

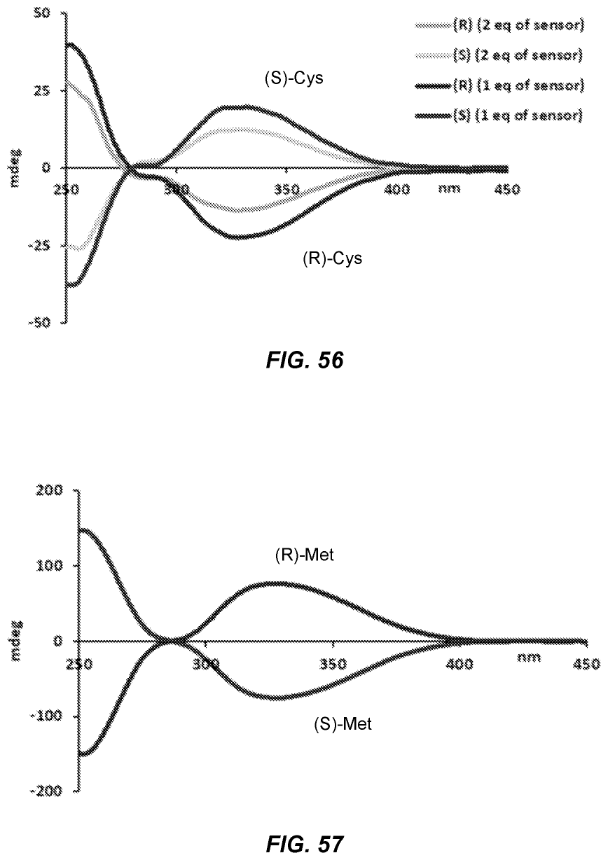

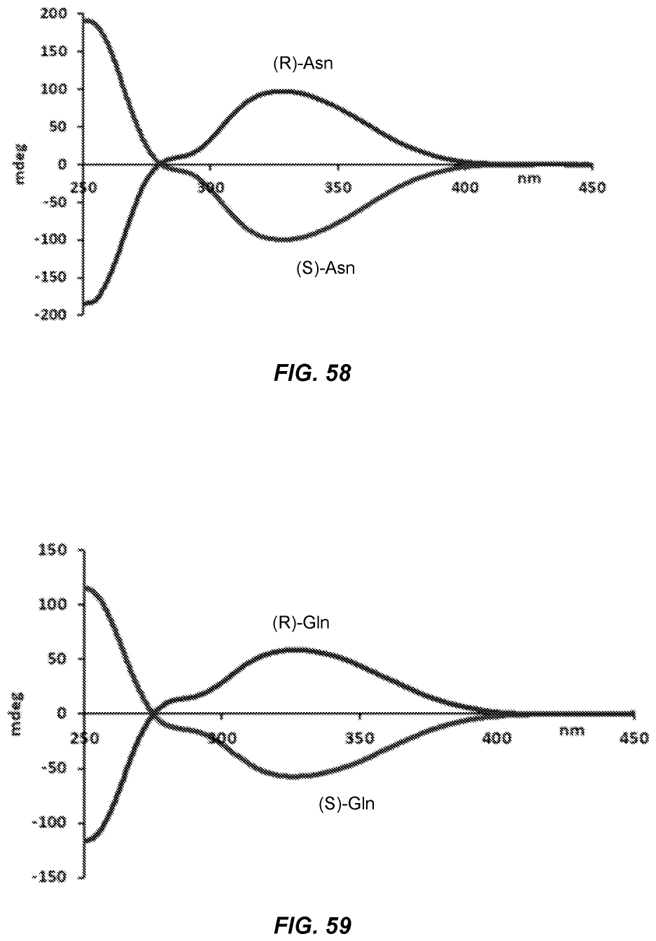

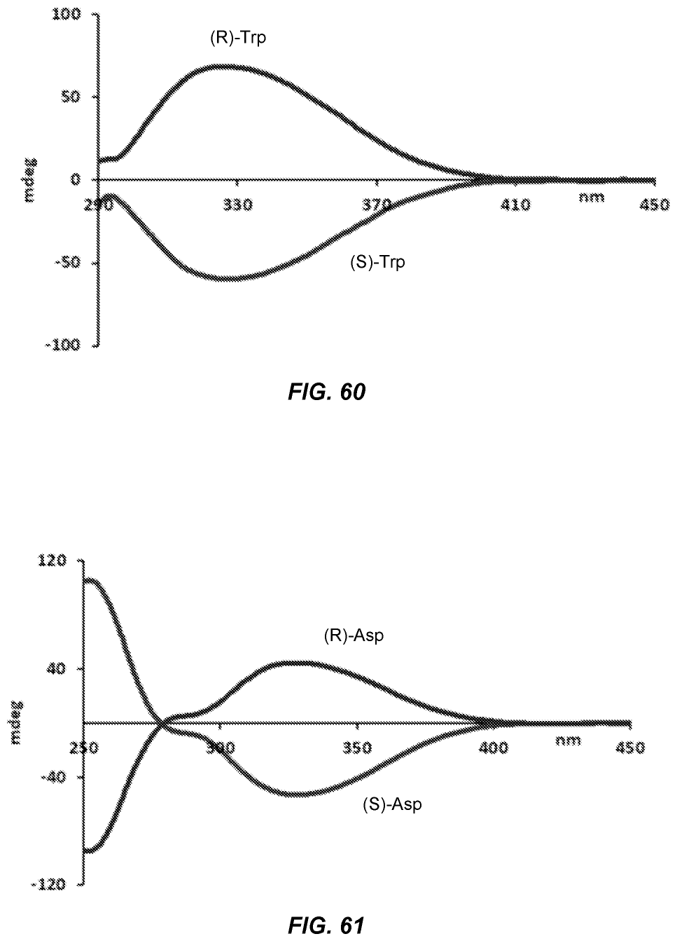

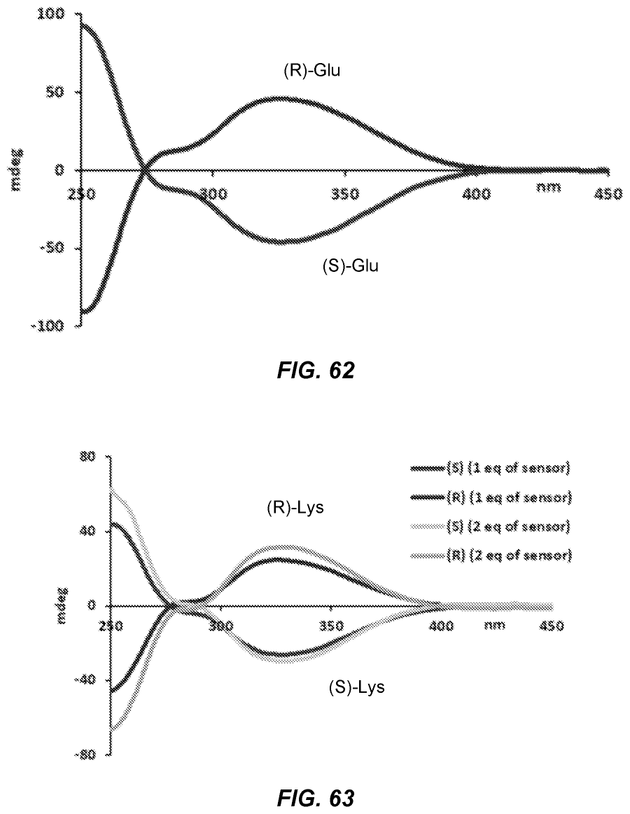

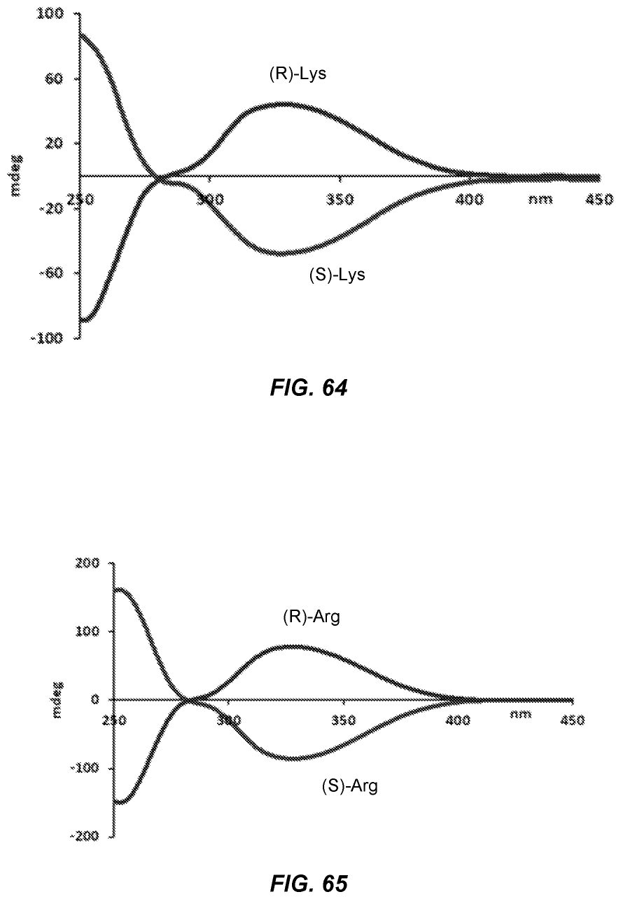

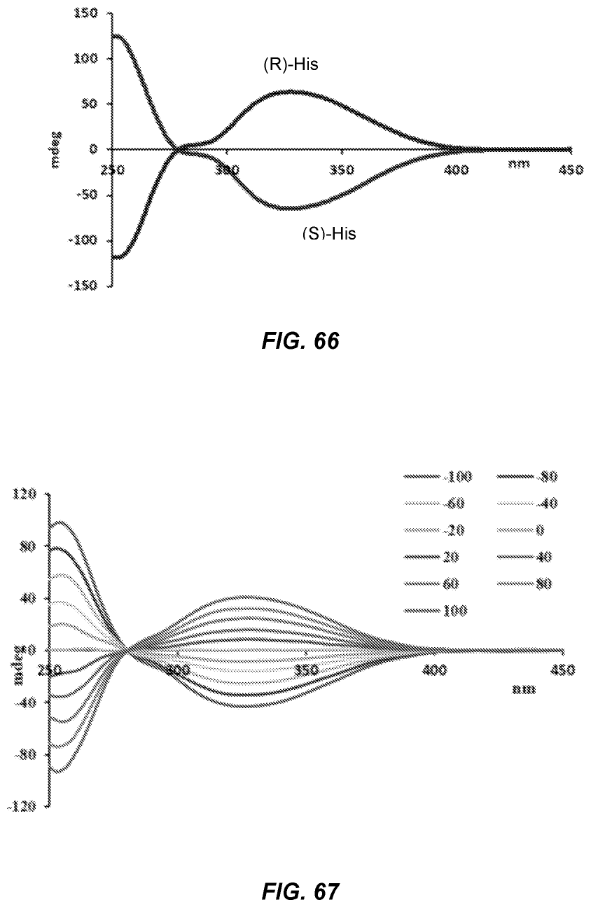

The utility of probe 1 was tested with all chiral standard amino acids (Scheme 3). Solutions of probe 1 (25.0 mM in acetonitrile, 480.0 μL) and an amino acid in pH 8.5 sodium borate buffer (25.0 mM in water, 400.0 μL) were combined and acetonitrile was used to dilute the total volume to 2.0 mL. The reaction was complete within 30 minutes. After 90 minutes CD measurements were taken by diluting 240.0 μL of the reaction mixture with 2.0 mL of acetonitrile unless otherwise noted (FIGS. 46-66). The CD spectra were collected with a standard sensitivity of 100 mdeg, a data pitch of 0.5 nm, a bandwidth of 1 nm, in a continuous scanning mode with a scanning speed of 500 nm/min and a response of 1 s, using a quartz cuvette (1 cm path length). The data were baseline corrected and smoothed using a binomial equation. The sodium borate buffer (0.25 M) was prepared using boric acid and sodium hydroxide in distilled water. The pH was adjusted to 8.5 using 5.0 M NaOH.

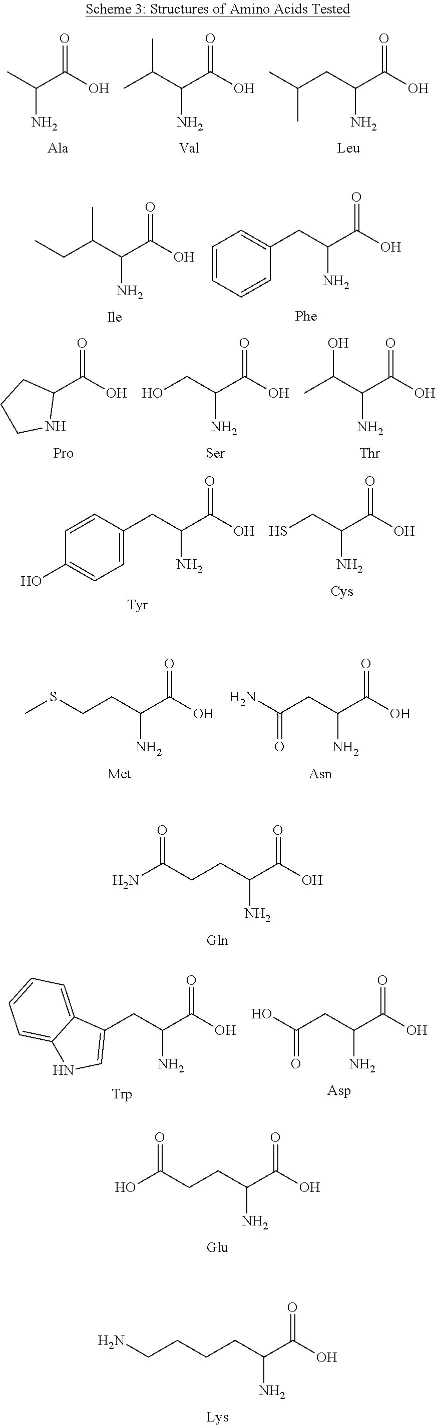

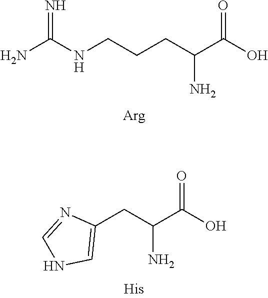 [SMILES: N=C(N)NCCCC(N)C(=O)O ; NC(Cc1c[nH]cn1)C(=O)O]

### Example 5—Quantitative Enantiomeric Ratio (Er) and Concentration Analysis

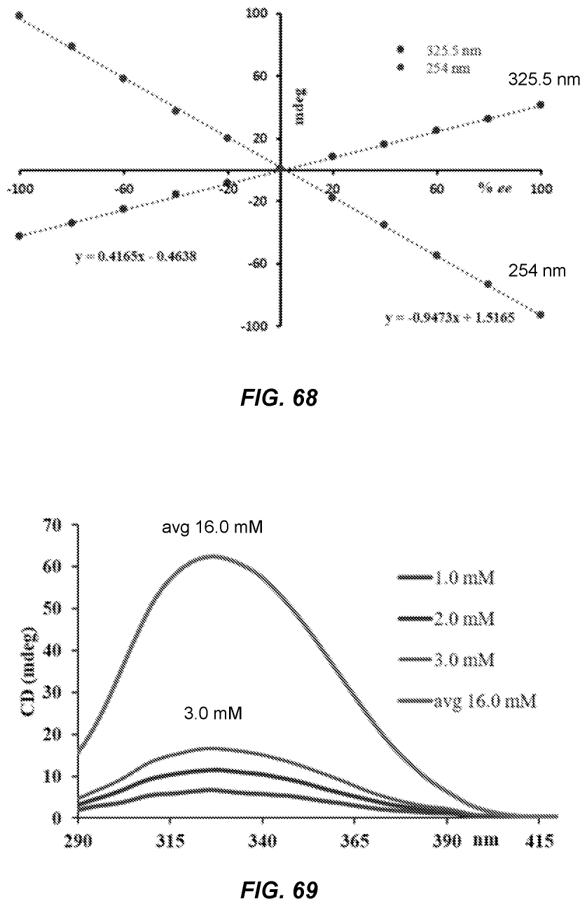

A calibration curve was constructed using samples containing 3,3-dimethylbutan-2-amine with varying enantiomeric composition. Probe 1 (50.0 mM in acetonitrile, 480.0 μL) and 3,3-dimethylbutan-2-amine (50.0 mM in acetonitrile, 400.0 μL) with varying ee's (+100.0, +80.0, +60.0, +40.0, +20.0, 0, −20.0, −40.0, −60.0, −80.0, −100.0%) were combined and stirred for 30 minutes. CD analysis was carried out by diluting 40.0 μL of the reaction mixture with ACN (2.0 mL) (FIG. 67). The CD amplitudes at 325.5 nm and 254.0 nm were plotted against the enantiomeric excess of 3,3-dimethylbutan-2-amine showing a perfectly linear relationship (FIG. 68).

### Example 6—Simultaneous Concentration, Absolute Configuration, and er Analysis of Amines

A sample containing enantioenriched (R)-3,3-dimethylbutan-2-amine (92.0:8.0 er, 12.0 mM) was analyzed. First, a 0.1 M stock solution in ACN was prepared. To 120.0 μL of this solution were added varying volumes (10.0, 20.0, 30.0 and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIG. 69). For the sample containing excess of 1, the CD sensing was performed in duplicate.

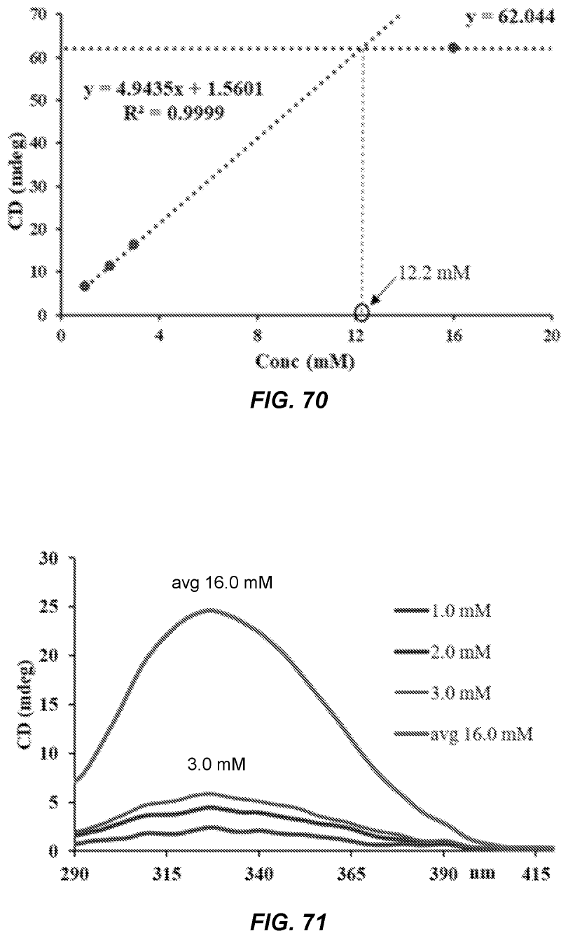

The experimentally obtained CD amplitudes at 324.0 nm were plotted against the concentrations of the sensor in the reaction mixture (FIG. 70). Linear regression analysis using the CD amplitudes obtained with 1 in the region of excess of the analyte showed a linear increase. A horizontal line parallel to the x-axis (slope=0) representing the range where the CD amplitude is stagnant because the sensor is in excess of the amine analyte was obtained. The x-value at the intersection of these two lines was used to determine the original concentration of the amine sample (keeping the sample dilution protocol described above in mind) as 12.2 mM. With the concentration of the analyte in hand, the enantiomeric composition was calculated by comparing the y-axis value (mdeg) with that of an enantiopure reference. This gave an enantiomeric ratio of 92.1:7.9. The absolute configuration was determined from the sign of the observed CD signal.

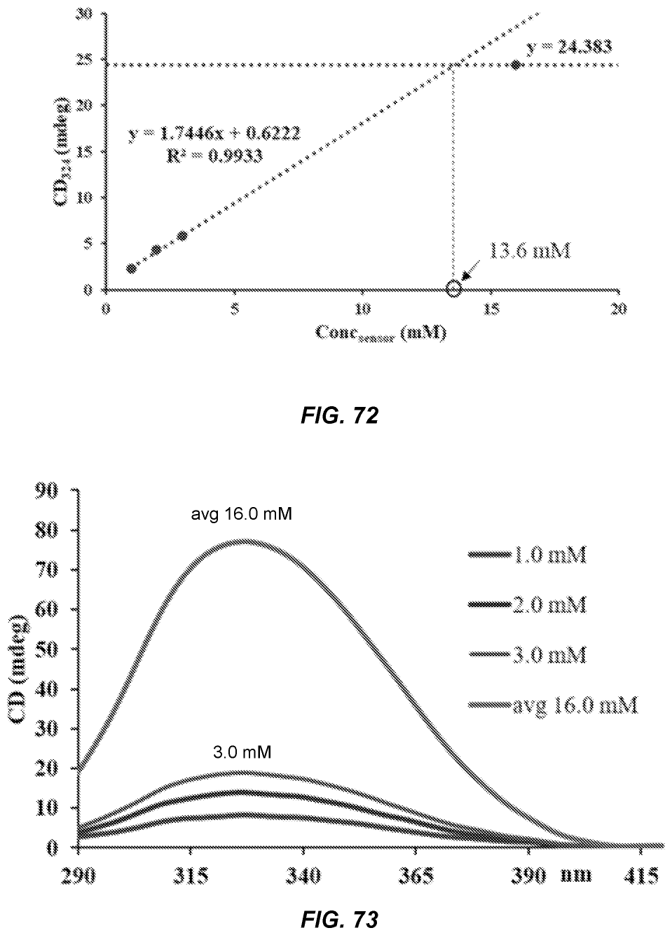

A sample containing enantioenriched (R)-3,3-dimethylbutan-2-amine (65.0:35.0 er, 14.0 mM) was analyzed. First, a 0.1 M stock solution in ACN was prepared. To 140.0 μL of this solution were added varying volumes (10.0, 20.0, 30.0, and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIGS. 71). For the sample containing excess of 1, the CD sensing was performed in duplicate. Then, the concentration and enantiomeric ratio were determined as 13.6 mM and 64.8:35.2 er using the protocol mentioned above (FIG. 72).

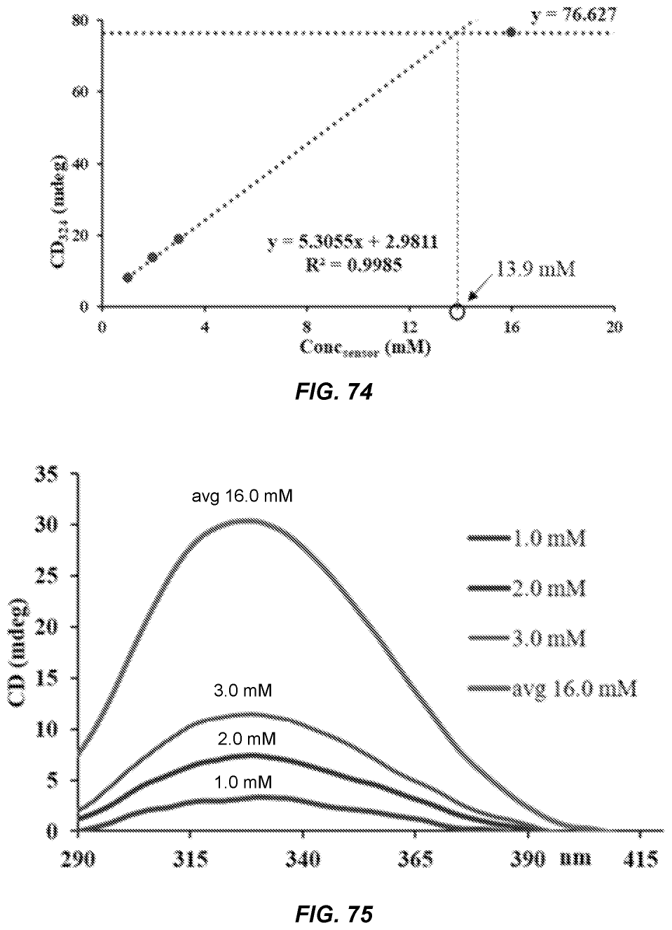

A sample containing enantioenriched (R)-3,3-dimethylbutan-2-amine (96.0:4.0 er, 14.0 mM) was analyzed. First, a 0.1 M stock solution in ACN was prepared. To 140.0 μL of this solution were added varying volumes (10.0, 20.0, 30.0, and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIG. 73). For the sample containing excess of 1, the CD sensing was performed in duplicate. Then, the concentration and enantiomeric ratio were determined as 13.9 mM and 95.8:4.2 er using the protocol mentioned above (FIG. 74).

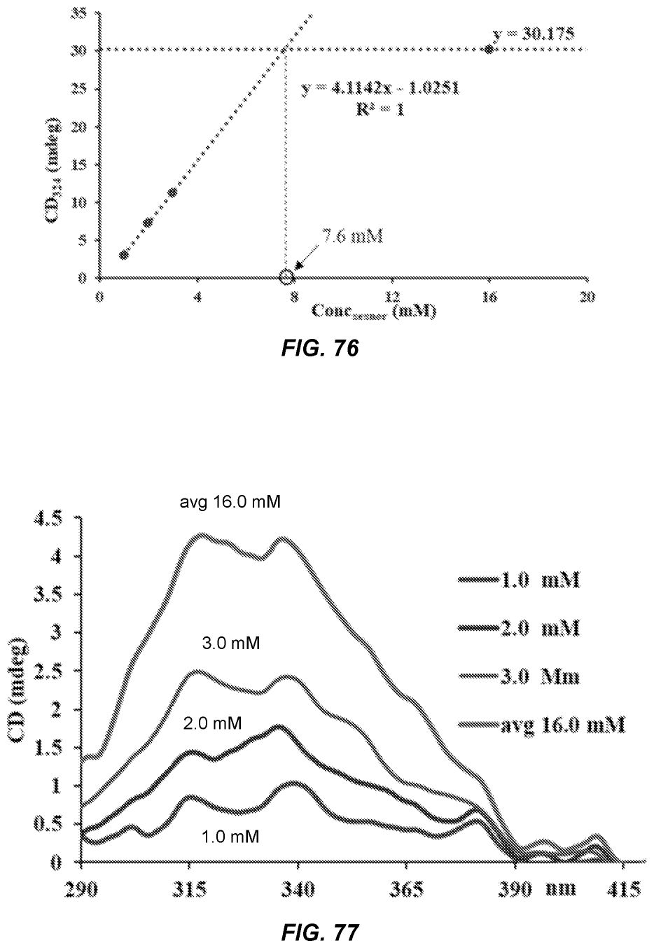

A sample containing enantioenriched (R)-3,3-dimethylbutan-2-amine (81.5:18.5 er, 8.0 mM) was analyzed. First, a 0.1 M stock solution in ACN was prepared. To 80.0 μL of this solution were added varying volumes (10.0, 20.0, 30.0, and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIG. 75). For the sample containing excess of 1, the CD sensing was performed in duplicate. Then, the concentration and enantiomeric ratio were determined as 7.6 mM and 81.9:18.1 er using the protocol mentioned above (FIG. 76).

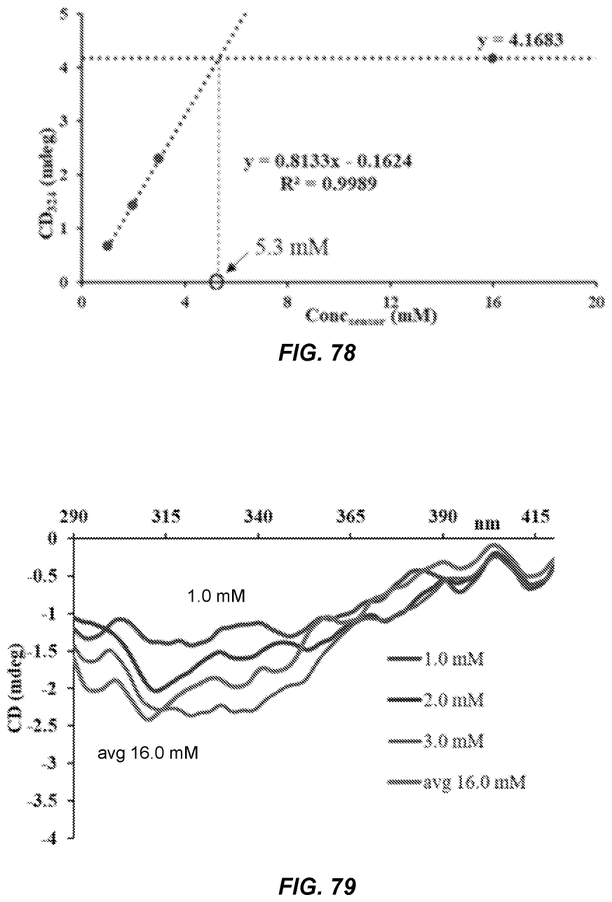

A sample containing enantioenriched (R)-3,3-dimethylbutan-2-amine (55.0:45.0 er, 6.0 mM) was analyzed. First, a 0.1 M stock solution in ACN was prepared. To 60.0 μL of this solution were added varying volumes (10.0, 20.0, 30.0, and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIG. 77). For the sample containing excess of 1, the CD sensing was performed in duplicate. Then, the concentration and enantiomeric ratio were determined as 5.3 mM and 56.7:43.3 er using the protocol mentioned above (FIG. 78).

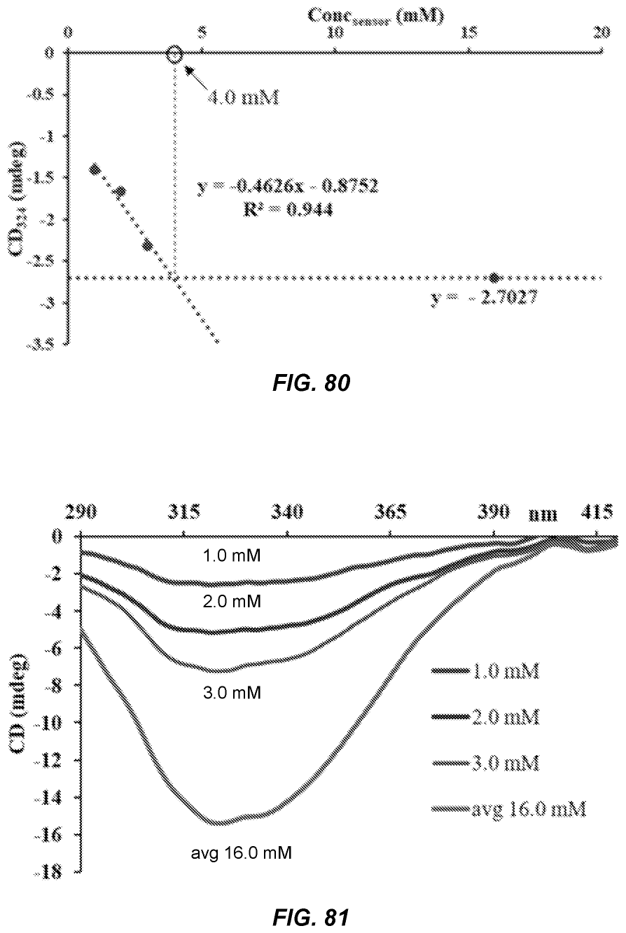

A sample containing enantioenriched (S)-3,3-dimethylbutan-2-amine (55.0:45.0 er, 5.0 mM) was analyzed. First, a 0.1 M stock solution in ACN was prepared. To 50.0 μL of this solution were added varying volumes (10.0, 20.0, 30.0, and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIG. 79). For the sample containing excess of 1, the CD sensing was performed in duplicate. Then, the concentration and enantiomeric ratio were determined as 4.0 mM and 55.5:45.5 er using the protocol mentioned above (FIG. 80).

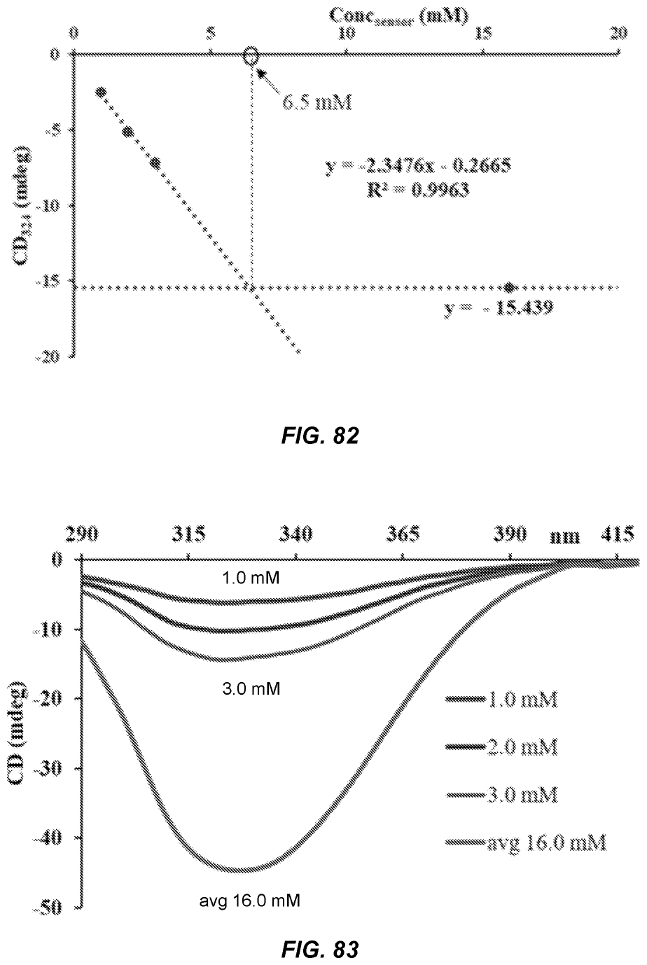

A sample containing enantioenriched (S)-3,3-dimethylbutan-2-amine (68.5:31.5 er, 7.0 mM) was analyzed. First, a 0.1 M stock solution in ACN was prepared. To 70.0 μL of this solution were added varying volumes (10.0, 20.0, 30.0, and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIG. 81). For the sample containing excess of 1, the CD sensing was performed in duplicate. Then, the concentration and enantiomeric ratio were determined as 6.5 mM and 69.2:30.9 er using the protocol mentioned above (FIG. 82).

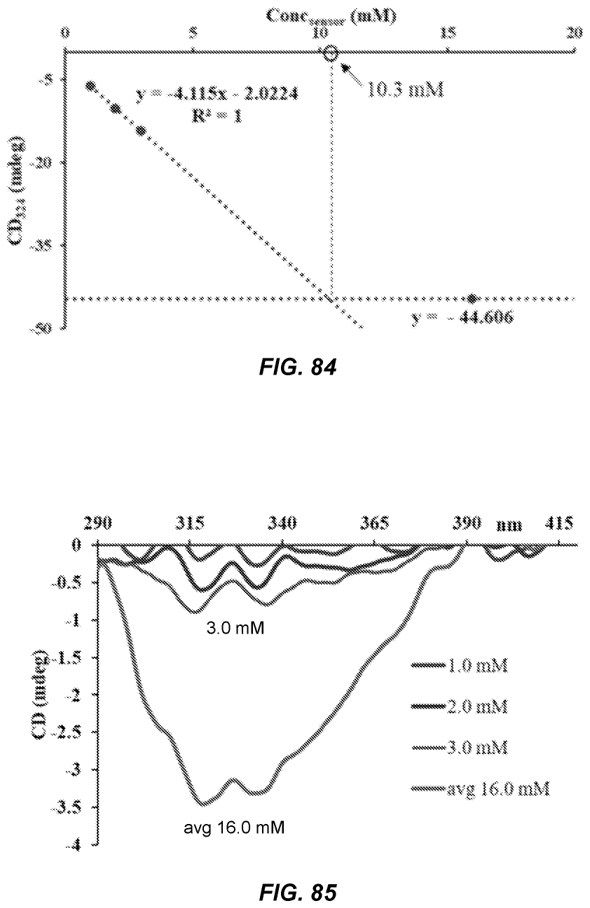

A sample containing enantioenriched (S)-3,3-dimethylbutan-2-amine (85.0:15.0 er, 10.0 mM) was analyzed. First, a 0.1 M stock solution in ACN was prepared. To 100.0 μL of this solution were added varying volumes (10.0, 20.0, 30.0, and 160.0 μL) of probe 1 (0.1 M in acetonitrile) and 1.2 equivalents of triethylamine. The total reaction volume was adjusted to 1.0 mL using ACN and stirred for 30 minutes. CD analysis was performed after diluting a 210.0 μL aliquot with 2.0 mL of CAN (FIG. 83). For the sample containing excess of 1, the CD sensing was performed in duplicate. Then, the concentration and enantiomeric ratio were determined as 10.3 mM and 64.8:35.2 er using the protocol mentioned above (FIG. 84).

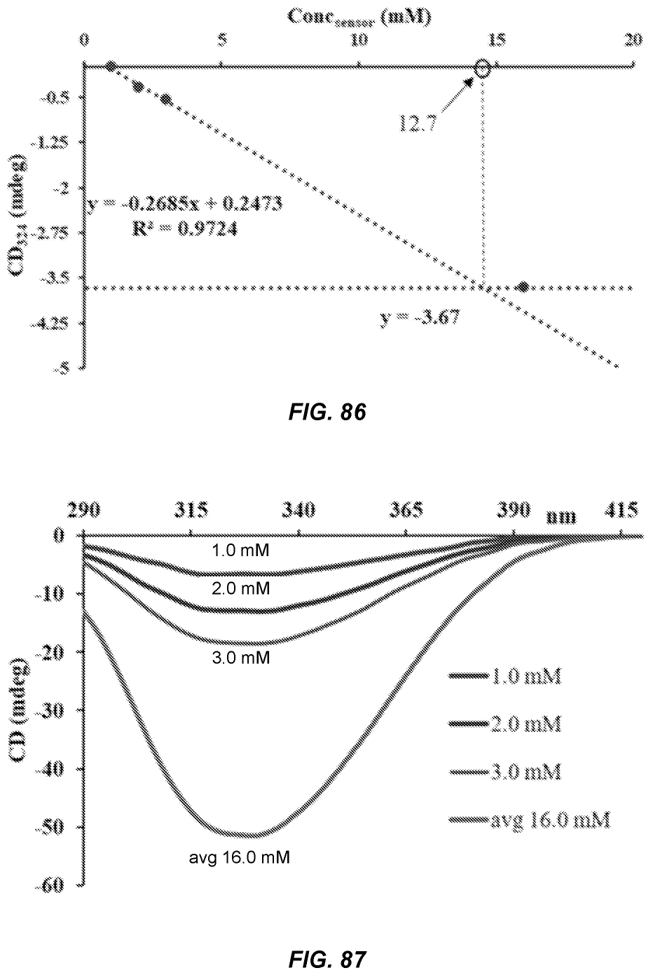

A sample containing enantioenriched (S)-3,3-dimethylbutan-2-amine (52.5:47.5 er, 13.0 mM) was analyzed. First, a 0.1 M stock solution in ACN was prepared. To 130.0 μL of this solution were added varying volumes (10.0, 20.0, 30.0, and 160.0 μL) of probe 1 (0.1 M in acetonitrile) and 1.2 equiv. of triethylamine. The reaction mixture containing 13.0 mM of the amine was diluted to a total volume of 1.0 mL using ACN and stirred for 30 minutes. CD analysis was performed after diluting a 210.0 μL aliquot with 2.0 mL of CAN (FIG. 85). For the sample containing excess of 1, the CD sensing was performed in duplicate. Then, the concentration and enantiomeric ratio were determined as 12.7 mM and 52.4:47.6 er using the protocol mentioned above (FIG. 86).

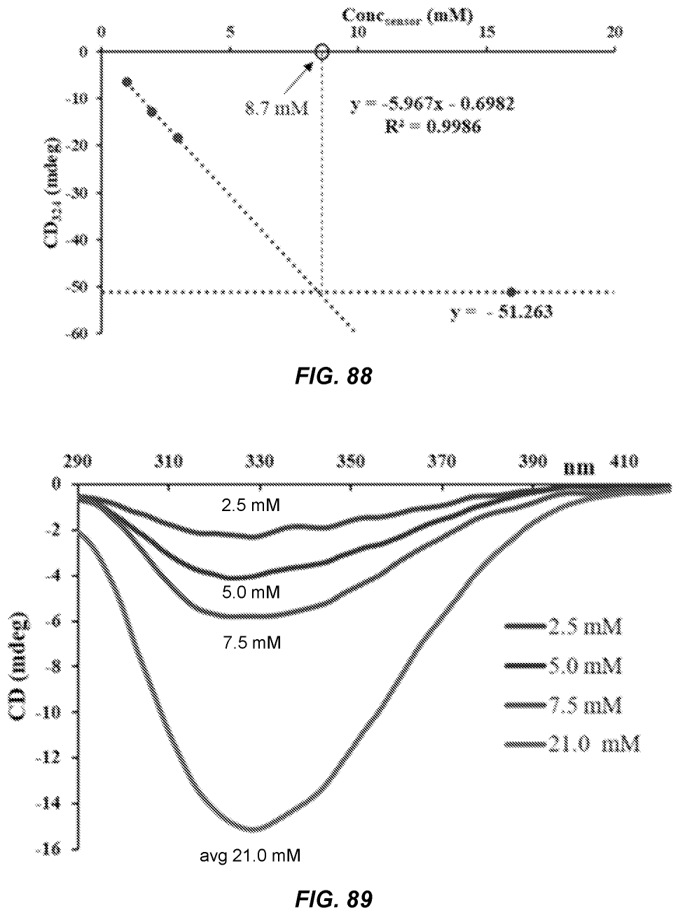

A sample containing enantioenriched (S)-3,3-dimethylbutan-2-amine (99.0:1.0 er, 9.0 mM) was analyzed. First, a 0.1 M stock solution in ACN was prepared. To 90.0 μL of this solution were added varying volumes (10.0, 20.0, 30.0, and 160.0 μL) of probe 1 (0.1 M in acetonitrile) and 1.2 equiv. of triethylamine. The reaction mixture containing 9.0 mM of the amine was diluted to a total volume of 1.0 mL using ACN and stirred for 30 minutes. CD analysis was performed after diluting a 210.0 μL aliquot with 2.0 mL of CAN (FIG. 87). For the sample containing excess of 1, the CD sensing was performed in duplicate. Then, the concentration and enantiomeric ratio were determined as 8.7 mM and 98.8:1.2 er using the protocol mentioned above (FIG. 88).

TABLE 2
Analysis of Absolute Configuration, er, and Concentration of
3,3-Dimethylbutylamine Samples with Probe 1 Using CD Sensing

| | |
|---|---|
| Sample composition | CD Sensing results |

| Abs | Conc | | Abs | Conc | |
|---|---|---|---|---|---|
| config. | (mM) | er | config. | (mM) | er |
| S | 5.0 | 55.0:45.0 | S | 4.0 | 55.5:45.5 |
| S | 7.0 | 68.5:31.5 | S | 6.5 | 69.2:30.9 |
| S | 10.0 | 85.0:15.0 | S | 10.3 | 84.6:15.4 |
| S | 13.0 | 52.5:47.5 | S | 12.7 | 52.4:47.6 |
| S | 9.0 | 99.0:1.0 | S | 8.7 | 98.8:1.2 |
| R | 6.0 | 54.0:46.0 | R | 5.3 | 54.2:45.8 |
| R | 8.0 | 81.5:18.5 | R | 7.6 | 81.9:18.1 |
| R | 12.0 | 92.0:8.0 | R | 12.2 | 92.1:7.9 |
| R | 14.0 | 65.0:35.0 | R | 13.6 | 64.8:35.2 |
| R | 14.0 | 96.0:4.0 | R | 13.9 | 95.8:4.2 |

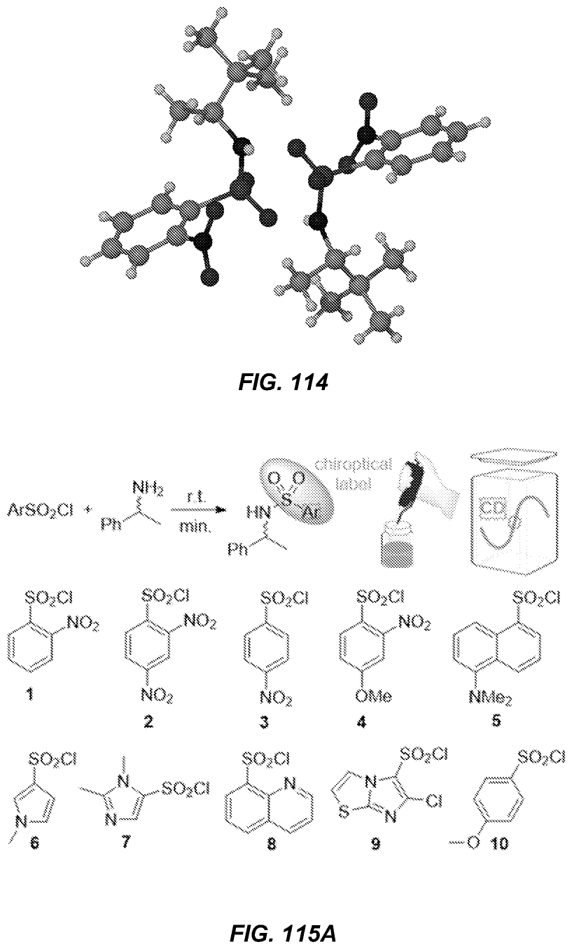

A single crystal of (S)—N-(3,3-dimethylbutan-2-yl)-2-nitrobenzenesulfonamide was obtained by slow evaporation of a solution in ethyl hexanes:ethylacetate:chloroform (1:1:1). Single crystal X-ray analysis was performed at 100 K using a Siemens platform diffractometer with graphite monochromated Cu-Kα radiation (λ=1.54178 Å) (FIG. 114). Data were integrated and corrected using the APEX 3 program. The structures were solved by direct methods and refined with full-matrix least-square analysis using SHELX-97-2 software. Non-hydrogen atoms were refined with anisotropic displacement parameter. Crystal data: C₁₂H₁₈N₂O₄S, M=286.35, colorless prism, 0.456×0.384×0.192 mm³, monoclinic, space group P2₁, a=8.8033(5) Å, b=14.5225(7) Å, c=11.0887(6) Å, V=1403.83(13) Å³, Z=4.

### Example 7—Simultaneous Concentration, Absolute Configuration, and Er Analysis of Amino Acids

A sample containing enantioenriched L-Ala (55:45 er, 20.0 mM) was analyzed. First, a 0.1 M stock solution in water was prepared. To 200.0 μL of this solution were added varying volumes (25.0, 50.0, 75.0, and 210.0 μL) of probe 1 (0.1 M in acetonitrile) and 2.0 equiv. of K₂CO₃. The reaction mixture containing 20.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 89). For the sample with excess of 1, the CD sensing was performed in duplicate.

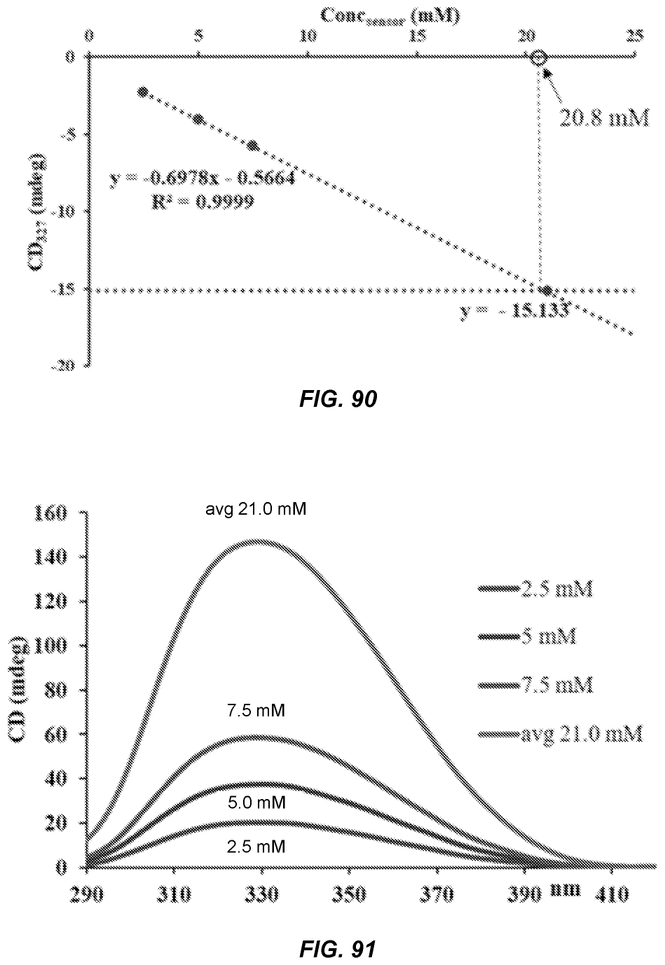

The experimentally obtained CD amplitudes at 327.0 nm were plotted against the concentrations of the sensor in the reaction mixture (FIG. 90). Linear regression analysis using the CD amplitudes obtained with 1 in the region of excess of the analyte showed a linear increase. A horizontal line parallel to the x-axis (slope=0) representing the range where the CD amplitude is stagnant because the sensor is in excess of the amine analyte was obtained. The x-value at the intersection of these two lines was used to determine the original concentration of the amine sample (keeping the sample dilution protocol described above in mind) as 20.8 mM. With the concentration of the analyte in hand, the enantiomeric composition was calculated by comparing the y-axis value (mdeg) to that of an enantiopure reference. This gave an enantiomeric ratio of 54.9:45.1.

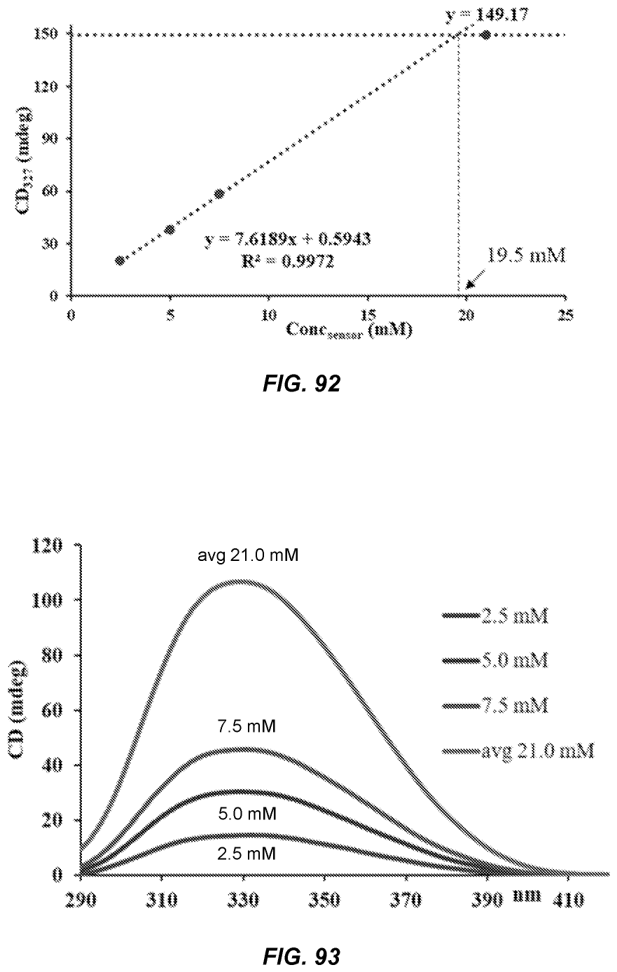

A sample containing enantioenriched D-Ala (97.5:2.5 er, 19.0 mM) was analyzed. First, a 0.1 M stock solution in water was prepared. To 190.0 μL of this solution were added varying volumes (25.0, 50.0, 75.0, and 210.0 μL) of probe 1 (0.1 M in acetonitrile) and 2.0 equiv. of K₂CO₃. The reaction mixture containing 19.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 91). For the sample with excess of 1, the CD sensing was performed in duplicate. Then, the concentration and enantiomeric ratio were determined as 19.5 mM and 97.4:2.6 er using the protocol mentioned above (FIG. 92).

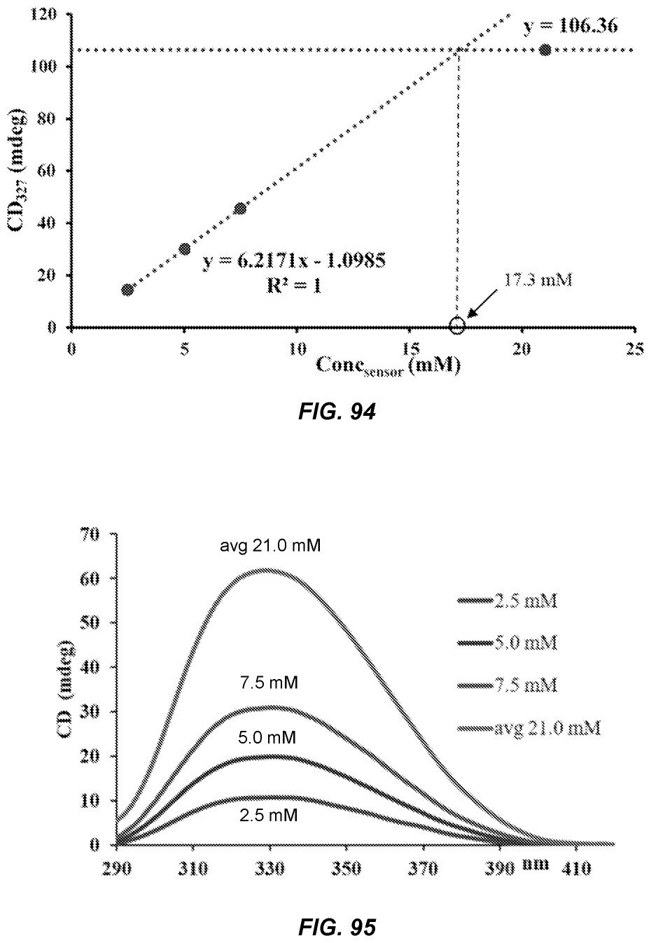

A sample containing enantioenriched D-Ala (88.0:12.0 er, 17.0 mM) was analyzed. First, a 0.1 M stock solution in water was prepared. To 170.0 μL of this solution were added varying volumes (25.0, 50.0, 75.0, and 210.0 μL) of probe 1 (0.1 M in acetonitrile) and 2.0 equiv. of K₂CO₃. The reaction mixture containing 17.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 93). For the sample with excess of 1, the CD sensing was performed in duplicate. Then, the concentration and enantiomeric ratio were determined as 17.3 mM and 88.1:11.9 er using the protocol mentioned above (FIG. 94).

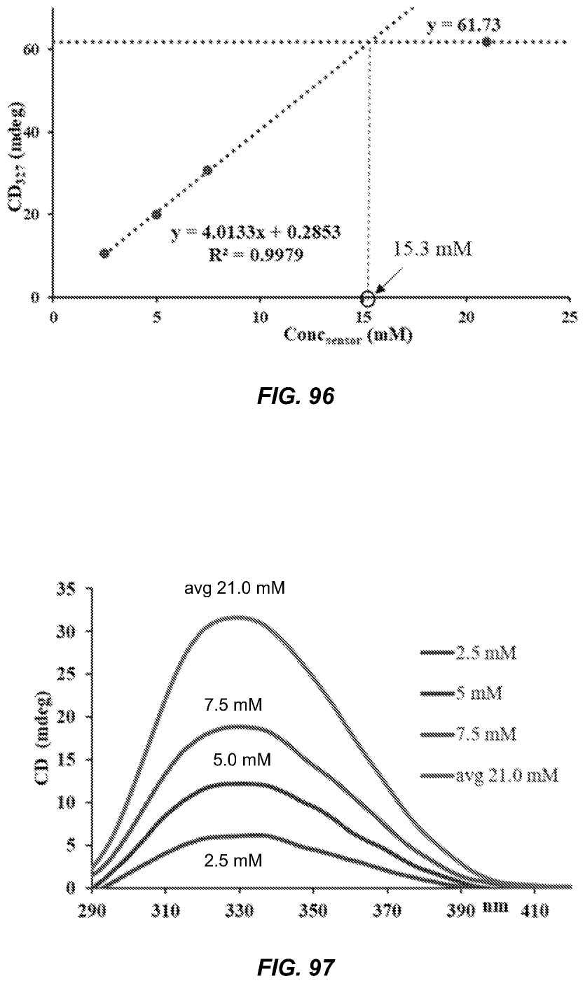

A sample containing enantioenriched D-Ala (76.0:24.0 er, 15.0 mM) was analyzed. First, a 0.1 M stock solution in water was prepared. To 150.0 μL of this solution were added varying volumes (25.0, 50.0, 75.0, and 210.0 μL) of probe 1 (0.1 M in acetonitrile) and 2.0 equiv. of K₂CO₃. The reaction mixture containing 15.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 95). For the sample with excess of 1, the CD sensing was performed in duplicate. Then, the concentration and enantiomeric ratio were determined as 15.3 mM and 75.0:25.0 er using the protocol mentioned above (FIG. 96).

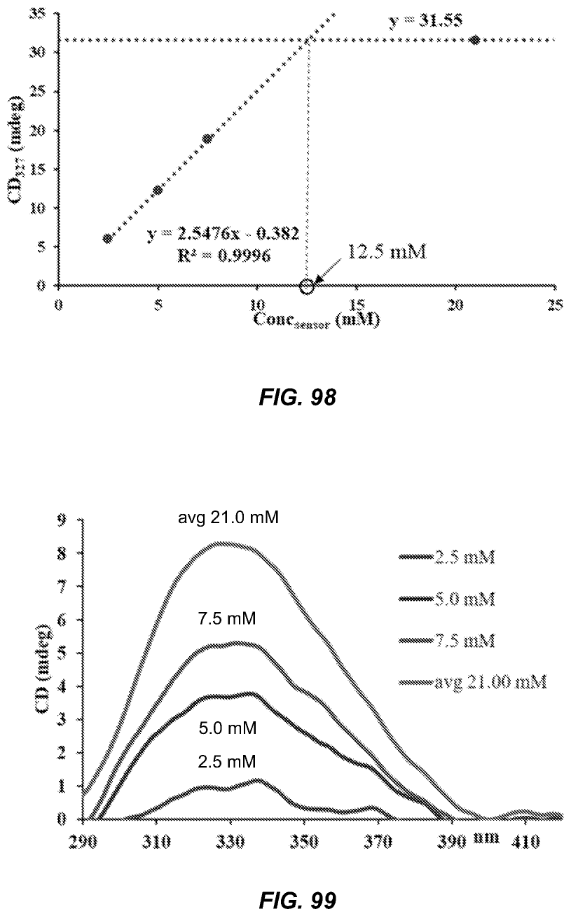

A sample containing enantioenriched D-Ala (66.0:34.0 er, 13.0 mM) was analyzed. First, a 0.1 M stock solution in water was prepared. To 130.0 μL of this solution were added varying volumes (25.0, 50.0, 75.0, and 210.0 μL) of probe 1 (0.1 M in acetonitrile) and 2.0 equiv. of K₂CO₃. The reaction mixture containing 13.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 97). For the sample with excess of 1, the CD sensing was performed in duplicate. Then, the concentration and enantiomeric ratio were determined as 12.5 mM and 65.8:34.2 er using the protocol mentioned above (FIG. 98).

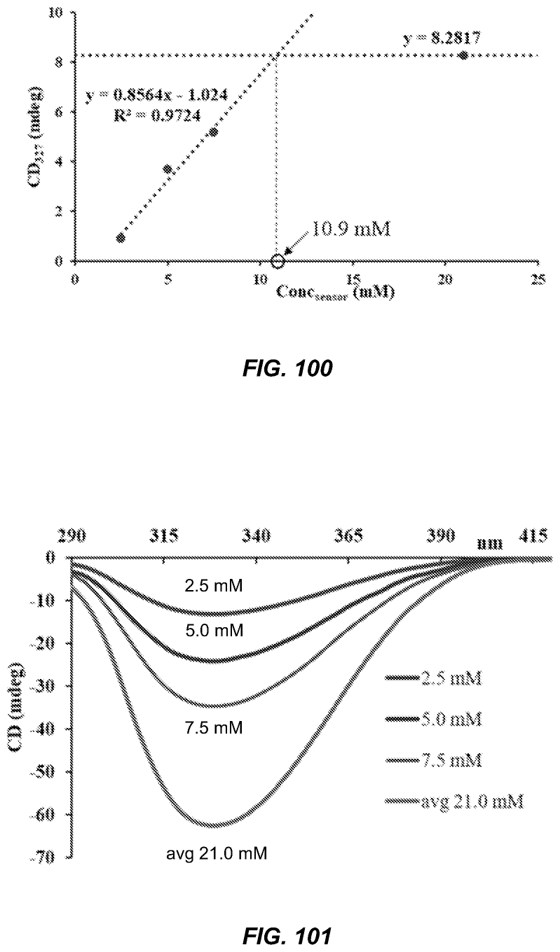

A sample containing enantioenriched D-Ala (55.0:45.0 er, 11.0 mM) was analyzed. First, a 0.1 M stock solution in water was prepared. To 110.0 μL of this solution were added varying volumes (25.0, 50.0, 75.0, and 210.0 μL) of probe 1 (0.1 M in acetonitrile) and 2.0 equiv. of K₂CO₃. The reaction mixture containing 11.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 99). For the sample with excess of 1, the CD sensing was performed in duplicate. Then, the concentration and enantiomeric ratio were determined as 10.9 mM and 54.7:45.3 er using the protocol mentioned above (FIG. 100).

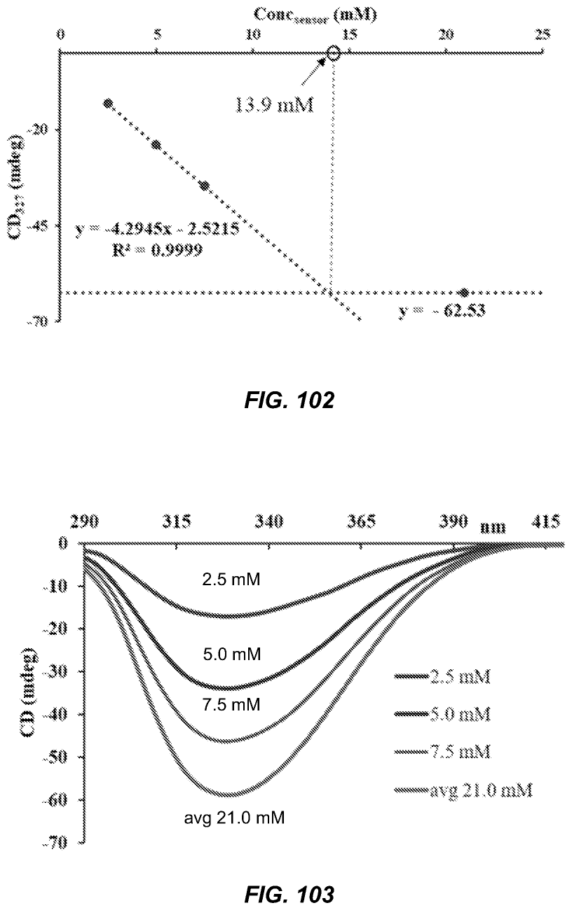

A sample containing enantioenriched L-Ala (80.5:19.5 er, 14.0 mM) was analyzed. First, a 0.1 M stock solution in water was prepared. To 140.0 μL of this solution were added varying volumes (25.0, 50.0, 75.0, and 210.0 μL) of probe 1 (0.1 M in acetonitrile) and 2.0 equiv. of K₂CO₃. The reaction mixture containing 14.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 101). For the sample with excess of 1, the CD sensing was performed in duplicate. Then, the concentration and enantiomeric ratio were determined as 13.9 mM and 85.1:14.9 er using the protocol mentioned above (FIG. 102).

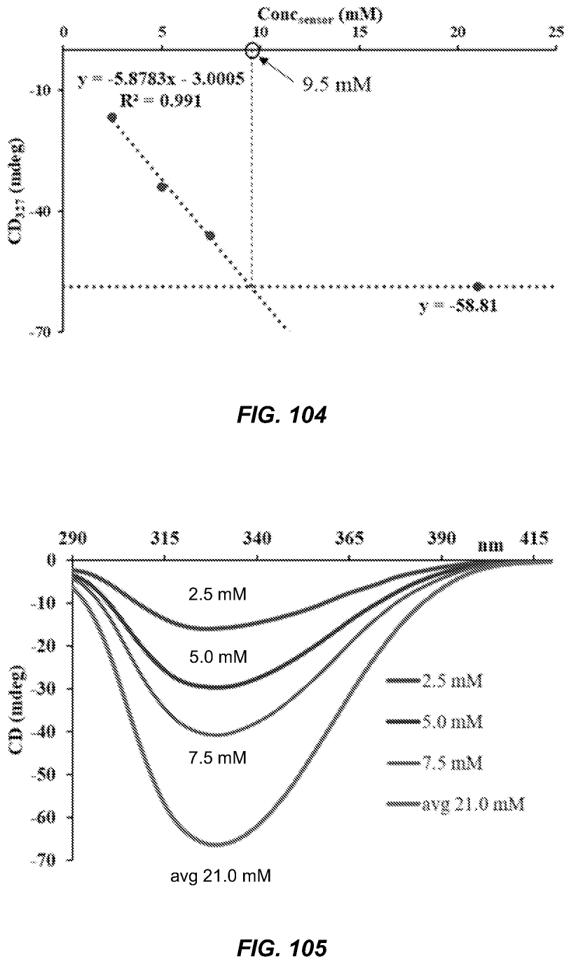

A sample containing enantioenriched L-Ala (91.0:9.0 er, 10.0 mM) was analyzed. First, a 0.1 M stock solution in water was prepared. To 100.0 μL of this solution were added 30 varying volumes (25.0, 50.0, 75.0, and 210.0 μL) of probe 1 (0.1 M in acetonitrile) and 2.0 equiv. of K₂CO₃. The reaction mixture containing 10.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 103). For the sample with excess of 1, the CD sensing was performed in duplicate. Then, the concentration and enantiomeric ratio were determined as 9.5 mM and 91.4:8.6 er using the protocol mentioned above (FIG. 104).

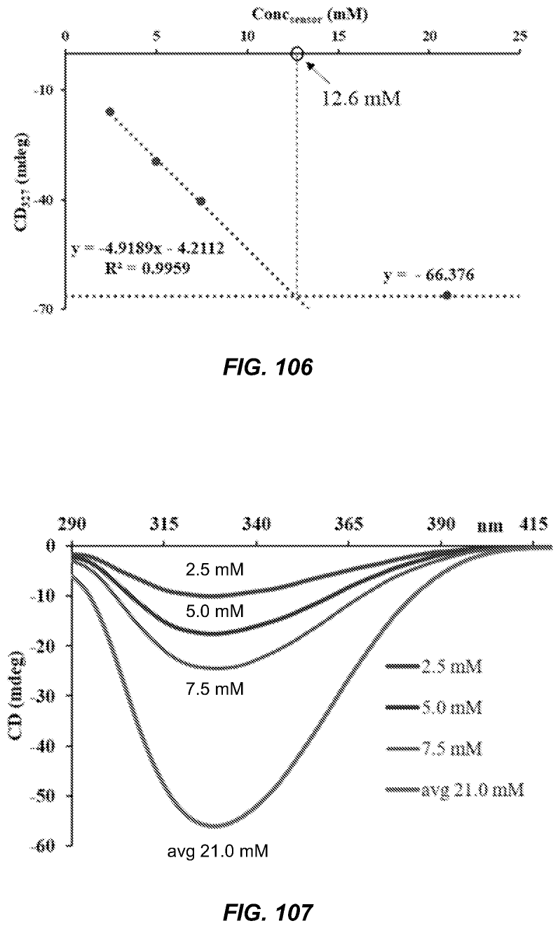

A sample containing enantioenriched L-Ala (85.0:15.0 er, 12.0 mM) was analyzed. First, a 0.1 M stock solution in water was prepared. To 120.0 μL of this solution were added varying volumes (25.0, 50.0, 75.0, and 210.0 μL) of probe 1 (0.1 M in acetonitrile) and 2.0 equiv. of K₂CO₃. The reaction mixture containing 12.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 105). For the sample with excess of 1, the CD sensing was performed in duplicate. Then, the concentration and enantiomeric ratio were determined as 12.6 mM and 85.1:14.9 er using the protocol mentioned above (FIG. 106).

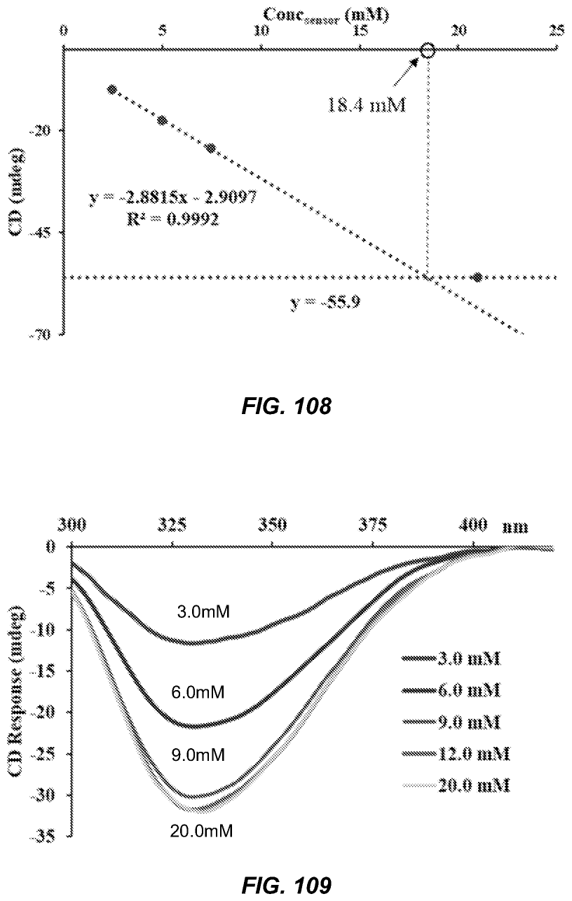

A sample containing enantioenriched L-Ala (70.0:30.0 er, 18.0 mM) was analyzed. First, a 0.1 M stock solution in water was prepared. To 180.0 μL of this solution were added varying volumes (25.0, 50.0, 75.0, and 210.0 μL) of probe 1 (0.1 M in acetonitrile) and 2.0 equiv. of K₂CO₃. The reaction mixture containing 18.0 mM of the amino acid was diluted to a total volume of 1.0 mL using water:ACN (6.5:3.5 v v) and stirred for 30 minutes. CD analysis was performed after diluting a 130.0 μL aliquot with 2.0 mL of ACN:water (4:1, v v) (FIG. 107). For the sample with excess of 1, the CD sensing was performed in duplicate. Then, the concentration and enantiomeric ratio were determined as 18.4 mM and 70.3:29.7 er using the protocol mentioned above (FIG. 108).

TABLE 3
Analysis of Absolute Configuration, er, and Concentration
of Alanine Samples with Probe 1 Using CD Sensing

| | |
|---|---|
| Sample composition | Sensing results |

| Abs | Conc | | Abs | Conc | |
|---|---|---|---|---|---|
| config. | (mM) | er | config. | (mM) | er |
| D | 19.0 | 97.5:2.5 | D | 19.5 | 97.4:2.6 |
| D | 17.0 | 88.0:12.0 | D | 17.3 | 88.1:11.9 |
| D | 15.0 | 76.0:24.0 | D | 15.3 | 75.0:25.0 |
| D | 13.0 | 66.0:34.0 | D | 12.5 | 65.8:34.2 |
| D | 11.0 | 55.0:45.0 | D | 10.9 | 54.7:45.3 |
| L | 20.0 | 55.0:45.0 | L | 20.8 | 54.8:45.2 |
| L | 18.0 | 70.0:30.0 | L | 18.4 | 70.3:29.7 |
| L | 12.0 | 85.0:15.0 | L | 12.6 | 85.1:14.9 |
| L | 14.0 | 80.5:19.5 | L | 13.9 | 79.8:20.2 |
| L | 10.0 | 91.0:9.0 | L | 9.5 | 91.4:8.6 |

### Example 8—Enzymatic Kinetic Resolution Sensing of Amine 12

A kinetic resolution of 1-phenylethylamine was performed following a literature protocol (Päiviö et al., “Solvent-Free Kinetic Resolution of Primary Amines Catalyzed by Candida Antarctica Lipase B: Effect of Immobilization and Recycling Stability,” Tetrahedron Asymm. 23:230-236 (2012), which is hereby incorporated by reference in its entirety).

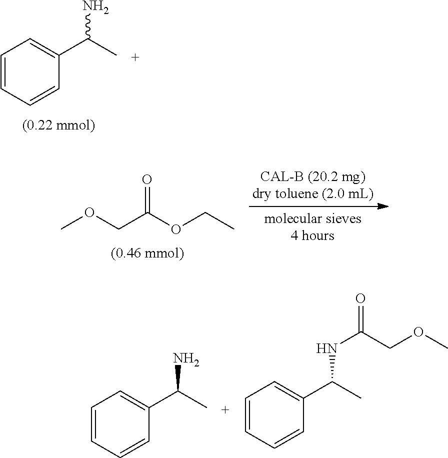

Ethyl methoxyacetate (0.46 mmol) was added into a reaction vessel containing molecular sieves (4 Å, 560.0 mg) and Candida antarctica lipase B (Cal B) (20.2 mg), anhydrous toluene (2.0 mL), and racemic 1-phenylethylamine (0.22 mmol, 110.0 mM). The reaction mixture was shaken at room temperature for 4 hours. To an aliquot of 150.0 μL of this solution were added varying volumes (30.0, 60.0, 90.0, 120.0, and 200 μL) of probe 1 (0.05 M in ACN) and 1.2 equivalents of triethylamine. The reaction mixtures were diluted to a total volume of 0.5 mL with ACN and stirred for 30 minutes. CD analysis was performed after diluting a 100.0 μL aliquot with 2.0 mL of CAN (FIG. 109).

The absolute configuration was determined as (S)-1-phenylethylamine using the sign of the Cotton effect. The experimentally obtained CD amplitudes at 324.0 nm were plotted against the concentrations of the sensor. Linear regression analysis using the CD amplitudes obtained with 1 in the region of excess of the analyte showed a linear increase. A horizontal line parallel to the x-axis (slope=0) representing the range where the CD amplitude is stagnant because the sensor is in excess of the amine analyte was obtained. The x-value at the intersection of these two lines was used to determine the concentration of the amine (keeping the sample dilution protocol described above in mind) as 9.41 mM in the CD sensing solutions. This corresponds to 28.5% (31.37 mM) in the reaction mixture.

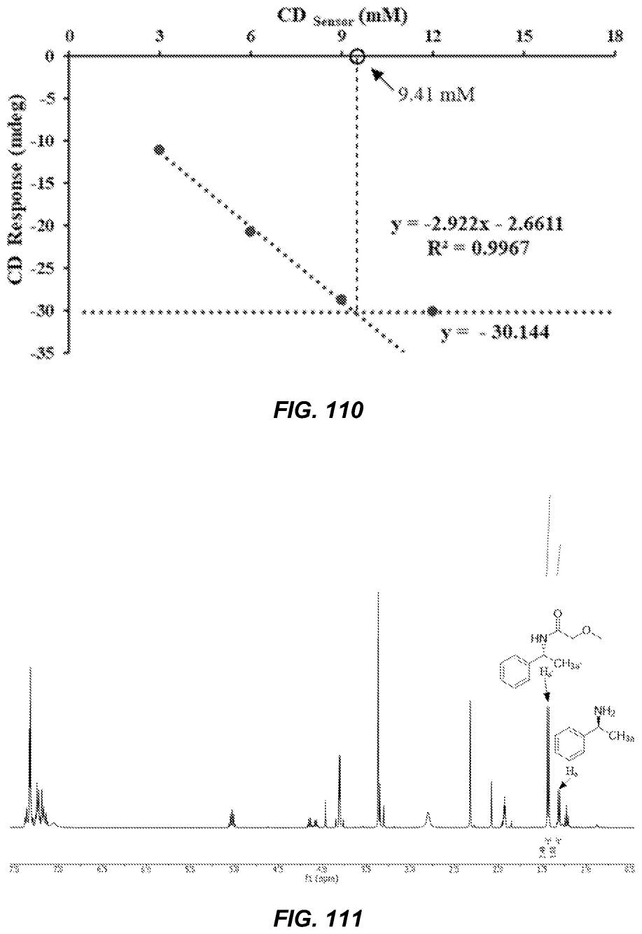

With the concentration of the analyte in hand, the enantiomeric composition was calculated by comparing the y-axis value (mdeg) to that of an enantiopure reference. This gave an enantiomeric ratio of 100.6:0 (FIG. 110).

¹H NMR spectroscopy was performed using a portion of the reaction mixture combined with d₃-ACN to determine the conversion of 1-phenylethylamine (FIG. 111). The ratio of 1-phenylethylamine to 2-methoxy-N-(1-phenylethyl)acetamide was calculated as 1:2.49. Thus, the remaining enantiopure amine in the reaction mixture was determined as 28.7% (31.52 mM).

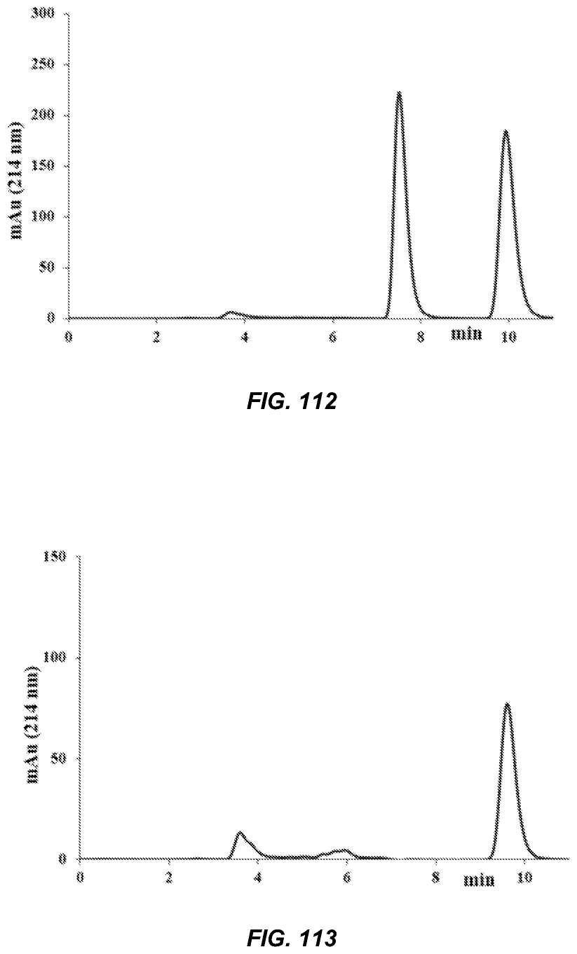

A portion of the crude reaction mixture (0.02 mmol) was derivatized with benzoyl chloride (1.0 equiv.) in the presence of 1.2 equivalent of triethylamine. N-(1-Phenylethyl)benzamide was isolated using preparative TLC and the enantiomeric excess was determined as >99% by chiral HPLC on a Chiralcel OD column using hexanes:IPA (80:20) as mobile phase, flow rate=1.0 mL/min, and UV detection at 214 nm (FIGS. 112-113).

### Discussion of Examples 1-8

The profound implications of small-molecule chirality in the chemical, materials, and life sciences are staggering and continue to shape technological innovation and developments (Brandt et al., “The Added Value of Small-Molecule Chirality in Technological Applications,” Nat. Rev. Chem. 1:45 (2017), which is hereby incorporated by reference in its entirety). The diversity of small chiral compounds that are routinely encountered in biological samples or used in chemical applications together with the constant search for methods amenable to micro-scaling and high-throughput screening methodology have directed increasing attention to chiroptical sensing systems (Leung et al., “Rapid Determination of Enantiomeric Excess: A Focus on Optical Approaches,” Chem. Soc. Rev. 41:448-479 (2012); Wolf et al., “Chirality Sensing Using Stereodynamic Probes with Distinct Electronic Circular Dichroism Output,” Chem. Soc. Rev. 42:5408-5424 (2013); Wu et al., “Chromogenic/Fluorogenic Ensemble Chemosensing Systems,” Chem. Rev. 115:7893-7943 (2015); Herrera et al., “Optical Analysis of Reaction Yield and Enantiomeric Excess. A New Paradigm Ready for Prime Time,” J. Am. Chem. Soc. 140:10385-10401 (2018); Li et al., “The Optoelectronic Nose: Colorimetric and Fluorometric Sensor Arrays,” Chem. Rev. 119:231-292 (2019), which are hereby incorporated by reference in their entirety). Chiral amines, amino alcohols, and amino acids are undoubtedly among the most important sensing targets and a robust chiroptical method that enables on-the-fly determination of the concentration and the enantiomeric composition of these compounds holds promise to accelerate the analytical workflow which often is the ultimate bottleneck in R&D endeavors. A broadly applicable chirality sensor that can manage the structural wealth of amines, amino alcohols, and amino acids as well as the drastically different chemical environments in which these typically occur would be extremely useful. In fact, chirality sensing of amines and amino alcohols usually needs to be performed in organic solvents while amino acids require a sensor that can operate in aqueous solutions, especially if biological samples are analyzed (Pagliari et al., “Enantioselective Fluorescence Sensing of Amino Acids by Modified Cyclodextrins: Role of the Cavity and Sensing Mechanism,” Chem. Eur. J. 10:2749-2758. (2004); Wolf et al., “An Enantioselective Fluorescence Sensing Assay for Quantitative Analysis of Chiral Carboxylic Acids and Amino Acid Derivatives,” Chem. Commun. 4242-4244 (2006); Mei et al., “Synthesis of a Sterically Crowded Atropisomeric 1,8-Diacridylnaphthalene for Dual Mode Enantioselective Fluorosensing,” J. Org. Chem. 71:2854-2861 (2006); Mei et al., “Determination of Enantiomeric Excess and Concentration of Unprotected Amino Acids, Amines, Amino Alcohols, and Carboxylic Acids by Competitive Binding Assays with a Chiral Scandium Complex,” J. Am. Chem. Soc. 128:13326-13327 (2006); Liu et al., “Enantioselective Fluorescence Sensing of Chiral α-Amino Alcohols,” J. Org. Chem. 73:4267-4270 (2008); Liu et al., “Highly Enantioselective Fluorescent Recognition of Serine and Other Amino Acid Derivatives,” Org. Lett. 12:4172-4175 (2010); He et al., “Enantioselective Recognition of α-Hydroxycarboxylic Acids and N-Boc-Amino Acids by Counterion-Displacement Assays with a Chiral Nickel(II) Complex,” Org. Lett. 13:804-807 (2011); He et al., “Determination of Concentration and Enantiomeric Excess of Amines and Amino Alcohols with a Chiral Nickel(II) Complex,” Chem. Commun. 47:11641-11643 (2011); Minami et al., “Turn-on” Fluorescent Sensor Array For Basic Amino Acids in Water,” Chem. Commun. 50:61-63 (2014); Huang et al., “Zn(II) Promoted Dramatic Enhancement in the Enantioselective Fluorescent Recognition of Functional Chiral Amines by a Chiral Aldehyde,” Chem. Sci. 5:3457-3462 (2014); Shcherbakova et al., “Determination of Enantiomeric Excess in Amine Derivatives with Molecular Self-Assemblies,” Angew. Chem. Int. Ed. 54:7130-7133 (2015); Feng et al., “Fluorescence Turn-on Enantioselective Recognition of Both Chiral Acidic Compounds and α-Amino Acids by a Chiral Tetraphenylethylene Macrocycle Amine,” J. Org. Chem. 80:8096-8101 (2015); Wen et al., “Rational Design of a Fluorescent Sensor to Simultaneously Determine Both the Enantiomeric Composition and the Concentration of Chiral Functional Amines,” J. Am. Chem. Soc. 137:4517-4524 (2015); Shcherbakova et al., “Toward Fluorescence-Based High-Throughput Screening for Enantiomeric Excess in Amines and Amino Acid Derivatives,” Chem. Eur. J. 22:10074-10080 (2016); Wang et al., “Enantioselective Fluorescent Recognition of Amino Acids by Amide Formation: An Unusual Concentration Effect,” J. Org. Chem. 82:12669-12673 (2017); Zeng et al., “Enantioselective Fluorescent Imaging of Free Amino Acids in Living Cells,” Chem. Eur. J. 23:2432-2438 (2017); Zhu et al., “Free Amino Acid Recognition: A Bisbinaphthyl-Based Fluorescent Probe with High Enantioselectivity,” J. Am. Chem. Soc. 141:175-181 (2019); Bentley et al., “A Stereodynamic Chemosensor with Selective Circular Dichroism and Fluorescence Readout for In Situ Determination of Absolute Configuration, Enantiomeric Excess and Concentration of Chiral Compounds,” J. Am. Chem. Soc. 135:12200-12203 (2013); Bentley et al., “Chirality Sensing of Amines, Diamines, Amino Acids, Amino Alcohols, and α-Hydroxy Acids with a Single Probe,” J. Am. Chem. Soc. 135:18052-18055 (2013); Bentley et al., “Comprehensive Chirality Sensing: Development of Stereodynamic Probes with a Dual (Chir)optical Response,” J. Org. Chem. 79:6517-6531 (2014); Shirbhate et al., “Optical and Fluorescent Dual Sensing of Aminoalcohols by in Situ Generation of BODIPY-like Chromophore,” J. Am. Chem. Soc. 142:4975-4979 (2020); Huang et al., “Zinc Porphyrin Tweezer in Host-Guest Complexation: Determination of Absolute Configurations of Diamines, Amino Acids, and Amino Alcohols by Circular Dichroism,” J. Am. Chem. Soc. 120:6185-6186 (1998); Folmer-Andersen et al., “Colorimetric Enantiodiscrimination of Alpha-Amino Acids in Protic Media,” J. Am. Chem. Soc. 127:7986-7987 (2005); Folmer-Andersen et al., “Pattern-Based Discrimination of Enantiomeric and Structurally Similar Amino Acids: An Optical Mimic of the Mammalian Taste Response,” J. Am. Chem. Soc. 128:5652-5653 (2006); Kim et al., “Highly Stereospecific Generation of Helical Chirality by Imprinting with Amino Acids: A Universal Sensor for Amino Acid Enantiopurity,” Angew. Chem. Int. Ed. 47:8657-60 (2008); Leung et al., “Transitioning Enantioselective Indicator Displacement Assays for α-Amino Acids to Protocols Amenable to High-Throughput Screening,” J. Am. Chem. Soc. 130:12328-12333 (2008); Joyce et al., “Enantio- and Chemoselective Differentiation of Protected α-Amino Acids and β-Homoamino Acids with a Single Copper(II) Host,” Chem. Eur. J. 18:8064-8069 (2012); Scaramuzzo et al., “Determination of Amino Acid Enantiopurity and Absolute Configuration: Synergism Between Configurationally Labile Metal-Based Receptors and Dynamic Covalent Interactions,” Chem. Eur. J. 19:16809-16813 (2013); Seifert et al., “Exploitation of the Majority Rules Effect for the Accurate Measurement of High Enantiomeric Excess Values Using CD Spectroscopy,” Chem. Commun. 50:15330-15332 (2014); Biedermann et al., “Noncovalent Chirality Sensing Ensembles for the Detection and Reaction Monitoring of Amino Acids, Peptides, Proteins, and Aromatic Drugs,” Angew. Chem. Int. Ed. 53:5694-5699 (2014); Bentley et al., “Miniature High-Throughput Chemosensing of Yield, Ee and Absolute Configuration From Crude Reaction Mixtures,” Science Adv. 2:e1501162 (2016); De los Santos et al., “Chiroptical Asymmetric Reaction Screening via Multicomponent Self-Assembly,” J. Am. Chem. Soc. 138:13517-13520 (2016); Badetti et al., “Multimetallic Architectures from the Self-assembly of Amino Acids and Tris(2-pyridylmethyl)amine Zinc(II) Complexes: Circular Dichroism Enhancement by Chromophores Organization,” Chem. Eur. J. 22:6515-6518 (2016); Pilicer et al., “Biomimetic Chirality Sensing with Pyridoxal-5”-phosphate, “J. Am. Chem. Soc. 139:1758-1761 (2017); Thanzeel et al.,” Substrate-Specific Amino Acid Sensing Using a Molecular D/L-Cysteine Probe for Comprehensive Stereochemical Analysis in Aqueous Solution,” Angew. Chem. Int. Ed. 56:7276-7281 (2017); Zardi et al., “Concentration-Independent Stereodynamic g-Probe for Chiroptical Enantiomeric Excess Determination,” J. Am. Chem. Soc. 139:15616-15619 (2017); Thanzeel et al., “Click Chemistry Enables Quantitative Chiroptical Sensing of Chiral Compounds in Protic Media and Complex Mixtures,” Nat. Comm. 9:5323 (2018); Lynch et al., “Chiroptical Sensing of Unprotected Amino Acids, Hydroxy Acids, Amino Alcohols, Amines and Carboxylic Acids with Metal Salts,” Chem. Commun. 55:6297-6300 (2019); Thanzeel et al., “Quantitative Chiroptical Sensing of Free Amino Acids, Biothiols, Amines, and Amino Alcohols with an Aryl Fluoride Probe,” J. Am. Chem. Soc. 141:16382-16387 (2019), which are hereby incorporated by reference in their entirety).

To date, chiral amines and amino alcohols have found numerous industrial applications, for example as essential structural motifs in agrochemicals and pharmaceuticals (Ager et al., “1,2-Amino Alcohols and Their Heterocyclic Derivatives as Chiral Auxiliaries in Asymmetric Synthesis,” Chem. Rev. 96:835-876 (1996); Nugent et al., “Chiral Amine Synthesis: Methods, Developments and Applications,” Wiley-VCH: Weinheim, Germany (2010), which are hereby incorporated by reference in their entirety). The widespread use and occurrence of R- and S-amino acids in chemistry (List et al., “Proline-Catalyzed Direct Asymmetric Aldol Reactions,” J. Am. Chem. Soc. 122:2395-2396 (2000); Movassaghi et al., “The Simplest ‘Enzyme’,” Science, 298:1904-1905 (2002); Klussmann et al., “Thermodynamic Control of Asymmetric Amplification in Amino Acid Catalysis,” Nature 441:621-623 (2006); Coulthard et a., “Stereocontrolled Organocatalytic Synthesis of Prostaglandin PGF2α in Seven Steps,” Nature 489:278-281 (2012); Fujiwara et al., “Total Synthesis of (−)-Acetylaranotin,” Angew. Chem. Int. Ed. 51:13062-13065 (2012); Mercado-Marin et al., “Total Synthesis and Isolation of Citrinalin and Cyclopiamine Congeners,” Nature 509:318-324 (2014); Ruchti et al., “Ir-Catalyzed Reverse Prenylation of 3-Substituted Indoles: Total Synthesis of (+)-Aszonalenin and (−)-Brevicompanine B,” J. Am. Chem. Soc. 136:16756-16759 (2014); Matthies et al., “Total Synthesis of Legionaminic Acid as Basis for Serological Studies,” J. Am. Chem. Soc. 137:2848-2851 (2015), which are hereby incorporated by reference in their entirety) and biology (Friedman M., “Origin, Microbiology, Nutrition, and Pharmacology of D-Amino Acids,” Chem. Biodivers. 7:1491-1530 (2010); Bruckner et al., (Eds.) “D-Amino Acids in Chemistry, Life Sciences, and Biotechnology,” John Wiley & Sons, New York (2011); Friedman et al., “Nutritional and Medicinal Aspects of D-Amino Acids,” Amino Acids. 42:1553-1582 (2012); Simon et al., “D-Amino Acid Scan of Two Small Proteins,” J. Am. Chem. Soc. 138:12099-12111 (2016), which are hereby incorporated by reference in their entirety) are particularly noteworthy. Serine, aspartic acid, alanine, and cysteine exist in the form of nonracemic mixtures in the central nervous system and endocrine organs of humans and mammals (Hamase et al., “D-Amino Acids in Mammals and their Diagnostic Value,” J. Chromatogr. B 781:73-91 (2002); Yoshimura et al., “D-Amino Acids in the Brain: Structure and Function of Pyridoxal Phosphate-Dependent Amino Acid Racemases,” FEBSJ. 275:3527-3537 (2008); Fuchs et al., “D-Serine: The Right or Wrong Isoform?,” Brain Res. 1401:104-117 (2011); Billard J.-M., “D-Amino Acids in Brain Neurotransmission and Synaptic Plasticity,” Amino Acids 43:1851-1860 (2012); Yamanaka et al., “D-Amino Acids in the Brain and Mutant Rodents Lacking D-Amino-Acid Oxidase Activity,” Amino Acids 43:1811-1821 (2012); Kiriyama et al., “D-Amino Acids in the Nervous and Endocrine Systems,” Scientifica 6494621 (2016), which are hereby incorporated by reference in their entirety). The concentration and enantiomeric composition of these and maybe other amino acids appear to play a role in schizophrenia, Parkinson's, Huntington's, Alzheimer's and other neurological diseases and carry therefore significant value as diagnostic biomarkers (Fuchs et al., “D-Amino Acids in the Central Nervous System in Health and Disease,” Mol. Genet. Metab. 85:168-180 (2005); Visser et al., “A Sensitive and Simple Ultra-High-Performance-Liquid Chromatography-Tandem Mass Spectrometry Based Method for the Quantification of D-Amino Acids in Body Fluids,” J. Chromatogr. A 1218:7130-7136 (2011); Weathertly et al., “D-Amino Acid Levels in Perfused Mouse Brain Tissue and Blood: A Comparative Study,” ACS Chem. Neurosci. 8:1251-1261 (2017), which are hereby incorporated by reference in their entirety).

The chiroptical analysis of chiral compounds is generally performed with chemical sensors that generate a dual spectroscopic response as a result of carefully designed molecular recognition and binding events. Several assays that allow determination of analyte concentration based on characteristic UV or fluorescence spectroscopic signals and of the enantiomeric ratio (er) by induced circular dichroism (CD) readouts are known and have been proven to be widely useful (Herrera et al., “Optical Analysis of Reaction Yield and Enantiomeric Excess. A New Paradigm Ready for Prime Time,” J. Am. Chem. Soc. 140:10385-10401 (2018), which is hereby incorporated by reference in its entirety). These methods have in common that the total analyte concentration must be determined first and this information is then applied in the calculation of the er, an elaborate stepwise process that requires two calibration curves. A unified sensing methodology that achieves both tasks at once but solely relies on CD measurements would significantly simplify sample handling and streamline the operational workflow.

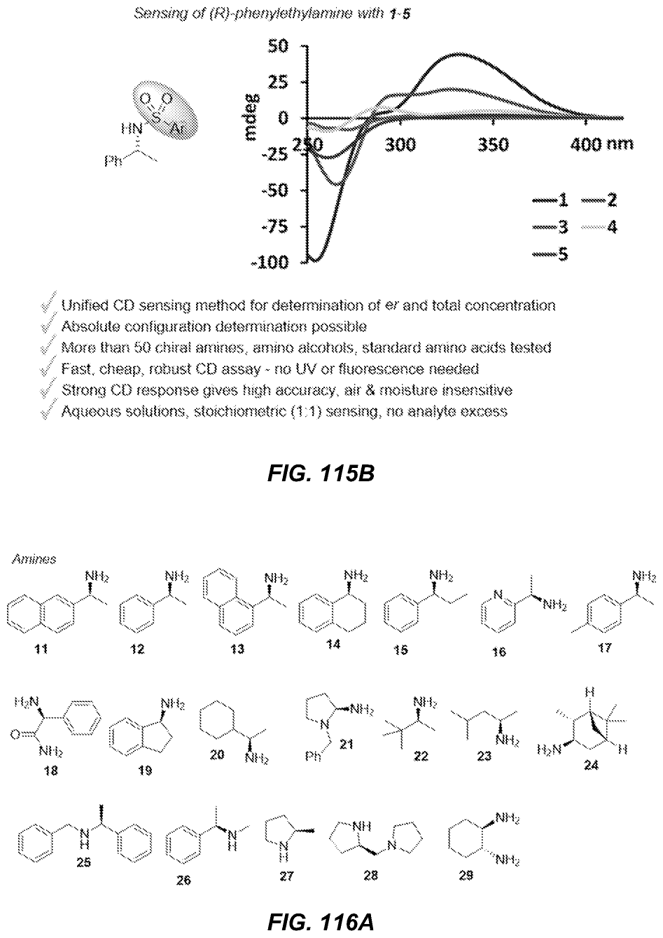

A series of arenesulfonyl chloride probes 1-10 were investigated as potential universally useful chirality sensor that is readily available and accomplish fast concentration and er analysis of minute amounts of amines, amino alcohols, and amino acids in either organic solvents or aqueous solutions. Initial testing with aliphatic and aromatic chiral amines showed that the reaction with stoichiometric amounts of 2-nitrobenzenesulfonyl chloride, 1, gives strong Cotton effects at long wavelengths which are generally considered advantageous features for quantitative sensing applications (FIGS. 115A-B). The sulfonamide formation occurs quantitatively at room temperature and is complete within a few minutes according to NMR and CD reaction monitoring. Moreover, the fact that the sensor can be used in aqueous solutions indicated that amino acid sensing might also be possible.

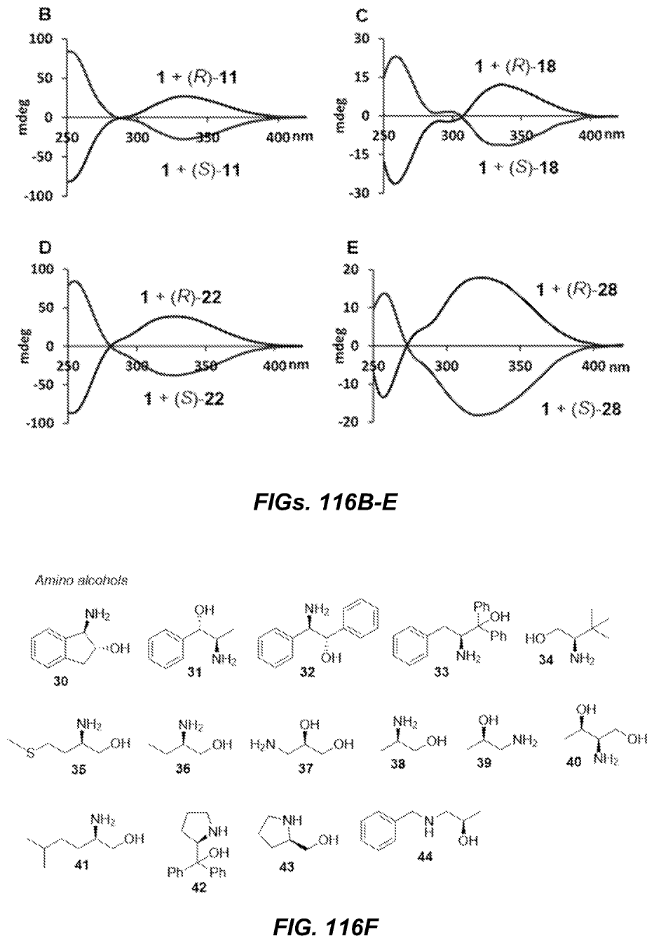

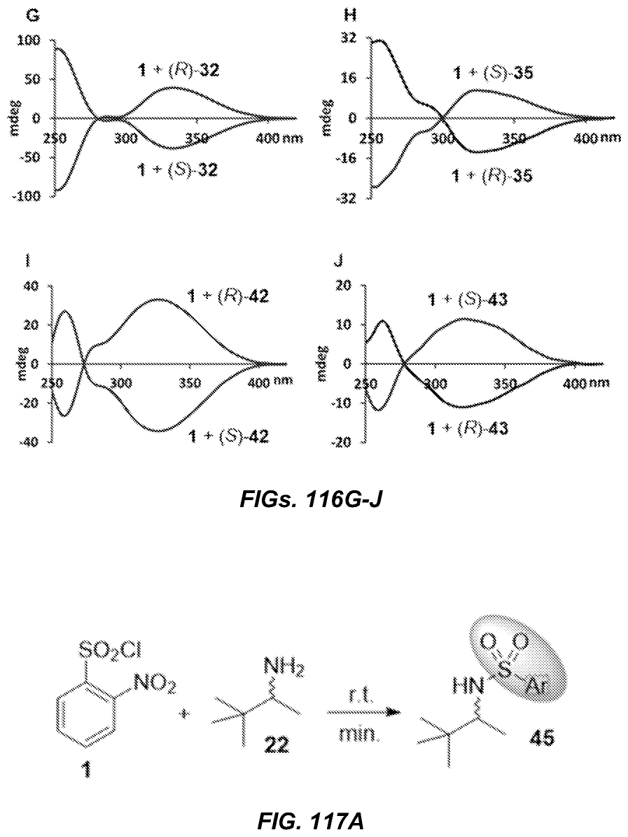

The favorable chemical and optical features of the arylsulfonyl probes encouraged the investigation of chirality sensing with a large variety of more than thirty amines and amino alcohols (FIGS. 116A-J). Although other arylsulfonyl chlorides can also be used, compound 1 was selected for this purpose based on initial screening results. The CD sensing of both aromatic and aliphatic amines 11-29 via sulfonamide bond formation yielded strong CD effects at low concentrations. The successful chiroptical sensing of the secondary amines 25-28 is noteworthy because it extends beyond the scope of Schiff base sensors that have been introduced in recent years. Similarly, sensing of all amino alcohols 29-43 gave CD outputs sufficient for quantitative analysis which underscores the general usefulness of 1.

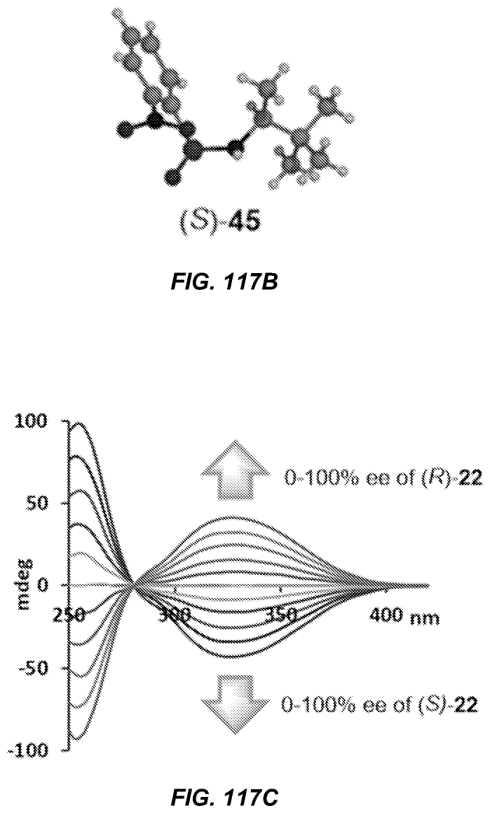

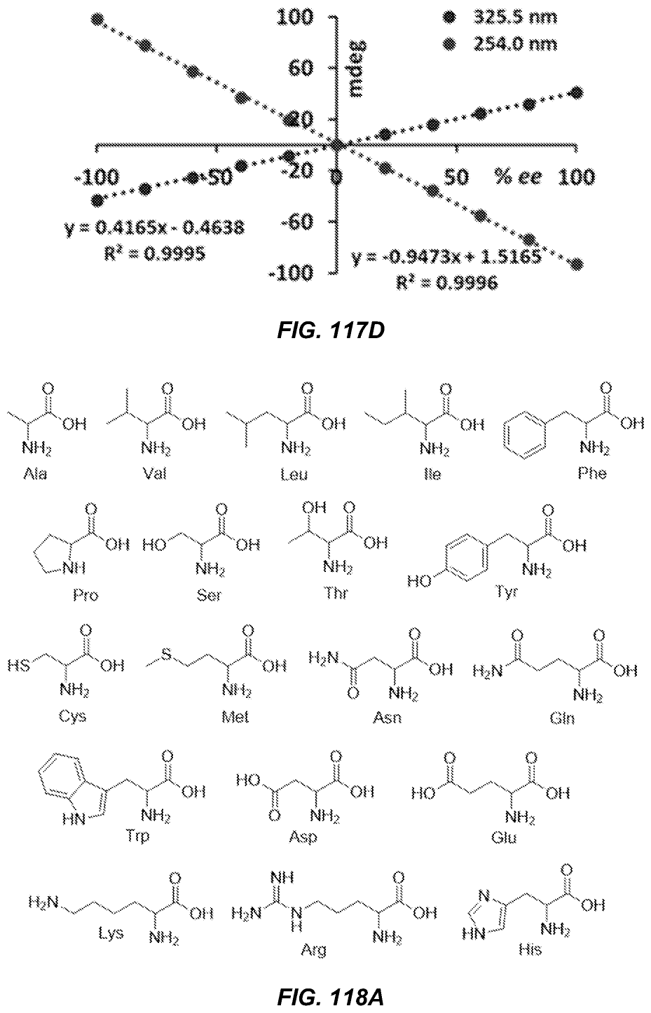

Using 3,3-dimethylbutan-2-amine, 22, a simple aliphatic amine that is generally considered one of the more challenging substrates, as model target compound the change in the CD response of probe 1 upon binding of samples with varying enantiomeric composition was investigated. The CD maxima at 254 and 325 nm was found to increase linearly with the er values of 22 (FIG. 117A-D). The operational simplicity of chirality sensing with 1 and the strong, linear CD responses to nonracemic samples of 22 as well as the wide application spectrum were observed. The target compound and the probe were simply combined in the presence of a base under air and the mixture was subjected to CD analysis after a few minutes of stirring. Additional precautions including the use of anhydrous solvents were not necessary. 2-Nitrobenzenesulfonyl chloride is readily available, inexpensive, and bench-stable which further underscores the practicality of this chiroptical sensing assay.

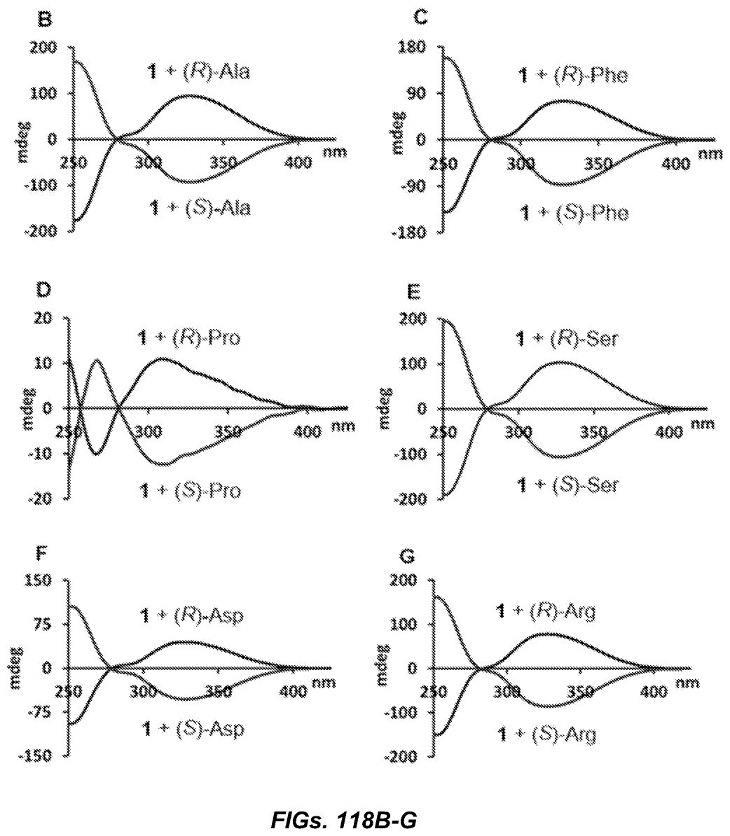

Compound 1 was applied to all nineteen chiral standard amino acids (FIG. 118A-G). Without exception, strong CD signals above 300 nm were recorded, a stark contrast to previously reported optical amino acid sensors that are not equally applicable to the different subclasses of amino acids. Representative examples of amino acid displaying the whole range of aliphatic and aromatic nonpolar, polar, basic, and acidic side chains were used for chirality sensing. The weakest CD responses of 1 were observed with the enantiomers of proline which gave induced CD amplitudes slightly above 10 mdeg at 0.54 mM in ACN/borate buffer solutions. However, with near-racemic samples that significantly smaller signal intensities suffice for quantitative concentration and er sensing. Further comparison of the CD outputs revealed that the sensing of all R-amino acids give a positive Cotton effect while the opposite results are obtained with the S-enantiomers. The only exception was cysteine. This, however, does not reflect a change in the chiroptical recognition pattern of 1 but is an artificial consequence of the CIP atom priority nomenclature. The consistent correlation of the sign of the measured CD effects with the absolute configuration of the 19 amino acids showed the use of 1 for chiroptical stereochemical assignments which remains an important endeavor, in particular in the pharmaceutical development world.

The outstanding chiroptical properties, wide application scope, and the operational simplicity of chiral compound sensing with 1 lead to investigation of the possibility of simultaneous concentration and er determination solely based on CD measurements—a highly sought-after goal that has not been accomplished previously (Wen et al., “Rational Design of a Fluorescent Sensor to Simultaneously Determine Both the Enantiomeric Composition and the Concentration of Chiral Functional Amines,” J. Am. Chem. Soc. 137:4517-4524 (2015), which is hereby incorporated by reference in its entirety). Eliminating the need to perform additional UV or fluorescence experiments, which are used to quantify the total sample amount while er values are obtained from CD data, would significantly streamline the workflow. Typically, quantitative UV/CD and fluorescence/CD sensing requires the use of two calibration curves, separate sample preparation or dilution, and additional precautions, for example degassing of solvents and solutions to avoid interference of dioxygen with fluorescence quenching measurements. As a result, state-of-the-art optical chirality sensing can be labor intensive, cumbersome, and prone to errors. These drawbacks most likely are among the major reasons why this field has not yet found widespread acceptance for use in R&D and quality control applications. In contrast to other widely used spectroscopies, including NMR, IR, UV, and fluorescence, circular dichroism is a superior choice for quantitative analysis of chiral compounds because it is the only one among the aforementioned techniques with an inherent capacity to differentiate between enantiomers. This merits several advantages that are extremely practical, for example the simplicity of using a readily available, inexpensive achiral probe like 1 rather than chiral derivatizing or solvating agents that have to be prepared in enantiopure form to achieve enantiodifferentiation and er quantification via formation of diastereomers.

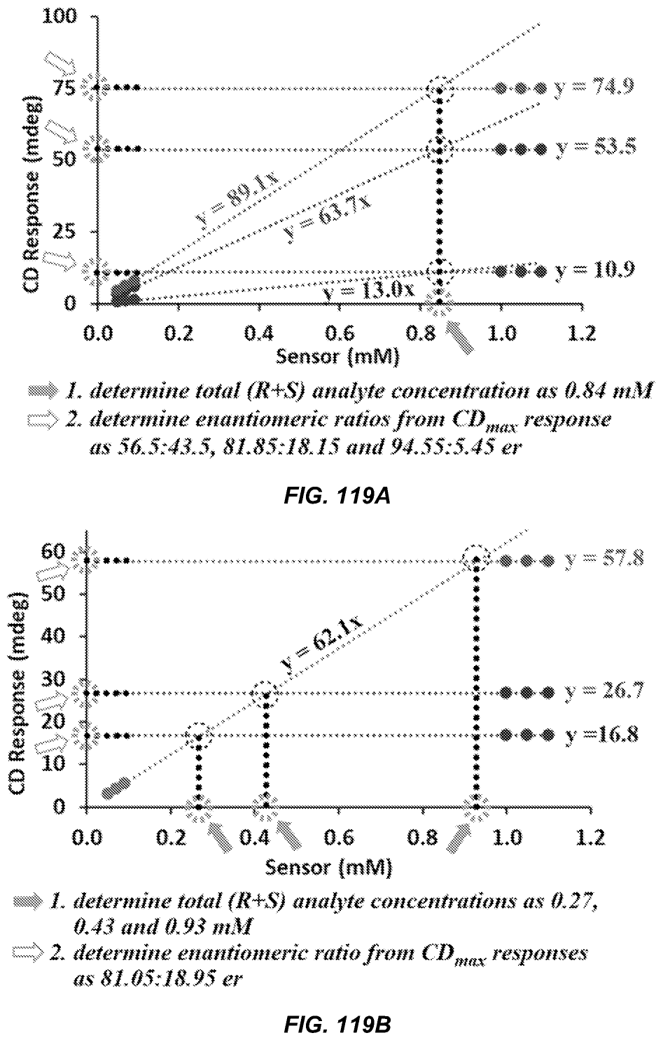

The present application describes a strategy that can be easily adapted to determine both the total amount and the enantiomeric composition of chiral compounds solely by CD sensing with a straightforward mix-and-measure protocol and simple data processing. It was assumed that the treatment of a chiral sample, for example an amine or one of the amino acids, of unknown concentration and varying er with a) relatively small but steadily increasing probe amounts and b) excess of the probe would allow to obtain both variables by plotting the recorded CD intensities versus the sensor concentration for linear regression analysis. The underlying principle of this approach was first exemplified with imaginary samples covering wide concentration and er ranges and then with actual test example of an amine and an amino acid (FIGS. 119A-B and 120A-B). It seemed most practical to use CD maxima for this purpose although readings at other wavelengths or of the whole spectrum could in principle be utilized to further increase the accuracy and robustness of the method if desirable. The analysis was accomplished with four CD measurements conducted in parallel. This analysis is practical as the parallel generation of several chiroptical sensing responses per sample is routine and can be easily accomplished with modern multi-well plate instrumentation and automated CD reader equipment.

Considering the distinct circular dichroism responses of probe 1 to the chiral target compounds screened above, the following rationale for combined concentration and enantiomeric ratio (er) analysis was developed. For simplicity it was assumed that the CD sensing of a 1.0 mM sample of an enantiopure analyte A with probe 1 would give a maximum of 100.0 mdeg in the CD spectrum. The sensing of an enantiopure sample containing the same target compound at 0.84 mM would then be expected to produce the same CD curve but with a signal intensity of only 84.0 mdeg. Because of the linear dependence of the induced CD signal on the analyte enantiomeric composition and concentration, respectively, one can calculate that the sensing of a sample of 81.85:18.15 er (63.7% ee) at 0.84 mM with an equimolar probe amount would give a CD signal of 53.5 mdeg. Because only 0.84 mM analyte A is present in the sensing solution one would obtain the same CD intensity independent of the amount of excess of 1. In other words, the induced CD maximum would always have to be 53.5 mdeg when aliquots of the 0.84 mM sample having 81.85:18.15 er are treated with 1.00, 1.05, or 1.10 mM of the probe. The plotting of these CD values versus the sensor concentration would therefore give a straight line parallel to the x-axis (FIG. 119A, horizontal line, y=53.5). By contrast, the addition of small but steadily increasing probe amounts (0.05, 0.07, 0.09 mM) would consume only a fraction of the analyte present in the 0.84 mM sample. Plotting of the corresponding CD amplitudes against the sensor concentration would give a straight line with a slope indicative of the sample er (FIG. 119A, line with y=63.7x). Importantly, enantiodiscrimination processes, for example kinetic resolution, that would affect the analysis are not a concern and can be excluded because the arylsulfonyl chloride 1 is achiral, unlike chiral solvating and derivatizing agents widely used in other optical assays and NMR methods. Thus, the actual sample concentration can be determined from the projection of the intersection of the two lines to the x-axis as 0.84 mM. With this value in hand, one can then easily calculate the sample er using the known CDₘₐₓ response of 84.0 mdeg that the sensing of an enantiopure analyte would produce. Because the CD maximum observed with excess of 1 is only 53.5 mdeg (intersection of the horizontal line with the y-axis) one can calculate that the er of A must be 81.85:18.15. This concept is visualized in FIG. 119A for two other samples of the same total concentration ([R]+[S]=0.84 mM) but with 56.5:43.5 er (13.0% ee) and 94.55:5.45 er (89.1% ee), respectively. The analysis of the sample with the relatively low er yielded a straight line with a small slope (y=13.0 x) and a horizontal line obtained with excess probe described by y=10.9. The projection of the intersection of these two lines to the x-axis (dashed circle) gave the sample concentration of 0.84 mM while the y-value yields the sample er. The steepest slope (y=89.1 x) was obtained with the most enantioenriched sample which, of course, intercepts with the horizontal line (y=74.9) at x=0.84 mM. These three examples show that samples with the same concentration will yield the same x-axis value at the interception point of the two lines while higher enantiomeric ratios give steeper slopes.

Three samples of A that have the same enantiomeric composition (81.05:18.95 er, 62.1% ee) but different concentrations of 0.27, 0.43 and 0.93 mM, respectively, were then considered (FIG. 119B). Because these samples exhibit the same er the CD sensing protocol with small but steadily increasing amounts of 1 would afford the exact same slope (y=62.1 x). But individual horizontal lines were generated because the CD responses that one would have measured with excess of the probe correspond to the individual sample concentrations that can easily be obtained as outlined above from the projection of the line interceptions to the x-axis (The slopes of the lines obtained with low probe amounts were the same as the % ee values of the samples. This was the case because the maximal induced CD signal of a 1.0 mM sample was set as 100.0 mdeg).

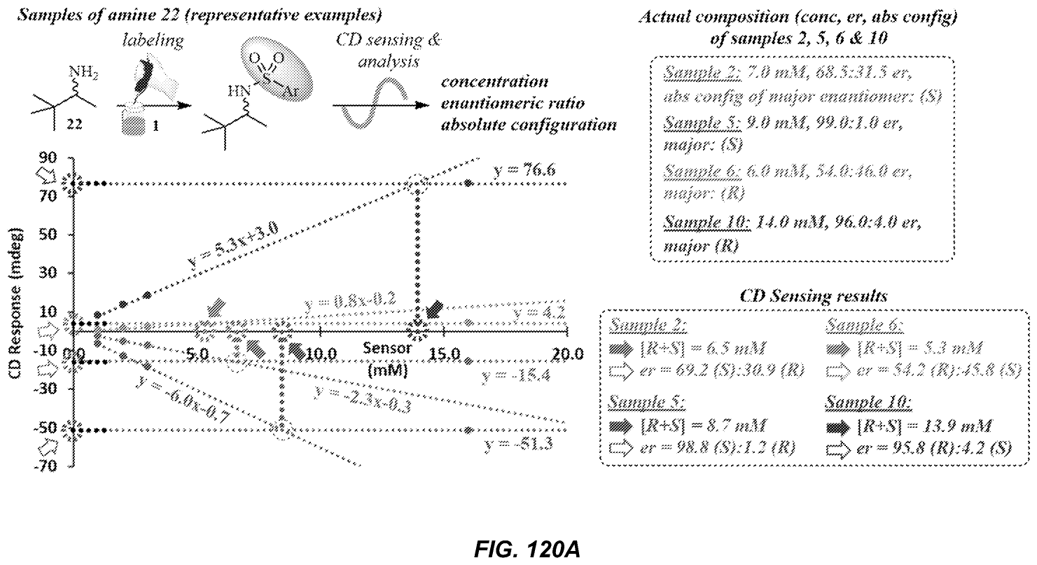

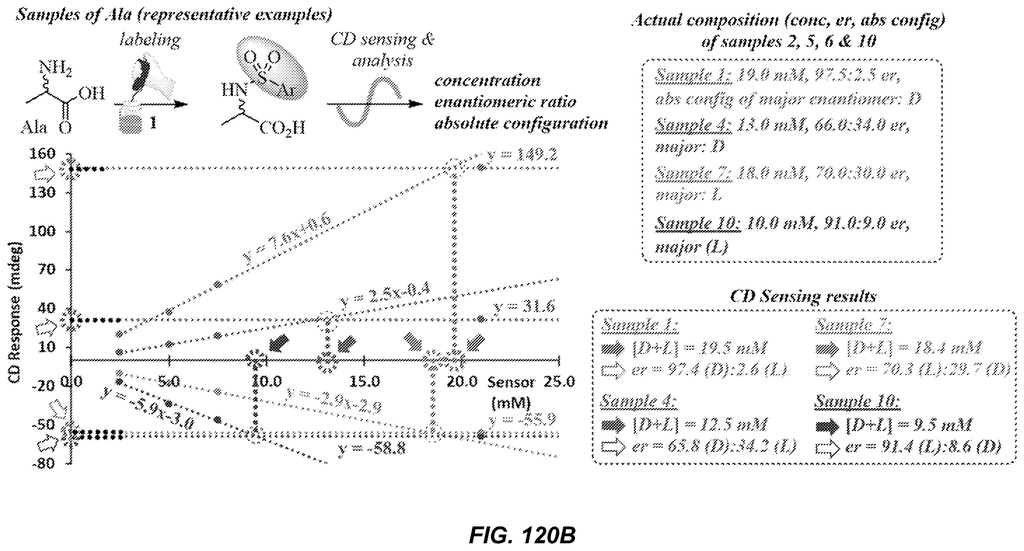

This concept was tested with randomly prepared 3,3-dimethylbutan-2-amine and alanine samples of varying concentrations and enantiomeric compositions (FIGS. 120A-B). First, a total of ten samples containing 5.0-14.0 mM of the amine 22 in low or high enantiomeric ratios and excess of either the R- or S-enantiomer were analyzed. To aliquots of each sample were added 1.0, 2.0, 3.0, and 16.0 mM of the sulfonyl chloride probe to generate a total of four sensing mixtures that were then diluted and subjected to fast CD measurements. The signal intensities obtained at 324.0 nm were plotted against the individual sensor concentrations following the strategy described above. The graphical analysis of four examples is shown in FIG. 120A and the results for all 10 samples are listed in Table 4. This unified CD sensing strategy provided more accurate data than previously reported chirality sensing methods that require a combination of UV and CD analysis to achieve concentration and er determination, respectively. For example, when CD sensing protocol was applied to sample #2 (7.0 mM, er: 68.5 (S):31.5 (R)), 6.5 mM and 69.2 (S):30.9 (R) were calculated, which shows that the concentration and er determinations are in excellent agreement with the actual values. Similarly, CD sensing of sample #10 (14.0 mM, er: 96.0 (R):4.0 (S)) gave 13.9 mM and 95.8 (R):4.2 (S), see entries 2 and 10 in Table 4.

TABLE 4
Unified CD Sensing of the Absolute Configuration, er, and
Concentration of 3,3-dimethylbutylamine Samples with 1

| | Sample composition | | | CD Sensing results | | |
|---|---|---|---|---|---|---|
| Sample | Abs | Conc. | Enantiomer | Abs | Conc. | Enantiomer |
| No. | config. | (mM) | ratio | config. | (mM) | ratio |
| 1 | S | 5.0 | 55.0:45.0 | S | 4.0 | 55.5:45.5 |
| 2 | S | 7.0 | 68.5:31.5 | S | 6.5 | 69.2:30.9 |
| 3 | S | 10.0 | 85.0:15.0 | S | 10.3 | 84.6:15.4 |
| 4 | S | 13.0 | 52.5:47.5 | S | 12.7 | 52.4:47.6 |
| 5 | S | 9.0 | 99.0:1.0 | S | 8.7 | 98.8:1.2 |
| 6 | R | 6.0 | 54.0:46.0 | R | 5.3 | 54.2:45.8 |
| 7 | R | 8.0 | 81.5:18.5 | R | 7.6 | 81.9:18.1 |
| 8 | R | 12.0 | 92.0:8.0 | R | 12.2 | 92.1:7.9 |
| 9 | R | 14.0 | 65.0:35.0 | R | 13.6 | 64.8:35.2 |
| 10 | R | 14.0 | 96.0:4.0 | R | 13.9 | 95.8:4.2 |

The unified CD sensing method was then applied to ten solutions containing 10.0-20.0 mM of alanine in substantially varying enantiomeric compositions and excess of either the L- or D-enantiomer. The detailed analysis of samples 1, 4, 7, and 10 is shown in FIG. 120B and the results for all D/L alanine mixtures are listed in Table 5. The unified CD sensing of sample #1 (19.0 mM, er: 97.5 (D):2.5 (L)) gave 19.5 mM and 97.4 (D):2.6 (L) which underscores the high accuracy of simultaneous concentration and er determination with this protocol. This is the case with all samples and comparison of the actual sample compositions and the chirality sensing results revealed that deviations are consistently within a small error margin. For example, CD sensing of sample #10 (10.0 mM, er: 91.0 (L):9.0 (D)) gave 9.5 mM and 91.4 (L):8.6 (D), see entry 10 in Table 5. The accuracy of this method may be further improved, if desirable, by using automated sample preparation and pipetting equipment. As stated above, parallel CD sensing of aliquots of chiral samples with different sensor amounts can easily be conducted with modem CD plate readers. This generally available technology and this practical sensing assay streamline for the first time combined concentration and er analysis that can now be exclusively based on CD measurements. Because the CD output generated by 1 increases linearly with the enantiomeric sample composition and with the concentration of the chiral target compound one does not need to generate a separate calibration curve.

TABLE 5
Unified CD Sensing of Absolute Configuration,
er, and Concentration of Alanine Samples with 1

| | Sample composition | | | Sensing results | | |
|---|---|---|---|---|---|---|
| Sample | Abs | Conc. | Enantiomer | Abs | Conc. | Enantiomer |
| No. | config. | (mM) | ratio | config. | (mM) | ratio |
| 1 | D | 19.0 | 97.5:2.5 | D | 19.5 | 97.4:2.6 |
| 2 | D | 17.0 | 88.0:12.0 | D | 17.3 | 88.1:11.9 |
| 3 | D | 15.0 | 76.0:24.0 | D | 15.3 | 75.0:25.0 |
| 4 | D | 13.0 | 66.0:34.0 | D | 12.5 | 65.8:34.2 |
| 5 | D | 11.0 | 55.0:45.0 | D | 10.9 | 54.7:45.3 |
| 6 | L | 20.0 | 55.0:45.0 | L | 20.8 | 54.8:45.2 |
| 7 | L | 18.0 | 70.0:30.0 | L | 18.4 | 70.3:29.7 |
| 8 | L | 12.0 | 85.0:15.0 | L | 12.6 | 85.1:14.9 |
| 9 | L | 14.0 | 80.5:19.5 | L | 13.9 | 79.8:20.2 |
| 10 | L | 10.0 | 91.0:9.0 | L | 9.5 | 91.4:8.6 |

Finally, probe 1 and the unified CD sensing concept were used to analyze an asymmetric kinetic resolution (KR), Scheme 3 (Wolf, C. (Ed.) “Dynamic Stereochemistry of Chiral Compounds,” RSC Publishing, Cambridge 29-135 (2008); Paivio et al., “Solvent-Free 15 Kinetic Resolution of Primary Amines Catalyzed by Candida Antarctica Lipase B: Effect of Immobilization and Recycling Stability,” Tetrahedron Asymm. 23:230-236 (2012), which are hereby incorporated by reference in their entirety). The outcome of the enzymatic KR of racemic amine 12 via amide formation with ethyl methoxyacetate catalyzed by Candida antarctica lipase B, Cal-B, was determined and compared with traditional NMR and chiral HPLC methods. The CD sensing analysis conducted directly without any work-up by using a small aliquot from the reaction mixture gave 28.5% of (S)-12 in 100.6:0.0 er. These results compared well with 28.7% based on NMR spectroscopy and >99.5:0.5 er determined by chiral HPLC after derivatization of 12 with benzoyl chloride and isolation of the corresponding N-(1-phenylethyl)benzamide prior to resolution on a Chiralcel OD column. The chiroptical reaction analysis generated accurate results at a much faster pace and with significantly less effort and waste production than the traditional methods. Moreover, the entire analytical task was considerably more streamlined and user-friendly than currently used protocols because it was accomplished with a single method (CD sensing) which replaced the customary dependence on two different techniques (NMR spectroscopy and chiral HPLC). It is belived that the sensing with sulfonyl chlorides and the new quantitative concentration and er CD sensing strategy described herein will become broadly useful in asymmetric reaction and biological sample analysis.

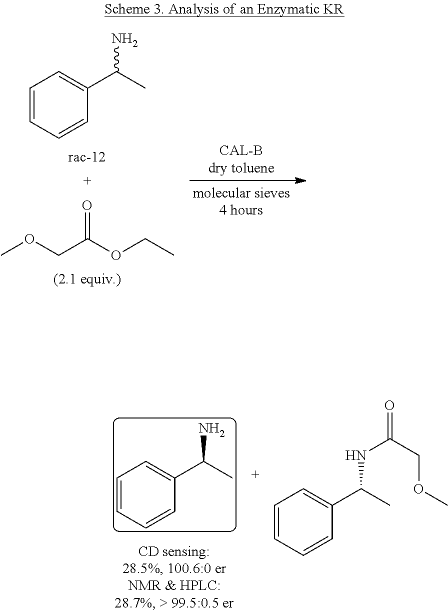

In conclusion, the present application describes a readily available achiral arylsulfonyl probes that allow quantitative chirality sensing of more than fifty amines, amino alcohols, and all standard chiral amino acids. In addition, a strategy that allows simultaneous concentration and er analysis based on the exclusive use of circular dichroism measurements is described. The chiroptical sensing is based on fast sulfonamide bond formation with stoichiometric probe amounts and can be performed in typical organic solvents or in aqueous solution by a simple mix-and-measure protocol and without the need to exclude air and moisture or other precautions. 2-Nitrobenzenesulfonyl chloride gave strong Cotton effects at long wavelengths with both aliphatic and aromatic substrates. The outstanding chiroptical properties, the general usefulness demonstrated with a very large group of chiral analytes, and the operational simplicity of chiral compound sensing with this sensor are highly advantageous features and pose an attractive alternative to traditional NMR spectroscopy or chiral chromatography methods. With this practical chirality probe a unified CD sensing method was developed that enables simultaneous concentration and er analysis which has not been possible to date. The experimental setup and the data processing are very practical and straightforward which is exemplified with the accurate determination of the absolute configuration, enantiomeric ratio, and concentration of 20 randomly prepared chiral amine and alanine samples, and with the streamlined analysis of the reaction outcome of an enzymatic kinetic resolution. This conceptually new approach toward determination of both concentration and er values solely by CD sensing offers significant speed, labor, and cost advantages at reduced chemical waste production compared to traditional techniques. Unified CD sensing is, of course, not limited to the use of arylsulfonyl chloride probes and expected to become broadly useful and can be easily adapted by many laboratories.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. An analytical method comprising:
providing a sample potentially containing a chiral analyte that can exist in stereoisomeric forms;
contacting the sample with a chromophore probe, wherein said contacting is carried out under conditions to permit binding of the chromophore probe to the chiral analyte, if present in the sample, to form a probe-labeled analyte; and
detecting the probe-labeled analyte in the sample using a single chiroptical assay format, and determining the concentration of the analyte in the sample and one or both of (i) the absolute configuration of the analyte in the sample, and (ii) the enantiomeric and/or the diastereomeric composition of the analyte in the sample.

2. The analytical method according to claim 1, wherein the chromophore probe covalently binds the chiral analyte to form the probe-labeled analyte.

3. The analytical method according to claim 1, wherein the chromophore probe non-covalently binds the chiral analyte to form the probe-labeled analyte.

4. The analytical method according to claim 1, wherein the chromophore probe is a metal salt, a quinone, a (hetero)aryl isocyanate, a (hetero)aryl isothiocyanate, a phenyl-naphthalene compound, an aryl halophosphite, an aryl halodiazaphosphite, a coumarin-derived Michael acceptor, a dinitrofluoroarene, an arylchlorophosphine, a metal complexed ligand, and a (hetero)arenesulfonyl compound.

5. The analytical method according to claim 4, wherein the chromophore probe is an achiral (hetero)arenesulfonyl compound having the structure according to formula (I):
Ar—SO₂—Z (I),
wherein Ar is a substituted or unsubstituted aromatic or heteroaromatic chromophore, and Z is a leaving group.

6. (canceled)

7. The analytical method according to claim 5, wherein the probe is an achiral (hetero)arenesulfonyl compound of Formula Ia: 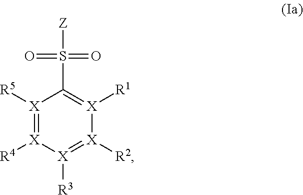
wherein:
each X is independently C or N, except that no more than three ring nitrogens are present in the (hetero)arenesulfonyl compound, and
R¹, R², R³, R⁴, and R⁵ are independently selected from the group consisting of a lone pair (when X is N), —H, —CN, —NO₂, halogen, —C₁-C₆ alkyl, —C₁-C₆ alkoxy, —N-(alkyl)₂, —C₁-C₆ alkenyl, —C₁-C₆ alkynyl, —C₁-C₆ perfluoroalkyl, -aryl, -perfluoroaryl, -aryloxy, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, —N-(heterocycloalkyl)₂, —C(O)Rₐ, —SO₂Rₐ, and —OC(O)Rₐ;
each Rₐ is independently selected from the group consisting of -alkyl, —O-alkyl, —N-(alkyl)₂, -alkenyl, -alkynyl, -aryl, —O-aryl, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, and —N-(heterocycloalkyl)₂; and
wherein, optionally, R¹ and R², R² and R³, R³ and R⁴, and/or R⁴ and R⁵ are alternatively taken together with the carbon or nitrogen atoms to which they are attached to form a fused monocyclic or bicyclic ring system selected from the group consisting of cycloalkyl, heterocycloalkyl, aryl, and heteroaryl, wherein the fused ring system is optionally substituted with one or more groups selected from -alkyl, —O-alkyl, —N-(alkyl)₂, -alkenyl, -alkynyl, —O-aryl, —O-heteroaryl, —N-(aryl)₂, —N-(heteroaryl)₂, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

8. (canceled)

9. The analytical method according to claim 5, wherein the probe is an achiral (hetero)arenesulfonyl compound according to Formula Ib: 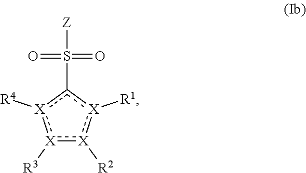
wherein:
each X is independently C or N; and
R¹, R², R³, and R⁴ are independently selected from the group consisting of —NCO, —NCS, a lone pair (when X is N), —H, —CN, —NO₂, halogen, —C₁-C₆ alkyl, —C₁-C₆ alkoxy, —N-(alkyl)₂, —C₁-C₆ alkenyl, —C₁-C₆ alkynyl, —C₁-C₆ perfluoroalkyl, -aryl, -perfluoroaryl, -aryloxy, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, —N-(heterocycloalkyl)₂, —OH, —C(O)Rₐ, —SO₂Rₐ, and —OC(O)Rₐ;
wherein each Rₐ is independently selected from the group consisting of —H, -alkyl, —O— alkyl, —N-(alkyl)₂, -alkenyl, -alkynyl, -aryl, —O-aryl, —N-(aryl)₂, -heteroaryl, —O-heteroaryl, —N-(heteroaryl)₂, -cycloalkyl, —O-cycloalkyl, —N-(cycloalkyl)₂, -heterocycloalkyl, —O-heterocycloalkyl, and —N-(heterocycloalkyl)₂; and
wherein, optionally, R¹ and R², R² and R³, and/or R³ and R⁴ is alternatively taken together with the carbon atoms to which they are attached to form a fused monocyclic or bicyclic ring system selected from the group consisting of cycloalkyl, heterocycloalkyl, aryl, and heteroaryl, wherein the fused ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

10. (canceled)

11. The analytical method according to claim 5, wherein the probe is selected from:

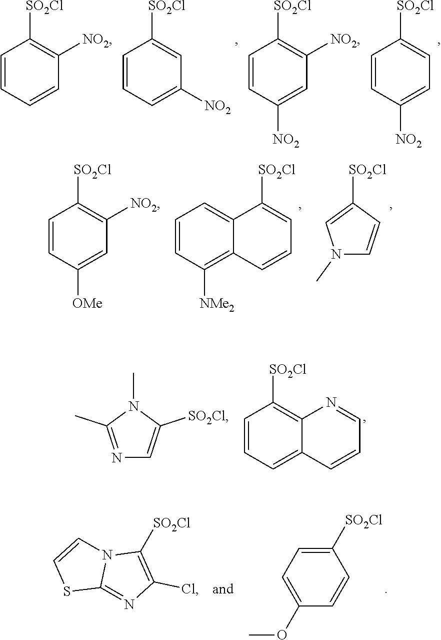

12. The analytical method according to claim 1, wherein the chiroptical assay format is circular dichroism (CD), vibrational CD (VCD), electronic CD, optical rotatory dispersion (ORD), or polarimetry.

13. The analytical method according to claim 1, wherein the chiral analyte and the corresponding probe are selected from the group consisting of:

| Probe | Chiral Analyte |
|---|---|
| Metal salts | amino acids, a-hydroxy acids, amino |
| | phosphonic acids, amino alcohols, and amines; |
| Substituted phenyl- | monoamines, amino alcohols, amino acids, and |
| naphthalene | combinations thereof; |
| aryl halophosphite | primary amines, secondary amines, amino |
| | alcohols, alcohols, hydroxy acids, amino acids, |
| | and combinations thereof; |
| halodiazaphosphite | primary amines, secondary amines, amino |
| | alcohols, alcohols, hydroxy acids, amino acids, |
| | and combinations thereof; |
| coumarin-derived | primary amines, secondary amines, amino |
| Michael acceptor | alcohols, alcohols, hydroxy acids, amino acids, |
| | and combinations thereof; |
| dinitrofluoroarene | primary amines, secondary amines, amino |
| | alcohols, alcohols, hydroxy acids, amino acids, |
| | and combinations thereof; |
| arylchlorophosphine | primary amines, secondary amines, amino |
| | alcohols, alcohols, hydroxy acids, amino acids, |
| | and combinations thereof; |
| an achiral quinone | amines, amino acids, amino alcohols, and |
| | combinations thereof; |
| (hetero)aryl | amines, amino acids, amino alcohols, diols, |
| isocyanate | alcohols, and combinations thereof; |
| (hetero)aryl | amines, amino acids, amino alcohols, diols, |
| isothiocyanate | alcohols, and combinations thereof; |
| (hetero)arenesulfonyl | primary amines, secondary amines, amino |
| compound | alcohols, amino acids, and combinations thereof; |
| Metal complexed | amines, diamines, a-hydroxy acids, amino acids, |
| ligands | and amino alcohols. |

14. (canceled)

15. The analytical method according to claim 1, wherein said contacting is carried out in a solvent selected from chloroform, dichloromethane, acetonitrile, toluene, tetrahydrofuran, methanol, ethanol, isopropanol, water, dimethyl sulfoxide (DMSO), dimethylformamide (DMF), pentane, pentane isomers, hexane, hexane isomers, ether, dichloroethane, acetone, ethyl acetate, butanone, and mixtures of any combination thereof.

16. The analytical method according to claim 1, wherein said contacting is carried out in an aqueous environment.

17. The analytical method according to claim 1, wherein said contacting is carried out in the presence of a base.

18. The analytical method according to claim 1, wherein said contacting is carried out in the presence of a buffer.

19. The analytical method according to claim 1, wherein said contacting is carried out for about 1 to about 300 minutes.

20.-23. (canceled)

24. The analytical method according to claim 1, wherein the absolute configuration of the analyte in the sample is determined.

25. The analytical method according to claim 1, wherein the enantiomeric and/or diastereomeric composition of the analyte in the sample is determined.

26. (canceled)

27. The analytical method according to claim 1, wherein
said contacting the sample is carried out on at least three measurements to which different known concentrations of the chromophore probe are introduced, wherein one of the at least three measurements comprises an excess concentration of the chromophore probe; and
said determining comprises plotting recorded intensity measurements from the chiroptical assay (y-axis) versus the chromophore probe concentration (X-axis) for the at least three measurements, and analyzing the plotted data using linear regression analysis.

28.-30. (canceled)

31. A kit comprising:
an aqueous or non-aqueous solution comprising a chromophore probe; and
optionally one or more of
(i) sample tubes suitable for use with a spectrophotometer;
(ii) an optically pure reference sample of an analyte,
(iii) directions for using a spectrophotometer for carrying out circular dichroism (CD), vibrational CD (VCD), electronic CD, optical rotatory dispersion (ORD), or polarimetry analyses to measure the concentration of an analyte in a sample and one or both of the absolute configuration of the analyte in the sample, and the enantiomeric and/or the diastereomeric composition of the analyte in the sample, and
(iv) a recordable medium comprising a template for analyzing data obtained from the spectrophotometer and determining the concentration of an analyte in a sample and one or both of the absolute configuration of the analyte in the sample, and the enantiomeric and/or the diastereomeric composition of the analyte in the sample.

32-33. (canceled)
